(19) United States
(12) Patent Application Publication
(10) Pub. No.: US 2024/0400627 A1
(43) Pub. Date: Dec. 5, 2024

(54) COMPOSITIONS AND METHODS FOR THE TREATMENT OF AUTOSOMAL DOMINANT POLYCYSTIC KIDNEY DISEASE AND OTHER DISEASES HAVING UPREGULATED MTOR ACTIVITY

(71) Applicant: Yale University, New Haven, CT (US)

(72) Inventors: Michael J. Caplan, Woodbridge, CT (US); Laura Onuchic, New Haven, CT (US); Valeria Padovano, Medford, MA (US); Carson Thoreen, Guilford, CT (US)

(21) Appl. No.: 18/697,709

(22) PCT Filed: Sep. 30, 2022
(86) PCT No.: PCT/US2022/077393

§ 371 Date: Apr. 1, 2024

Related U.S. Application Data
(60) Provisional application No. 63/250,663, filed on Sep. 30, 2021.

(51) Int. Cl. C07K 14/47; A61K 38/00; A61K 48/00; A61P 13/12

(52) U.S. Cl. CPC C07K 14/4702; A61K 48/0008; A61K 48/0058; A61P 13/12; A61K 38/00; C07K 2319/07

(57) ABSTRACT

PC1-CTT polypeptides for the treatment of Autosomal Dominant Polycystic Kidney Disease (ADPKD) are provided and can be or include PC1-CTT (SEQ ID NO:1) or a functional fragment or variant thereof. In some embodiments, the PC1-CTT polypeptide is a fusion protein or conjugate further including a functional element such as a protein transduction domain, fusogenic polypeptide, targeting signal, or expression and/or purification tag. Nucleic acids encoding the disclosed PC1-CTT polypeptides and other therapeutic proteins are also provided. In some embodiments, the nucleic acid encodes a TOP or TOP-like motif. The nucleic acids can be RNA or DNA, and can be, for example, a vector such as a plasmid or viral vector, or an mRNA. Methods of treatment are provided and typically include administering a subject in need thereof an effective amount of a disclosed polypeptide or nucleic acid.

### CROSS-REFERENCE TO RELATED APPLICATION

This application claims the benefit of and priority to U.S. Ser. No. 63/250,663 filed Sep. 30, 2021, and which is incorporated by reference in its entirety.

### STATEMENT REGARDING FEDERALLY SPONSORED RESEARCH

This invention was made with government support under DK120534 and CA233096 awarded by National Institutes of Health. The government has certain rights in the invention.

### REFERENCE TO SEQUENCE LISTING

The Sequence Listing submitted as an .xml file named “YU_8214_PCT_ST26.xml,” created on Sep. 30, 2022, and having a size of 77,082 bytes, is hereby incorporated by reference pursuant to 37 C.F.R § 1.834(c)(1).

### FIELD OF THE INVENTION

The invention is generally directed to compositions and method of treating Autosomal Dominant Polycystic Kidney Disease and other diseases characterized by dysregulated mTOR activity.

### BACKGROUND OF THE INVENTION

Autosomal Dominant Polycystic Kidney Disease (ADPKD) is the most common potentially lethal genetic disease and the most common genetic cause of end stage renal failure. ADPKD affects ˜1:1,000 people and individuals with ADPKD account for ˜10% of patients requiring renal replacement therapy. ADPKD is characterized by the progressive development of fluid-filled cysts whose expansion compromise renal function and can lead to end-stage renal disease. Although PKD1 was identified over 25 years ago (Consortium, Cell Vol. 78, 725 (1994)), current therapeutic options remain limited and incompletely effective. Metabolic abnormalities, including a “Warburg-like” effect marked by increased glycolysis and lactate production, defective fatty acid oxidation, and decreased rates of oxidative phosphorylation, have emerged as hallmarks of ADPKD (Padovano, Nat Rev Nephrol Vol. 14, 678-687 (2018), Priolo & Henske, Nat Med Vol. 19, 407-9 (2013), Rowe, et al., Nat Med Vol. 19, 488-93 (2013), Chiaravalli, al., J Am Soc Nephrol Vol. 27, 1958-69 (2016), Padovano, et al., Mol Biol Cell Vol. 28, 261-269 (2017)). ADPKD is also characterized by large fluid-filled renal cysts that remodel, compress and destroy surrounding normal tissue, and that progressively reduce kidney function, leading to end stage renal disease in about 50% of patients by the sixth decade of life. Most ADPKD results from mutations in two genes, PKD1, which encodes the polycystin-1 protein (PC1), and PKD2, which encodes the polycystin-2 protein (PC2). PC1 and PC2 interact with one another and are thought to play a role in cilia signaling. It is generally accepted that the cilium is a central component in the pathways that drive ADPKD pathogenesis. Although their mechanistic connection to the functional PC complex in cilia is unclear, numerous signaling pathways are perturbed in cysts.

There are currently very limited options for ADPKD therapy. A limited number of drugs licensed by the U.S. Food and Drug Administration (U.S. FDA) for treatment of PKDs include tolvaptan and lixivaptan, which are non-peptide, small molecule agents that act as competitive vasopressin receptor type 2 antagonists. Tolvaptan and lixivaptan slow cyst growth by 50% compared to placebo while decreasing progression of kidney failure by approximately 30% in large clinical trials (Tones, et al., N Engl J Med 367(25):2407 (2012). The FDA has restricted use of tolvaptan to no more than 30 days continuously due to concerns about severe liver injury. Given the ongoing nature of PKD, this makes it highly unlikely these drugs would be safe. Additionally, at best, they only slow progression of PKD.

Many other treatments have been suggested as potential therapies, but all have failed so far. Perhaps the most spectacular were the mammalian target of rapamycin (mTOR) inhibitor drugs sirolimus and everolimus. Many lines of evidence indicated that mTOR promotes cyst growth in people with PKDs, and studies in animal models were promising. However, human clinical trials of both agents found no clinical benefit and even some evidence of harm (Serra, et al., N Engl J Med 363(9):820-9 (2010); Walz G, et al., N Engl J Med 363(9):830-40 (2010).

Drinking large amounts of water has also been posited as a way to delay cyst progression, and it seemed to have this effect in rodent models of PKD (Nagao S, et al., J Am Soc Nephrol, 17(8):2220-7 (2006). Unfortunately, the one human trial of this approach found the opposite, with signs of increased urine protein loss in patients assigned to drink large amounts of water compared to those drinking usual amounts (Higashihara, et al., Nephrol Dial Transplant 29(9):1710-9 (2014)).

Pravastatin is a well-known drug used primarily for lowering cholesterol levels to reduce cardiovascular disease risks. A single clinical trial in children with ADPKD showed that it was able to modestly slow progression of cyst growth (Cadnapaphornchai, et al., Clin J Am Soc Nephrol 9(5):889-96 (2014)).

The drug metformin, known as an insulin sensitizing agent, activates 5′-adenosine monophosphate-activated protein kinase (AMPK). Its use has been associated with reduction in cyst formation in animal models of ADPKD (Chang, et al., Sci Rep 7(1):7161 (2017)). The natural product berberine, which is believed to act by a similar mechanism as metformin, has shown similar efficacy in preclinical research (Bonon, et al., Biochem Biophys Res Commun 441(3):668-74 (2013)).

In summary, roughly 50% of ADPKD patients will require renal replacement therapy, which is both extremely expensive and associated with high levels of morbidity and mortality. The only approved pharmacotherapy has limited efficacy, is extremely expensive, and needs to be administered on a daily basis for decades. There is no known treatment that actually reverses any form of PKD. A handful of agents may mildly slow progression, and to date it is believed that no strategies for gene therapy of ADPKD have been developed.

Thus, it is an object of the invention to provide compositions and methods of use thereof for the treatment of ADPKD, and other diseases characterized by dysregulated mTOR.

### SUMMARY OF THE INVENTION

The results presented in the experiments below show that transgenic expression of a 200 amino acid fragment of the Polycystin-1 protein in a mouse model of ADPKD is sufficient to suppress disease development, and its activity is likely tied to a domain that is important for interaction with nicotinamide nucleotide transhydrogenase (NNT). Thus, compositions including therapeutic polypeptides and nucleic acids encoding the same, and methods of use thereof for the treatment of ADPKD are provided.

Kidney cells affected by ADPKD exhibit very high levels of activity of mTOR signaling pathway. Thus, also provided is a strategy for expression of a therapeutic polypeptide of interest in cells that manifest high mTOR activity, by incorporating an mTOR translation-responsive nucleic acid sequence into a nucleic acid encoding the therapeutic protein. In some embodiments, the therapeutic polypeptide is a C-terminal fragment of Polycystin-1 or a variant thereof, optionally, but preferably for the treatment of ADPKD.

Many other diseases in addition to ADPKD also exhibit high levels of mTOR. Thus, this strategy can also be used in other gene therapy compositions and methods. Therefore, in some embodiments, the therapeutic polypeptide is not a fragment of Polycystin-1, but rather a different therapeutic polypeptide, and is for use in the treatment of a different disease.

Compositions and methods disclosed herein, particularly gene therapy compositions, have the potential to be curative with a single administration, and thus may reduce the expenses and work associated with the care of patients with ADPKD, and possible other disorders.

Thus disclosed are compositions including polypeptides and nucleic acids encoding the same, each of which can be packaged in delivery vehicles, and any of which can include additional moieties to enhance delivery in cells, optionally specific cells, and bioactivity therein.

For example, in some embodiments, PC1-CTT polypeptides are provided and can be or include PC1-CTT (SEQ ID NO:1) or a functional fragment or variant thereof. In some embodiments, the PC1-CTT polypeptide is a fusion protein or conjugate further including a functional element such as a protein transduction domain, fusogenic polypeptide, targeting signal, or expression and/or purification tag. Thus, the PC1-CTT polypeptide can be a fusion protein including a PC1-CTT sequence or fragment or variant thereof, and a heterologous sequence. In some embodiments, the variant includes at least 70% sequence identity of SEQ ID NO:1, or a functional fragment thereof. The variant or fragment can be between 25 and 200 amino acids inclusive, or any subrange or specific integer therebetween. Typically, the polypeptide is not full-length Polycystin-1, and is optionally, but preferably less than 1,000, more preferably less than 500 or less than 250 amino acids. In preferred embodiments, the polypeptide can interact with nicotinamide nucleotide transhydrogenase (NNT), optionally wherein the interaction includes the ability to co-immunoprecipitate. In some embodiments, the polypeptide includes a mutated PEST motif with reduced or eliminated activity. Typically, the PC1-CTT polypeptide includes a mitochondrial localization sequence (MLS). The MLS can be an endogenous MLS (e.g., SEQ ID NOS:98 or 99), or a variant thereof, e.g., with at least 70% sequence identity thereto. Additionally or alternatively, the MLS can be a heterologous MLS that substituted for endogenous MLS, and/or otherwise inserted or appended to the polypeptide, e.g., at the N- or C-terminus.

Nucleic acids encoding the disclosed PC1-CTT polypeptides and other therapeutic proteins are also provided. In some embodiments, the nucleic acid encodes a TOP or TOP-like motif. Typically, the TOP or TOP-like motif includes at least 4 pyrimidines beginning within four nucleotides of the transcriptional start site. In some embodiments, the TOP motif is, or includes, a C at the +1 position of the mRNA followed by at least 4 pyrimidines. Exemplary sequences including or consisting of TOP and TOP-like motifs include the nucleic acid sequences of the underlined portion of any of SEQ ID NOS:21-52 of Table 1, and/or any of SEQ ID NOS:21-52 or 87.

The nucleic acids can be, for example, RNA or DNA, and optionally include an expression control sequence(s) such as promoter, which may be cell type specific (e.g., kidney epithelial cells). In some embodiments, the nucleic acid is a vector such as a plasmid or viral vector, or an mRNA. In some embodiments, the nucleic acid has one or more functional elements such as a protein transduction domain, fusogenic polypeptide, and/or targeting signal conjugated thereto.

Any of the disclosed polypeptides and nucleic acids can be package in or otherwise associated with a delivery vehicle. Exemplary delivery vehicles are also provided and can be, for example, polymeric particles, inorganic particles, silica particles, liposomes, micelles, or multilamellar vesicles. Optionally, the delivery vehicles have one or more of a protein transduction domain, fusogenic polypeptide, and/or targeting signal conjugated thereto.

Pharmaceutical compositions are also provided and can include a pharmaceutically acceptable carrier and any of the disclosed polypeptides or nucleic acids alone, or packaged in a delivery vehicle.

Methods of treatment are provided and typically include administering a subject in need thereof an effective amount of a disclosed polypeptide or nucleic acid, optionally, but preferably, in a pharmaceutical composition. In particular embodiments, the subject has a genetic disorder such as Autosomal Dominant Polycystic Kidney Disease (ADPKD), and the composition reduces or prevents one or more symptoms thereof. Methods of administration are also provided and include, but are not limited to, parenteral routes such as intravenous injection or infusion. In some methods, particularly where the kidneys are the target tissue, the compositions are administered by a retrograde ureteral approach.

Methods of treating a subject with a disease characterized by increased mTOR activity are also provided and typically include administering a subject a nucleic acid including a sequence encoding a therapeutic polypeptide and a TOP or TOP-like motif. The TOP and TOP-like motifs are used to drive preferential expression of a therapeutic peptide in affected cells that manifest elevated levels of mTOR activity. Exemplary diseases include, but are not limited to, ADPKD, arthritis, insulin resistance, osteoporosis, cancer, and mTOR-opathies such as tuberous sclerosis complex (TSC), focal cortical dysplasia type II (FCDII), hemimegaloencephaly (HME), polyhydramnios, megalocephaly, or symptomatic epilepsy (PMSE) syndrome. When the disease is a genetic disorder, the therapeutic polypeptide can be, for example, a wildtype copy or other fragment or variant thereof that restores the function or bioactivity lost by the mutated gene/protein of the genetic disorder. When the disease is cancer, the therapeutic polypeptide can be one that induces cancer cell death.

### BRIEF DESCRIPTION OF THE DRAWINGS

FIG. 1 is a graphic representation of PC1 N-terminal cleavage, C-terminal cleavage, and generation of the PC1 C-terminal tail (PC1-CTT), that can enter the nucleus and mitochondria.

FIG. 2A is a schematic representation of the design of the 2HA-PC1-CTT; Pkd1^{fl/fl}; Pax8^{rtTA}; TetO-Cre (Pkd1-KO+CTT) mouse model. transgenic mice that carry a BAC-2HA-PC1-CTT transgene inserted in the Rosa26 locus and preceded by a neomycin resistance (NeoR) STOP cassette flanked by loxP sequences. These mice were crossed with the previously characterized Pkd1^{fl/fl}; Pax8^{rtTA};TetO-Cre mouse model of ADPKD in which exons 2-4 of the Pkd1 gene are flanked by loxP sequences (Ma, M., et al. Nature Genetics Vol. 45, pages 1004-12 (2013);⁴³. Cre-mediated recombination of these second-generation 2HA-PC1-CTT; Pkd1^{fl/fl};Pax8^{rtTA};TetO-Cre mice via doxycycline induction promotes 2HA-PC1-CTT expression and loss of full-length PC1 in tubular epithelial cells.

FIG. 2B-2D are bar graphs of comparative analysis of N-Pkd1-KO; N-Pkd1-KO+CTT and N-WT mice showing differences in KW/BW ratio (FIG. 2B), BUN (FIG. 2C) and serum creatinine (FIG. 2D). “N” mice refers to a C57BL6N, NNT-competent background. Cystic mouse cohorts are composed of 53%-58% female and 42%-47% male mice.

FIG. 3 is an image comparing gross mouse kidney anatomy from two induced female littermates, in which one animal did not inherit the BAC PCT1-CTT transgene (left) and the other inherited the transgene and exhibited a rescue in phenotype (right).

FIG. 4A is an experimental workflow for the identification of protein interaction partners from mitochondrial-associated pools of PC1 and its fragments. Crude mitochondria fractions prepared from Pkd1^{F/H}-BAC and WT mice were solubilized, crosslinked with 3 mM DTSSP, and subjected to immunoprecipitation with anti-HA antibodies. The proteins recovered were identified by mass spectrometry. HA-HRP immunoblotting of the crude mitochondrial HA-immunoprecipitate revealed recovery of both the CTF of the full-length 3FLAG-PC1-3HA and the PC1-CTT-3HA fragments only in immunoprecipitates from Pkd1^{F/H}-BAC mice. FIG. 4B is a volcano plot of PC1 and PC1-CTT interacting partners from Pkd1^{F/H}-BAC vs WT kidneys (Colored dots: P<0.05 determined by Fisher's exact test; n=3 per group). The amount of NNT detected as co-immunoprecipitating with CTT in Pkd1^{F/H}-BAC kidney immunoprecipitates were >16-fold greater than that detected in control WT immunoprecipitates (P<10-10). Other PC1-relevant hits specific to the endoplasmic reticulum (ER) proteome were identified, consistent with the fact that the crude mitochondria fractions contain MAMs. This is the case for the observed 4-fold enrichment of Ganab (P<0.05), which encodes the glucosidase II α-subunit. Of note, mutations in GANAB are a rare cause of ADPKD (Porath, et al., Am J Hum Genet, 98, 1193-1207 (2016), Besse, et al., J Clin Invest 127, 1772-1785 (2017)). FIG. 4C is a bar graph showing results from the Mander's colocalization analysis of the fraction of CTT associated with mitochondria which revealed an overlap coefficient of 0.8535. This value is indicative of extensive overlap between the distribution of CTT and NNT. The TOMM20/NNT colocalization coefficient was measured as an experimental positive control and, is slightly but significantly greater than the coefficient calculated for the CTT/NNT co-localization. Data are expressed as mean±SEM. Pairwise comparison was performed using Student's t-test.

FIGS. 5A-5C are dot plots showing repeats of the experiment in the Pax8^{rtTA}; TetO-Cre; Pkd1^{fl/fl} C57BL6J mouse model (“J” mice). “J” mice refer to a C57BL6J, NNT-deficient background. Comparative analysis between Pkd1-KO+CTT and Pkd1-KO in the “J” background revealed no significant change in KW/BW ratio (A), BUN (B), and serum creatinine levels (C). Cystic mouse cohorts are composed of 53%-55% female and 45%-47% male mice. Multiple group comparisons were performed using one-way ANOVA followed by Tukey's multiple-comparisons test. FIG. 5D is a dot plot illustrating quantification of tubular and cystic area in H&E-stained kidney sections from Pkd1-KO and Pkd1-KO+CTT mice on both “N” and “J” backgrounds, as determined by ImageJ using images of renal cross-sections. FIG. 5E is a dot plot of the quantification of tubular proliferation determined by the percentage of ki67 positive nuclei in renal tubular epithelial cells. Counting of ki67 positive nuclei was performed by an individual blinded to the experimental conditions. Data are expressed as mean±SEM. Pairwise comparisons were performed using Student's t-test for FIGS. 5D and 5E.

FIG. 6A is a schematic of an experimental timeline of the development of the 2HA-PC1-CTT; Pkd1^{fl/fl}; Pkhd1-Cre mouse model. The 2HA-PC1-CTT;Pkd1fl/fl;Pax8rtTA;TetO-Cre mouse model on the “N” background (N-Pkd1-KO+CTT) was crossed with the developmental Pkd1^{fl/fl};Pkhd1-Cre (Pkhd1-Cre;Pkd1-KO) (Ma, M., et al. Nature Genetics Vol. 45, pages 1004-12 (2013); Fedeles, S. V. et al. Nature Genetics Vol. 43, pages 639-47 (2011)).^{20,34} ADPKD mouse model, generated on the “J” background. Breeders with a Pkd1^{fl/+};Pkhd1-Cre genotype were used, due to the constitutive activity of the Pkhd1-Cre that initiates collecting duct specific cre-mediated recombination during embryonic stages, and prevents animals with the complete Pkd1^{fl/fl};Pkhd1-Cre from reaching sexual maturity. The F1 progeny were heterozygous for Nnt and Pkd1^{fl/fl};Pkhd1-Cre (Pkhd1-Cre;Pkd1-KO).+/−CTT littermates were evaluated at p14 (post-partum day 14). FIG. 6B is a dot plot showing results from comparative analysis of Pkhd1-Cre;Pkd1-KO, Pkhd1-Cre;Pkd1-KO+CTT, WT, and Pkhd1-Cre+CTT mice, showing differences in KW/BW ratio. Of note, no phenotypic differences were observed in WT CTT-expressing mice (Pkhd1-Cre+CTT).

FIGS. 7A and 7B are Principal Component Analysis (PCA) plots of Liquid-Chromatography Mass-Spectrometry (LC-MS) based metabolomic data from Pkd1-KO+CTT mice vs Pkd1-KO mice which revealed clear separation between groups in the “N” background but not in the “J” background. Each individual mark corresponds to a different sample and its location in the plot is determined by the relative contributions of subsets of metabolites to the variance among samples. FIGS. 7C and 7D are volcano plots showing differences in metabolic profiling of the kidney extracts from Pkd1-KO+CTT mice vs Pkd1-KO mice in the “N” and “J” backgrounds. The vertical lines in each panel mark 2-fold changes; horizontal lines mark P<0.05 determined by Student's t-test; n=5 mice (N-Pkd1-KO) and n=6 mice for N-Pkd1-KO+CTT, J-Pkd1-KO and J-Pkd1-KO+CTT groups. Colored dots indicate metabolites with significant fold changes. Forty-four metabolites met both criteria of P-value<0.05 and fold change>2, 6 of them upregulated and 38 downregulated in N-Pkd1-KO+CTT compared to N-Pkd1-KO kidneys. In contrast, analysis of both J-Pkd1-KO+CTT and J-Pkd1-KO revealed a significant change in only 1 metabolite. The identification and labeling of each metabolite identified in FIG. 7C is shown The set of downregulated metabolites in N-Pkd1-KO+CTT mice included several metabolites relevant to ADPKD, such as methionine (Padovano, et al., Nat Rev Nephrol 14, 678-687 (2018)), lactate (Priolo & Henske, Nat Med 19, 407-9 (2013), Rowe, et al., Nat Med 19, 488-93 (2013)), asparagine (Chiaravalli, et al., Soc Nephrol 27, 1958-69 (2016), Padovano, et al., Mol Biol Cell 28, 261-269 (2017)) and glutamate (Chiaravalli, et al., Soc Nephrol 27, 1958-69 (2016)), as well as uremic toxins (allantoin and 5-hydroxyindoleacetate) and urea cycle metabolites. Complete untargeted comparative metabolomic analysis is provided in FIG. 7E is a heat map showing altered metabolites in bulk kidney tissue. FIGS. 7F-7J are dot plots showing quantification of immunoblot data from total kidney lysate from N-Pkd1-KO+CTT and N-Pkd1-KO mice using a “mitococktail” antibody that reports on the assembly status of mitochondrial complexes I, II, III, IV and V. Blots were also probed with antibodies directed against TOMM20 and NNT. Blotting for actin served as a loading control. A relative increase in assembly of CIV (FIG. 7J) and CV (FIG. 71) was observed in N-Pkd1-KO+CTT vs N-Pkd1-KO. No differences were observed in the assembly of complexes I (CI), II (CII) or III (CIII) in these same mice. The same comparison on the “J” background reveals no influence of CTT expression on mitochondrial mass or on mitochondrial complex assembly. Data are expressed as mean±SEM. Pairwise comparisons were performed using Student's t-test. Additional comparative assessment of mitochondrial mass and mitochondrial complex assembly were conducted.

FIGS. 8A and 8B are dot plots showing gender dimorphism is significantly more pronounced in the J background when compared to the N background, as shown by Kidney/Bodyweight (6A) and BUN (6B) measurements. FIG. 8C is dot plot showing creatinine levels are elevated in ADPKD mice on the “N” background when compared to “J” background. Data are shown as mean+/−SEM; p values determined by t test; male (darker dots) and female (lighter dots) mice as indicated in grayscale. FIG. 8D is a series of images comparing kidney morphology in “N” and “J” PC1 KO mice with and without PC1-CTT expression.

FIG. 9A is a schematic representation depicting NNT and its localization to the inner mitochondrial membrane and mitochondrial matrix, as well as its dependence upon the mitochondrial proton gradient to catalyze the transfer of a hydride between NADH and NADP+ (forward enzymatic activity). FIGS. 9B and 9C are dot plots illustrating LC-MS detection of NAD(P)(H) cofactors showing significant differences in NADPH/NADP+ (FIG. 9B) and NADH/NAD+ (FIG. 9C) ratios between Pkd1-KO+CTT and Pkd1-KO mice exclusively in the “N” background. FIG. 9D is a schematic representation of the experimental timeline for assessment of NNT enzymatic activity in pre-cystic mice at 10 weeks of age. FIGS. 9E and 9F are dot plots showing kidney function is preserved in all three 10-week mouse cohorts (N-Pkd1-KO, N-Pkd1-KO+CTT and N-WT) as revealed by normal values of BUN (FIG. 9E) and serum creatinine (FIG. 9F) across the groups. Cystic mouse cohorts are composed of 45%-50% female and 50%-55% male mice. FIG. 9G is a line graph of NNT activity in N-Pkd1-KO, N-Pkd1-KO+CTT and N-WT mice, quantified using a kinetic spectrophotometric assay that measures reduction of the NAD analog APAD. The measurements were made over the course of 180 s and samples were normalized to protein content. WT “J” mice served as experimental negative controls and confirmed the specificity of the assay by showing the absence of an upward slope. FIG. 9H is a dot plot illustrating quantification of Immunoblot of mitochondrial extract from N-Pkd1-KO, N-Pkd1-KO+CTT and N-WT mouse kidneys. NNT expression (normalized to VDAC) was not significantly different across all groups of 10-week-old mice. FIG. 9I is a dot plot of the comparison of NNT activity among N-Pkd1-KO, N-Pkd1-KO+CTT and N-WT mice, measured as AOD variation/s/mg of protein. Data are expressed as mean±SEM. Pairwise comparisons were performed using Mann-Whitney U test due to non-normally distributed data (FIGS. 9B and 9C). Multiple group comparisons were performed using one-way ANOVA followed by Tukey's multiple-comparisons test (FIGS. 9E, 9F, 9H and 9I). FIGS. 10A-10C are dot plots illustrating comparative assessment of renal phenotype between N-Pkd1-KO and J-Pkd1-KO mice, including KW/BW ratio (FIG. 10A), BUN (FIG. 10B), and serum creatinine (FIG. 10C). Both cystic mouse cohorts were composed of 53% female and 47% male mice.

FIGS. 11A and 11B are graphs illustrating generation of a TERT immortalized Pkd−/− cell line and its use to compare the activity of PC1-CTT, EV, PC1-CTTΔMTS, and EV(-NADPH). Results show that the NNT activity in this cell line, which lacks polycystin-1 expression, can be substantially increased by expressing the polycystin-1 C-terminal tail (PC1-CTT), and this effect does not occur if the polycystin-1 C-terminal tail lacks the mitochondrial localization sequence (PC1-CTTΔMTS).

FIGS. 12A and 12B show results from immunoblots of 60 μg of total kidney lysate from WT, N-Pkd1-KO+CTT and BAC-Pkd1 mice. Actin served as loading control. The 150-kDa bands exclusive to BAC-Pkd1 mice represent the PC1-CTF fragment that results from N-terminal cleavage of full-length PC1 at the GPS site (Padovano, et al., Cell Signal 72, 109634 (2020)). Lysates from Pkd1^{F/H}-BAC mice showed the same 37-kDa C-terminal HA-tagged tail fragment band as the CTT-expressing Pkd1-KO mice (FIG. 12A), which is detected at similar levels (FIG. 12B), indicating an upper threshold for CTT expression in the N-Pkd1-KO+CTT mice of approximately 1.5-fold above the levels expected for WT mice. Data are expressed as mean±SEM. Pairwise comparisons were performed using Student's t-test.

FIGS. 13A-13D are bar graphs showing that Pkd1-KO+CTT and Pkd1-KO on the “N” (FIGS. 13A and 13B) and “J” (FIGS. 13C and 13D) backgrounds demonstrate random distribution of homozygosity or heterozygosity status for both Pax8^{rtTA} and TetO-Cre alleles. These parameters do not correlate with phenotype severity in any of the four groups, as determined by the KW/BW ratio. FIG. 13E is a schematic representation of qPCR primers capable of exclusively detecting genomic DNA sequence encoding full-length endogenous PC1 from cells that did not undergo Cre-recombination in Pkd1-KO mice. The reverse primer is specific for Pkd1 exon 4 and the forward primer is specific to its preceding intron. Primer positions were based on the mouse genome assembly GRCm39. FIG. 13F is a bar graph of the comparative analysis of PC1 rearrangement levels across all mouse cohorts. Levels of non-rearranged WT Pkd1 was determined by extracting genomic DNA from kidney tissue from each mouse contained in the cohort followed by quantitative genomic PCR using primers described in FIG. 13E. The levels of non-rearranged WT Pkd1 were normalized to levels detected in WT controls. The fractional extent of rearrangement is unchanged across the four groups. Data are expressed as mean±SEM. Multiple group comparisons were performed using one-way ANOVA followed by Tukey's multiple-comparisons test.

FIG. 14 is a bar graph of the frequency of the Crb1^{rd8} mutant allele in cystic mice on the “N” background.

FIGS. 15A-15H are dot plots showing comparisons of normalized band intensities representative of both mitochondrial complex assembly and mitochondrial mass that did not differ between cystic mice that do or do not express CTT in both “N” (FIGS. 15A-15C) and “J” (FIGS. 15D-15H) backgrounds. Data are expressed as mean±SEM. Pairwise comparisons were performed using Student's t-test.

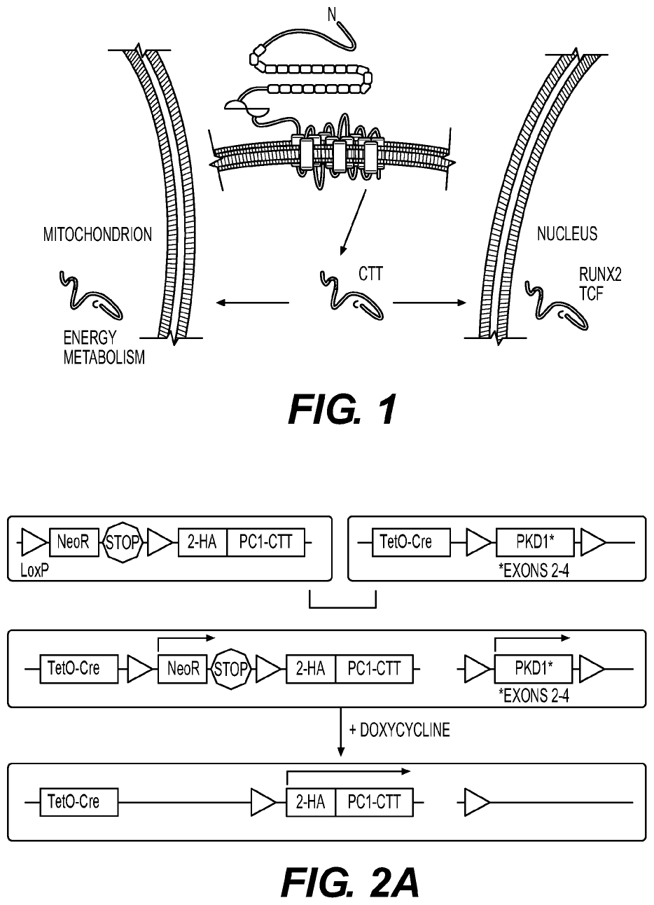

### DETAILED DESCRIPTION OF THE INVENTION

### I. Definitions

As used herein, the term “treat” means to prevent, reduce, decrease, or ameliorate one or more symptoms, characteristics or comorbidities of an age-related disease, disorder or condition; to reverse the progression of one or more symptoms, characteristics or comorbidities of an age related disorder; to halt the progression of one or more symptoms, characteristics or comorbidities of an age-related disorder; to prevent the occurrence of one or more symptoms, characteristics or comorbidities of an age-related disorder; to inhibit the rate of development of one or more symptoms, characteristics or comorbidities or combinations thereof.

As used herein, the terms “individual,” “subject,” and “patient” are used interchangeably herein, and refer to a mammal, including, but not limited to, rodents, simians, and humans.

As used herein, the terms “reduce”, “inhibit”, “alleviate” and “decrease” are used relative to a control. One of skill in the art would readily identify the appropriate control to use for each experiment. For example, a decreased response in a subject or cell treated with a compound is compared to a response in subject or cell that is not treated with the compound.

As used herein, the terms “increase”, “induce”, “activate” and “improve” are used relative to a control. One of skill in the art would readily identify the appropriate control to use for each experiment. For example, an increased response in a subject or cell treated with a compound is compared to a response in subject or cell that is not treated with the compound.

As used herein, the term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “functional fragment” as used herein is a fragment of a full-length protein retaining one or more function properties of the full-length protein.

The term “transgenic” refers to an organism and the progeny of such and organism that contains a nucleic acid molecule that has been artificially introduced into the organism.

As used herein, the term “variant” refers to a polypeptide or polynucleotide that differs from a reference polypeptide or polynucleotide but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will assist the linked protein to be localized at the specific organelle.

As used herein, the term “Localization Signal or Sequence or Domain” or “Targeting Signal or Sequence or Domain” are used interchangeably and refer to a signal that directs a molecule to a specific cell, tissue, organelle, intracellular region or cell state. The signal can be polynucleotide, polypeptide, or carbohydrate moiety or can be an organic or inorganic compound sufficient to direct an attached molecule to a desired location.

As used herein, the term “cell surface marker” refers to any molecule such as moiety, peptide, protein, carbohydrate, nucleic acid, antibody, antigen, and/or metabolite presented on the surface or in the vicinity of a cell sufficient to identify the cell as unique in either type or state.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Compositions

### A. PC1-CTT Polypeptides

Compositions including and encoding a polypeptide including a PC1-CTT sequence, referred to herein as “PC1-CTT polypeptides”, are provided. As discussed in more detail below, the PC1-CTT polypeptides include or consist of the sequence of the 200 amino acid proteolytic fragment of endogenous human PC1 (referred to as “PC1-CTT”), or include or consist of a functional fragment or variant thereof. The PC1-CTT polypeptides, nucleic acids encoding the same, and delivery vehicles thereof can optionally include one or more heterologous sequences. Thus, the PC1-CTT can be a fusion protein.

### 1. PC1-CTT Sequences

PKD1 and PKD2 are two genes that have been identified as responsible for the vast majority of autosomal polycystic kidney disease, a common inherited disease that causes progressive renal failure. PKD1 encodes polycystin-1 (PC1), a large glycoprotein that contains several extracellular motifs indicative of a role in cell-cell or cell-matrix interactions (Tsiokas, et al., Proc Natl Acad Sci USA, 94(13): 6965-6970 (1997). PC1 undergoes C-terminal and N-terminal cleavage (Padovano, et al., Cell Signal, 72: p. 109634 (2020)). C-terminal cleavage generates fragment (PC1-CTT), that translocate to the nucleus (Chauvet, et al., J Clin Invest, 114(10): p. 1433-43 (2004)) and to the mitochondria (Lin, et al., Sci Rep, 8(1): p. 2743 (2018)).

A consensus amino acid sequence of PC1 is

(SEQ ID NO: 2)
MPPAAPARLALALGLGLWLGALAGGPGRGCGPCEPPCLCGPAPGAACRVNCSGRGLRTLGPA
LRIPADATALDVSHNLLRALDVGLLANLSALAELDISNNKISTLEEGIFANLENLSEINLSG
NPFECDCGLAWLPRWAEEQQVRVVQPEAATCAGPGSLAGQPLLGIPLLDSGCGEEYVACLPD
NSSGTVAAVSFSAAHEGLLQPEACSAFCFSTGQGLAALSEQGWCLCGAAQPSSASFACLSLC
SGPPPPPAPTCRGPTLLQHVFPASPGATLVGPHGPLASGQLAAFHIAAPLPVTATRWDFGDG
SAEVDAAGPAASHRYVLPGRYHVTAVLALGAGSALLGTDVQVEAAPAALELVCPSSVQSDES
LDLSIQNRGGSGLEAAYSIVALGEEPARAVHPLCPSDTEIFPGNGHCYRLVVEKAAWLQAQE
QCQAWAGAALAMVDSPAVQRFLVSRVTRSLDVWIGFSTVQGVEVGPAPQGEAFSLESCQNWL
PGEPHPATAEHCVRLGPTGWCNTDLCSAPHSYVCELQPGGPVQDAENLLVGAPSGDLQGPLT
PLAQQDGLSAPHEPVEVMVFPGIRLSREAFLTTAEFGTQELRRPAQLRLQVYRLLSTAGTPE
NGSEPESRSPDNRTQLAPACMPGGRWCPGANICLPLDASCHPQACANGCTSGPGLPGAPYAL
WREFLFSVPAGPPAQYSVTLHGQDVLMLPGDLVGLQHDAGPGALLHCSPAPGHPGPRAPYLS
ANASSWLPHLPAQLEGTWACPACALRLLAATEQLTVLLGLRPNPGLRLPGRYEVRAEVGNGV
SRHNLSCSFDVVSPVAGLRVIYPAPRDGRLYVPTNGSALVLQVDSGANATATARWPGGSVSA
RFENVCPALVATFVPGCPWETNDTLFSVVALPWLSEGEHVVDVVVENSASRANLSLRVTAEE
PICGLRATPSPEARVLQGVLVRYSPVVEAGSDMVFRWTINDKQSLTFQNVVENVIYQSAAVF
KLSLTASNHVSNVTVNYNVTVERMNRMQGLQVSTVPAVLSPNATLALTAGVLVDSAVEVAFL
WTFGDGEQALHQFQPPYNESFPVPDPSVAQVLVEHNVMHTYAAPGEYLLTVLASNAFENLTQ
QVPVSVRASLPSVAVGVSDGVLVAGRPVTFYPHPLPSPGGVLYTWDFGDGSPVLTQSQPAAN
HTYASRGTYHVRLEVNNTVSGAAAQADVRVFEELRGLSVDMSLAVEQGAPVVVSAAVQTGDN
ITWTFDMGDGTVLSGPEATVEHVYLRAQNCTVTVGAASPAGHLARSLHVLVFVLEVLRVEPA
ACIPTQPDARLTAYVTGNPAHYLFDWTFGDGSSNTTVRGCPTVTHNFTRSGTFPLALVLSSR
VNRAHYFTSICVEPEVGNVTLQPERQFVQLGDEAWLVACAWPPFPYRYTWDFGTEEAAPTRA
RGPEVTFIYRDPGSYLVTVTASNNISAANDSALVEVQEPVLVTSIKVNGSLGLELQQPYLFS
AVGRGRPASYLWDLGDGGWLEGPEVTHAYNSTGDFTVRVAGWNEVSRSEAWLNVTVKRRVRG
LVVNASRTVVPLNGSVSFSTSLEAGSDVRYSWVLCDRCTPIPGGPTISYTFRSVGTFNIIVT
AENEVGSAQDSIFVYVLQLIEGLQVVGGGRYFPTNHTVQLQAVVRDGTNVSYSWTAWRDRGP
ALAGSGKGFSLTVLEAGTYHVQLRATNMLGSAWADCTMDFVEPVGWLMVAASPNPAAVNTSV
TLSAELAGGSGVVYTWSLEEGLSWETSEPFTTHSFPTPGLHLVTMTAGNPLGSANATVEVDV
QVPVSGLSIRASEPGGSFVAAGSSVPFWGQLATGTNVSWCWAVPGGSSKRGPHVTMVFPDAG
TFSIRLNASNAVSWVSATYNLTAEEPIVGLVLWASSKVVAPGQLVHFQILLAAGSAVTERLQ
VGGANPEVLPGPRFSHSFPRVGDHVVSVRGKNHVSWAQAQVRIVVLEAVSGLQVPNCCEPGI
ATGTERNFTARVQRGSRVAYAWYFSLQKVQGDSLVILSGRDVTYTPVAAGLLEIQVRAFNAL
GSENRTLVLEVQDAVQYVALQSGPCFTNRSAQFEAATSPSPRRVAYHWDFGDGSPGQDTDEP
RAEHSYLRPGDYRVQVNASNLVSFFVAQATVTVQVLACREPEVDVVLPLQVLMRRSQRNYLE
AHVDLRDCVTYQTEYRWEVYRTASCQRPGRPARVALPGVDVSRPRLVLPRLALPVGHYCEVF
VVSFGDTPLTQSIQANVTVAPERLVPIIEGGSYRVWSDTRDLVLDGSESYDPNLEDGDQTPL
SFHWACVASTQREAGGCALNFGPRGSSTVTIPRERLAAGVEYTFSLTVWKAGRKEEATNQTV
LIRSGRVPIVSLECVSCKAQAVYEVSRSSYVYLEGRCLNCSSGSKRGRWAARTFSNKTLVLD
ETTTSTGSAGMRLVLRRGVLRDGEGYTFTLTVLGRSGEEEGCASIRLSPNRPPLGGSCRLFP
LGAVHALTTKVHFECTGWHDAEDAGAPLVYALLLRRCRQGHCEEFCVYKGSLSSYGAVLPPG
FRPHFEVGLAVVVQDQLGAAVVALNRSLAITLPEPNGSATGLTVWLHGLTASVLPGLLRQAD
PQHVIEYSLALVTVLNEYERALDVAAEPKHERQHRAQIRKNITETLVSLRVHTVDDIQQIAA
ALAQCMGPSRELVCRSCLKQTLHKLEAMMLILQAETTAGTVTPTAIGDSILNITGDLIHLAS
SDVRAPQPSELGAESPSRMVASQAYNLTSALMRILMRSRVLNEEPLTLAGEEIVAQGKRSDP
RSLLCYGGAPGPGCHFSIPEAFSGALANLSDVVQLIFLVDSNPFPFGYISNYTVSTKVASMA
FQTQAGAQIPIERLASERAITVKVPNNSDWAARGHRSSANSANSVVVQPQASVGAVVTLDSS
NPAAGLHLQLNYTLLDGHYLSEEPEPYLAVYLHSEPRPNEHNCSASRRIRPESLQGADHRPY
TFFISPGSRDPAGSYHLNLSSHFRWSALQVSVGLYTSLCQYFSEEDMVWRTEGLLPLEETSP
RQAVCLTRHLTAFGASLFVPPSHVRFVFPEPTADVNYIVMLTCAVCLVTYMVMAAILHKLDQ
LDASRGRAIPFCGQRGRFKYEILVKTGWGRGSGTTAHVGIMLYGVDSRSGHRHLDGDRAFHR
NSLDIFRIATPHSLGSVWKIRVWHDNKGLSPAWFLQHVIVRDLQTARSAFFLVNDWLSVETE
ANGGLVEKEVLAASDAALLRFRRLLVAELQRGFFDKHIWLSIWDRPPRSRFTRIQRATCCVL
LICLFLGANAVWYGAVGDSAYSTGHVSRLSPLSVDTVAVGLVSSVVVYPVYLAILFLERMSR
SKVAGSPSPTPAGQQVLDIDSCLDSSVLDSSFLTFSGLHAEQAFVGQMKSDLFLDDSKSLVC
WPSGEGTLSWPDLLSDPSIVGSNLRQLARGQAGHGLGPEEDGFSLASPYSPAKSFSASDEDL
IQQVLAEGVSSPAPTQDTHMETDLLSSLSSTPGEKTETLALQRLGELGPPSPGLNWEQPQAA
RLSRTGLVEGLRKRLLPAWCASLAHGLSLLLVAVAVAVSGWVGASFPPGVSVAWLLSSSASF
LASFLGWEPLKVLLEALYFSLVAKRLHPDEDDTLVESPAVTPVSARVPRVRPPHGFALFLAK
EEARKVKRLHGMLRSLLVYMLFLLVTLLASYGDASCHGHAYRLQSAIKQELHSRAFLAITRS
EELWPWMAHVLLPYVHGNQSSPELGPPRLRQVRLQEALYPDPPGPRVHTCSAAGGFSTSDYD
VGWESPHNGSGTWAYSAPDLLGAWSWGSCAVYDSGGYVQELGLSLEESRDRLRFLQLHNWLD
NRSRAVFLELTRYSPAVGLHAAVTLRLEFPAAGRALAALSVRPFALRRLSAGLSLPLLTSVC
LLLFAVHFAVAEARTWHREGRWRVLRLGAWARWLLVALTAATALVRLAQLGAADRQWTRFVR
GRPRRFTSFDQVAQLSSAARGLAASLLFLLLVKAAQQLRFVRQWSVFGKTLCRALPELLGVT
LGLVVLGVAYAQLAILLVSSCVDSLWSVAQALLVLCPGTGLSTLCPAESWHLSPLLCVGLWA
LRLWGALRLGAVILRWRYHALRGELYRPAWEPQDYEMVELFLRRLRLWMGLSKVKEFRHKVR
FEGMEPLPSRSSRGSKVSPDVPPPSAGSDASHPSTSSSQLDGLSVSLGRLGTRCEPEPSRLQ
AVFEALLTQFDRLNQATEDVYQLEQQLHSLQGRRSSRAPAGSSRGPSPGLRPALPSRLARAS
RGVDLATGPSRTPLRAKNKVHPSST,

(UniProtKB Accession No. 8161-1; (PKD1_HUMAN, length of 4,303 amino acids, molecular mass (Da) of 462,529). Exemplary alternative isoforms include:
UniProtKB Accession No. P98161-2; (length of 4,292 amino acids, molecular mass (Da) of 461,365), which differs from P98161-1 by:
   2497-2507: GWHDAEDAGAP (SEQ ID NO:3)→A; and
   3390-3390: Missing.
UniProtKB Accession No. P98161-3; (length of 4,302 amino acids, molecular mass (Da) of 462,401), which differs from P98161-1 by:
   3390-3390: Missing.

PC1 is cleaved at sites in both its N- and C-terminal domains (Chapin and Caplan, J. Cell Biol., 191, 701-710 (2010)). The C-terminal tail (CTT), which is also referred to as a PC1-CTT, is the final 200 amino acid of PC1. A consensus sequence for PC1-CTT is represented with bold and italics in SEQ ID NO:2:

(SEQ ID NO: 1)
VILRWRYHALRGELYRPAWEPQDYEMVELFLRRLRLWMGLSKVKEFRHKVR

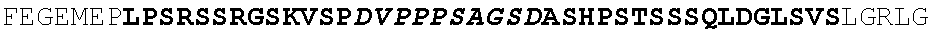

TRCEPEPSRLQAVFEALLTOFDRLNQATEDVYQLEQQLHSLQGRRSSRAPA
GSSRGPSPGLRPALPSRLARASRGVDLATGPSRTPLRAKNKVHPSST.

It has been discovered that PC1-CTT is sufficient to rescue phenotype(s) associated with ADPKD in a mouse model. Thus, compositions and methods for treatment of ADPKD are provided. The compositions typically are, or include, PC1-CTT (SEQ ID NO:1) or a functional fragment or variant thereof, or a nucleic acid encoding the same. Functional fragments and variants can be, for example, any number of amino acids sufficient to rescue one or more phenotypes of ADPKD or a mouse model thereof. The data below also supports the conclusions that PC1-CTT mitigates symptoms of ADPKD by interacting with nicotinamide nucleotide transhydrogenase (NNT). Thus, preferably the functional fragments maintain the ability to interact with, e.g., co-immunoprecipitate, NNT. In some embodiments, the fragment is between about 10 amino acids and about 195 amino acids of SEQ ID NO:1, or any subrange thereof, or any specific integer number of amino acids therebetween, including, but not limited to 20, 25, 50, 75, 100, 125, 150, or 175 amino acids. Variants can have, for example, at least 65%, 70%, 75%, 80%, 85%, 90%, 95%, or 100% sequence identity to SEQ ID NO:1, or a functional fragment thereof. Preferably variants can mitigate one or more symptoms of ADPKD and/or optionally, but preferably maintain the ability to interact with, e.g., co-immunoprecipitate, NNT.

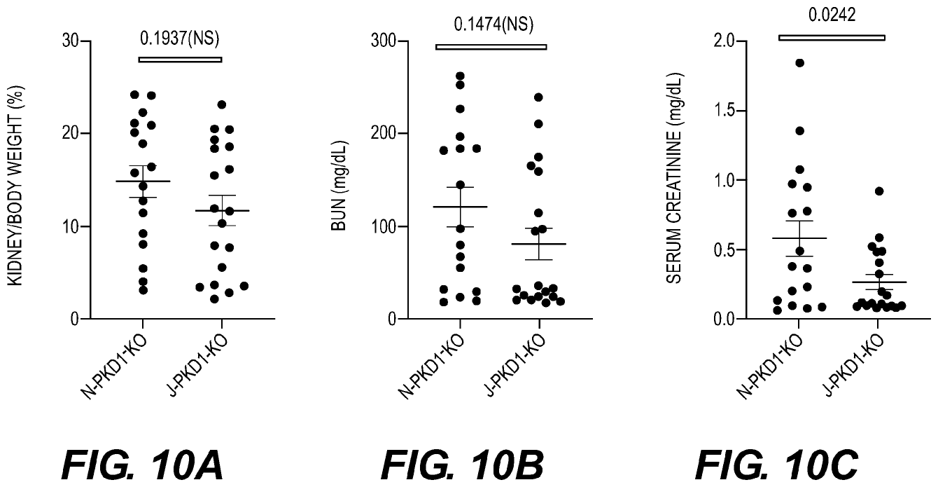

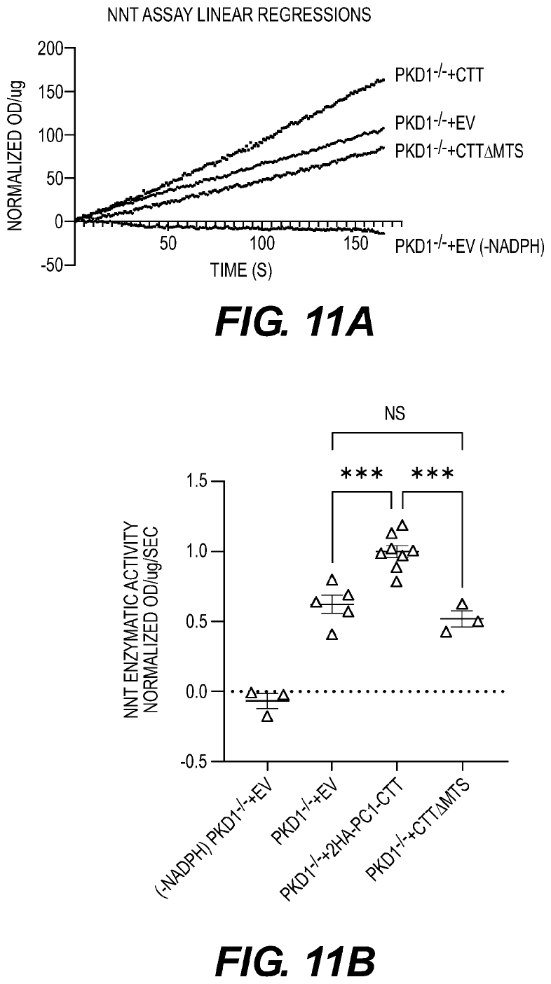

Results presented herein, e.g., FIGS. 11A and 11B confirm that PC1-CTT (i.e., SEQ ID NO:1) accumulates within the mitochondria. Transient transfection of PC1-CTT construct in Pkd1^{KO/KO} mouse cells reveals a significant increase in NNT enzymatic activity as compared to that of an empty expression vector of a mutant PC1-CTT construct lacking the the mitochondrial localization sequence (e.g., aa 4134-4154 (of SEQ ID NO:2)) (also referred to as deltaMTS). It is thus believed that having a mitochondrial localization signal, and perhaps the internal sequence LRRLRLWMGLSKVKEFRHKVR (SEQ ID NO:98) (wavy underlining in SEQ ID NO:1), or MVELFLRRLRLWMGLSKVKEFRHKVR (SEQ ID NO:99), is important for the biological activity of PC1-CTT. Thus, in some embodiments, the PC1-CTT polypeptide includes SEQ ID NO:98 or SEQ ID NO:99 or a variant thereof with at least 65%, 70%, 75%, 80%, 85%, 90%, 95%, or 100% sequence identity thereto, preferably where substitutions are conservative substitutions. As discussed elsewhere herein, any of the PC1-CTT polypeptides can include a heterologous mitochondrial localization signal. Thus, in some embodiments the amino acid sequence of SEQ ID NO:98 or SEQ ID NO:99 of the PC1-CTT polypeptide is mutated or absent, and the PC1-CTT polypeptide further includes a heterologous mitochondrial localization signal which can, for example, replace SEQ ID NOS:98 or 99, and/or be appended to the N- and/or C-terminus and/or add or inserted elsewhere in the PC1-CTT polypeptide.

Examples of preferred variants include those that inactivate the PEST motif. A PEST motif is a peptide sequence that is rich in proline (P), glutamic acid (E), serine (S), and threonine (T). This sequence is associated with proteins that have a short intracellular half-life; therefore, it is believed that the PEST sequence acts as a signal peptide for protein degradation (Rogers, et al., Science, 234 (4774): 364-8 (1986)). Thus, it is believed that alteration of this motif can lead to increased half-life of the PC1-CTT protein. The PEST motif in SEQ ID NO:1 is contained within the sequence in bold (LPSRSSRGSKVSPDVPPPSAGSDASHPSTSSSQLDGLSVS (SEQ ID NO:88), and is believed to include the sequence in italics (DVPPPSAGSD) (SEQ ID NO:89) (Low, et al., Dev Cell, 10(1):57-69 (2006). doi: 10.1016/j.devce1.2005.12.005). Results show that mutations of amino acids D71 and D80 of SEQ ID NO:1 (corresponding to D4174 and D4183 of SEQ ID NO:2) to alanine (i.e., DD->AA) is sufficient to reduce degradation of full-length PC1 (Low, et al., Dev Cell, 10(1):57-69 (2006). doi: 10.1016/j.devce1.2005.12.005).

The PC1-CTT polypeptide may include additional amino acid residues from full-length PC1, but typically does not include the entire full-length protein. Overall, the probability of successfully expressing a soluble protein decreases considerably at molecular weights above ˜60 kDa. See, e.g., “Protein production and purification,” Nat Methods, 5(2): 135-146 (2008). doi: 10.1038/nmeth.f.202, which is specifically incorporated by references herein in its entirety. Thus, the PC1-CTT sequence is preferably no more than 1,000, 900, 800, 700, 600, 500, 400, 300, or 200 amino acids of full-length PC1, or any other specific integer between 1,000 and 200. For example, in some embodiments, the length of the fragment or variant sequence thereof from full-length PC1 that forms part or all of the PC1-CTT polypeptide is between about 10 and 1,000 amino acids of PC1, or a subrange thereof or any specific integer number therebetween. In some embodiments, the entire PC1-CTT polypeptide, including any optional heterologous sequence(s), is no more than 1,000, 900, 800, 700, 600, 500, 400, 300, or 200 amino acids, or any other specific integer between 1,000 and 200.

Preferably variants can mitigate one or more symptoms of ADPKD and/or optionally, but preferably, maintain the ability to interact with, e.g., co-immunoprecipitate, NNT.

### 2. Heterologous Sequences

Heterologous functional elements that can be associated with, linked, conjugated, or otherwise attached directly or indirectly to the PC1-CTT polypeptide sequence, a nucleic acid, and/or to a nanoparticle or other delivery vehicle. Such molecules include, but are not limited to, protein transduction domains, fusogenic peptides, targeting molecules, and sequences that enhance protein expression and/or isolation.

a. Protein Transduction Domains

In some embodiments, the PC1-CTT polypeptide, a nucleic acid, and/or to a nanoparticle or other delivery vehicle includes a protein transduction domain (PTD). As used herein, a “protein transduction domain” or PTD refers to a polypeptide, polynucleotide, carbohydrate, organic or inorganic compound that facilitates traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle.

In preferred embodiments, the protein transduction domain is a polypeptide. A protein transduction domain can be a polypeptide including positively charged amino acids. Thus, some embodiments include PTDs that are cationic or amphipathic. Protein transduction domains (PTD), also known as a cell penetrating peptides (CPP), are typically polypeptides including positively charged amino acids. PTA can be short basic peptide sequences such as those present in many cellular and viral proteins.

PTDs are known in the art, and include but are not limited to small regions of proteins that are able to cross a cell membrane in a receptor-independent mechanism (Kabouridis, P., Trends in Biotechnology (11):498-503 (2003)). Although several PTDs have been documented, the two most commonly employed PTDs are derived from TAT (Frankel and Pabo, Cell, 55(6):1189-93(1988)) protein of HIV and Antennapedia transcription factor from Drosophila, whose PTD is known as Penetratin (Derossi et al., J Biol Chem., 269(14):10444-50 (1994)). Exemplary protein transduction domains include polypeptides with 11 Arginine residues, or positively charged polypeptides or polynucleotides having 8-15 residues, preferably 9-11 residues.

Penetratin and other derivatives of peptides derived from antennapedia (Cheng, et al., Biomaterials, 32(26):6194-203 (2011) can also be used. Results show that penetratin in which additional Args are added, further enhances uptake and endosomal escape, and IKK NBD, which has an antennapedia domain for permeation as well as a domain that blocks activation of NFkB and has been used safely in the lung for other purposes (von Bismarck, et al., Pulmonary Pharmacology & Therapeutics, 25(3):228-35 (2012), Kamei, et al., Journal Of Pharmaceutical Sciences, 102(11):3998-4008 (2013)). The Antennapedia homeodomain is 68 amino acid residues long and contains four alpha helices. Penetratin is an active domain of this protein which consists of a 16 amino acid sequence derived from the third helix of Antennapedia. TAT protein consists of 86 amino acids and is involved in the replication of HIV-1. The TAT PTD consists of an 11 amino acid sequence domain (residues 47 to 57; YGRKKRRQRRR (SEQ ID NO:4)) of the parent protein that appears to be critical for uptake. Additionally, the basic domain Tat(49-57) or RKKRRQRRR (SEQ ID NO:5) has been shown to be a PTD. In the current literature TAT has been favored for fusion to proteins of interest for cellular import. Several modifications to TAT, including substitutions of Glutamine to Alanine, i.e., Q->A, have demonstrated an increase in cellular uptake anywhere from 90% (Wender et al., Proc Natl Acad Sci USA., 97(24):13003-8 (2000)) to up to 33 fold in mammalian cells. (Ho et al., Cancer Res., 61(2):474-7 (2001)).

The most efficient uptake of modified proteins was revealed by mutagenesis experiments of TAT-PTD, showing that an 11 arginine stretch was several orders of magnitude more efficient as an intercellular delivery vehicle. Therefore, PTDs can include a sequence of multiple arginine residues, referred to herein as poly-arginine or poly-ARG. In some embodiments the sequence of arginine residues is consecutive. In some embodiments the sequence of arginine residues is non-consecutive. A poly-ARG can include at least 7 arginine residues, more preferably at least 8 arginine residues, most preferably at least 11 arginine residues. In some embodiments, the poly-ARG includes between 7 and 15 arginine residues, more preferably between 8 and 15 arginine residues, or any specific integer therebetween. In some embodiments the poly-ARG includes between 7 and 15, more preferably between 8 and 15 consecutive arginine residues. An example of a poly-ARG is RRRRRRR (SEQ ID NO:6). Additional exemplary PTDs include but are not limited to, RQIKIWFQNRRMKWKK (SEQ ID NO:7), mTAT (HIV-1 (with histidine modification) HHHHRKKRRQRRRRHHHHH (SEQ ID NO:8) (Yamano, et al., J Control Release, 152:278-285 (2011)); bPrPp (Bovine prion) MVKSKIGSWILVLFVAMWS DVGLCKKRPKP (SEQ ID NO:9) (Magzoub, et al., Biochem Biophys Res Commun., 348:379-385 (2006)); and MPG (Synthetic chimera: SV40 Lg T. Ant.+HIV gb41 coat) GALFLGFLGAAGSTMGAWS QPKKKRKV (SEQ ID NO:10) (Endoh, et al., Adv Drug Deliv Rev., 61:704-709 (2009)).

Short, non-peptide polymers that are rich in amines or guanidinium groups are also capable of carrying molecules across biological membranes.

A “fusogenic peptide” is any peptide with membrane destabilizing abilities. In general, fusogenic peptides have the propensity to form an amphiphilic alpha-helical structure when in the presence of a hydrophobic surface such as a membrane. The presence of a fusogenic peptide induces formation of pores in the cell membrane by disruption of the ordered packing of the membrane phospholipids. Some fusogenic peptides act to promote lipid disorder and, in this way, enhance the chance of merging or fusing of proximally positioned membranes of two membrane enveloped particles of various nature (e.g. cells, enveloped viruses, liposomes). Other fusogenic peptides may simultaneously attach to two membranes, causing merging of the membranes and promoting their fusion into one. Examples of fusogenic peptides include a fusion peptide from a viral envelope protein ectodomain, a membrane-destabilizing peptide of a viral envelope protein membrane-proximal domain from the cytoplasmic tails.

Other fusogenic peptides often also contain an amphiphilic region. Examples of amphiphilic-region containing peptides include: melittin, magainin, the cytoplasmic tail of HIV1 gp41, microbial and reptilian cytotoxic peptides such as bomolitin 1, pardaxin, mastoparan, carboline, cecropin, entamoeba, and staphylococcal alpha-toxin; viral fusion peptides from (1) regions at the N terminus of the transmembrane (TM) domains of viral envelope proteins, e.g. HIV-1, SIV, influenza, polio, rhinovirus, and coxsackie virus; (2) regions internal to the TM ectodomain, e.g. semliki forest virus, sindbis virus, rota virus, rubella virus and the fusion peptide from sperm protein PH-30: (3) regions membrane-proximal to the cytoplasmic side of viral envelope proteins e.g. in viruses of avian leukosis (ALV), Feline immunodeficiency (FIV), Rous Sarcoma (RSV), Moloney murine leukemia virus (MoMuLV), and spleen necrosis (SNV).

Without being bound by theory, it is believed that following an initial ionic cell-surface interaction, some polypeptides containing a protein transduction domain are rapidly internalized by cells via lipid raft-dependent micropinocytosis. For example, transduction of a TAT-fusion protein was found to be independent of interleukin-2 receptor/raft-, caveolar- and clathrin-mediated endocytosis and phagocytosis (Wadia, et al., Nature Medicine, 10:310-315 (2004), and Barka, et al., J. Histochem. Cytochem., 48(11):1453-60 (2000)). Therefore, in some embodiments the polypeptides include an endosomal escape sequence that enhances escape of the polypeptide from macropinosomes. In some embodiments the endosomal escape sequence is part of, or consecutive with, the protein transduction domain. In some embodiments, the endosomal escape sequence is non-consecutive with the protein transduction domain. In some embodiments the endosomal escape sequence includes a portion of the hemagglutinin peptide from influenza (HA).

The efficiency of nanoparticle delivery systems can also be improved by the attachment of functional ligands to the NP surface. Potential ligands include, but are not limited to, small molecules, cell-penetrating peptides (CPPs), targeting peptides, antibodies or aptamers (Yu, et al., PLoS One., 6:e24077 (2011), Cu, et al., J Control Release, 156:258-264 (2011), Nie, et al., J Control Release, 138:64-70 (2009), Cruz, et al., J Control Release, 144:118-126 (2010)). Attachment of these moieties serves a variety of different functions, such as inducing intracellular uptake, endosome disruption, and delivery of the plasmid payload to the nucleus. There have been numerous methods employed to tether ligands to the particle surface. One approach is direct covalent attachment to the functional groups on PLGA NPs (Bertram, Acta Biomater. 5:2860-2871 (2009)). Another approach utilizes amphiphilic conjugates like avidin palmitate to secure biotinylated ligands to the NP surface (Fahmy, et al., Biomaterials, 26:5727-5736 (2005), Cu, et al., Nanomedicine, 6:334-343 (2010)). This approach produces particles with enhanced uptake into cells, but reduced pDNA release and gene transfection, which is likely due to the surface modification occluding pDNA release. In a similar approach, lipid-conjugated polyethylene glycol (PEG) is used as a multivalent linker of penetratin, a CPP, or folate (Cheng, et al., Biomaterials, 32:6194-6203 (2011)).

b. Targeting Signal or Domain

In some embodiments the PC1-CTT polypeptide, a nucleic acid, and/or to a nanoparticle or other delivery vehicle is modified to include one or more targeting signals or domains. The targeting signal can include a sequence of monomers that facilitates in vivo localization of the molecule. The monomers can be amino acids, nucleotide or nucleoside bases, or sugar groups such as glucose, galactose, and the like which form carbohydrate targeting signals. Targeting signals or sequences can be specific for a host, tissue, organ, cell, organelle, non-nuclear organelle, or cellular compartment. For example, in some embodiments the polynucleotide-binding polypeptide includes both a cell-specific targeting domain and an organelle specific targeting domain to enhance delivery of the polypeptide to a subcellular organelle of a specific cells type.

i. Organelle Targeting

In some embodiments, the targeting domain targets a subcellular organelle. Targeting of the disclosed polypeptides to organelles can be accomplished by modifying the disclosed compositions to contain specific organelle targeting signals. These sequences can target organelles, either specifically or non-specifically. In some embodiments the interaction of the targeting signal with the organelle does not occur through a traditional receptor: ligand interaction.

### Targeting the Mitochondria

The results in the experiments demonstrate that NNT activity in a cell line that lacks polycystin-1 expression can be substantially increased by expressing the polycystin-1 C-terminal tail, and this effect does not occur if the polycystin-1 C-terminal tail lacks the mitochondrial localization sequence. Therefore, in certain embodiments the composition specifically targets mitochondria. In some forms, the composition targets the inner mitochondrial membrane. In other forms, the composition targets the mitochondrial-associated endoplasmic reticulum membranes (MAMs). In some forms, the composition interacts with one or more proteins e.g., enzymes, to regulate cellular and mitochondrial dynamics. For example, the composition may interact with one or more proteins to modulate tubular/cyst cell proliferation, the metabolic profile, mitochondrial function, and the redox state.

Mitochondrial targeting agents may include a leader sequence of highly positively charged amino acids. This allows the protein to be targeted to the highly negatively charged mitochondria. Unlike receptor: ligand approaches that rely upon stochastic Brownian motion for the ligand to approach the receptor, the mitochondrial localization signal of some embodiments is drawn to mitochondria because of charge. Therefore, in some embodiments, the mitochondrial targeting agent is a protein transduction domain including but not limited to the protein transduction domains discussed in detail above.

Mitochondrial targeting agents also include short peptide sequences (Yousif, et al., Chembiochem., 10(13):2131 (2009), for example mitochondrial transporters-synthetic cell-permeable peptides, also known as mitochondria-penetrating peptides (MPPs), that are able to enter mitochondria. MPPs are typically cationic, but also lipophilic; this combination of characteristics facilitates permeation of the hydrophobic mitochondrial membrane. For example, MPPs can include alternating cationic and hydrophobic residues (Horton, et al., Chem Biol., 15(4):375-82 (2008)). Some MPPs include delocalized lipophilic cations (DLCs) in the peptide sequence instead of, or in addition to natural cationic amino acids (Kelley, et al., Pharm. Res., 2011 Aug. 11 [Epub ahead of print]). Other variants can be based on an oligomeric carbohydrate scaffold, for example attaching guanidinium moieties due to their delocalized cationic form (Yousif, et al., Chembiochem., 10(13):2131 (2009).

Mitochondrial targeting agents also include mitochondrial localization signals or mitochondrial targeting signals. Such sequences are known in the art, see for example, U.S. Pat. No. 8,039,587, which is specifically incorporated by reference herein in its entirety. The identification of the specific sequences necessary for translocation of a linked compound into a mitochondrion can be determined using predictive software known to those skilled in the art.

### Nuclear Localization Signals

The compositions disclosed herein can include one or more nuclear localization signals. Nuclear localization signals (NLS) are known in the art and include for example, SV 40 T antigen or a fragment thereof. The NLS can be simple cationic sequences of about 4 to about 8 amino acids, or can be bipartite having two interdependent positively charged clusters separated by a mutation resistant linker region of about 10-12 amino acids.

The PC1-CTT sequence includes a nuclear localization signal, which may be present or absent in the PC1-CTT polypeptide. The PC1-CTT polypeptide can also include an additional or alternative nuclear localization signal. Thus, the PC1-CTT polypeptide may include no nuclear localization signal, or the endogenous nuclear localization signal and/or one or more heterologous nuclear localization signals.

ii. Cell Targeting

The compositions disclosed herein can be modified to target a specific cell type or population of cells.

In one embodiment, the targeting signal binds to its ligand or receptor which is located on the surface of a target cell such as to bring the composition and cell membranes sufficiently close to each other to allow penetration of the composition into the cell.

In some embodiments, the targeting molecule is an antibody or antigen binding fragment thereof, an antibody domain, an antigen, a cell receptor, a cell surface receptor, a cell surface adhesion molecule, a viral envelope protein and a peptide selected by phage display that binds specifically to a defined cell.

Targeting a polypeptide of interest to specific cells can be accomplished by modifying the polypeptide of interest to express specific cell and tissue targeting signals. These sequences target specific cells and tissues. In some embodiments the interaction of the targeting signal with the cell does not occur through a traditional receptor: ligand interaction. The eukaryotic cell comprises a number of distinct cell surface molecules. The structure and function of each molecule can be specific to the origin, expression, character and structure of the cell. Determining the unique cell surface complement of molecules of a specific cell type can be determined using techniques well known in the art.

One skilled in the art will appreciate that the tropism of the proteins of interest described can be altered by changing the targeting signal. In one specific embodiment, compositions are provided that enable the addition of cell surface antigen specific antibodies to the composition for targeting the delivery of polynucleotide-binding polypeptide. Exemplary cell surface antigens are disclosed in Wagner et al., Adv Gen, 53:333-354 (2005) which is specifically incorporated by reference herein in its entirety.

It is known in the art that nearly every cell type in a tissue in a mammalian organism possesses some unique cell surface receptor or antigen. Thus, it is possible to incorporate nearly any ligand for the cell surface receptor or antigen as a targeting signal. For example, peptidyl hormones can be used a targeting moieties to target delivery to those cells which possess receptors for such hormones. Chemokines and cytokines can similarly be employed as targeting signals to target delivery of the complex to their target cells. A variety of technologies have been developed to identify genes that are preferentially expressed in certain cells or cell states and one of skill in the art can employ such technology to identify targeting signals which are preferentially or uniquely expressed on the target tissue of interest.

In particular embodiments, the target cells are kidney cells, optionally wherein the kidney cells are kidney epithelial cells. Examples include, but are not limited to, Cadherin 16 (CDH16) which expressed on the basolateral surfaces of most renal epithelial cells (Thomson and Aronson, Am J Physiol., 277(1) F146-56 (1999). doi:10.1152/ajprenal.1999.277.1.f146. PMID: 10409308. In another embodiment, the target is the V2 vasopressin receptor, which is expressed in principal cells of the renal collecting duct, the same cells targeted by Tolvaptan, the current small molecule therapy for PKD.

Other preferred target cells types include bile duct cholangiocytes and/or pancreatic duct cells, as cysts can form in both of these tissues in ADPKD.

c. Additional Sequences

The compositions can optionally include additional sequences or moieties, including, but not limited to linkers and purification tags.

In a preferred embodiment the purification tag is a polypeptide. Polypeptide purification tags are known in the art and include, but are not limited to His tags which typically include six or more, typically consecutive, histidine residues; FLAG tags, which typically include the sequence DYKDDDDK (SEQ ID NO:11); haemagglutinin (HA) for example, YPYDVP (SEQ ID NO:12); MYC tag for example ILKKATAYIL (SEQ ID NO:13) or EQKLISEEDL (SEQ ID NO:14). Methods of using purification tags to facilitate protein purification are known in the art and include, for example, a chromatography step wherein the tag reversibly binds to a chromatography resin.

Purifications tags can be N-terminal or C-terminal to a protein. The purification tags N-terminal to the recombinant protein can be separated from the polypeptide of interest at the time of the cleavage in vivo. Therefore, purification tags N-terminal to the recombinant protein can be used to remove the recombinant protein from a cellular lysate following expression and extraction of the expression or solubility enhancing amino acid sequence, but cannot be used to remove the polypeptide of interest. Purification tags C-terminal to the recombinant protein can be used to remove the polypeptide of interest from a cellular lysate following expression of the recombinant protein, but cannot be used to remove the expression or solubility enhancing amino acid sequence. Purification tags that are C-terminal to the expression or solubility enhancing amino acid sequence can be N-terminal to, C-terminal to, or incorporated within the sequence of the polypeptide of interest.

In some embodiments, the recombinant protein includes one or more linkers or spacers. The term “linker” as used herein includes, without limitation, peptide linkers. The peptide linker can be any size provided it does not interfere with the binding of the epitope by the variable regions. In some embodiments, the linker includes one or more glycine and/or serine amino acid residues. In some embodiments, the linker includes a glycine-glutamic acid di-amino acid sequence. For example, a linker can include 4-8 amino acids. In a particular embodiment, a linker includes the amino acid sequence GQSSRSS (SEQ ID NO:15). In another embodiment, a linker includes 15-20 amino acids, for example 18 amino acids. Other flexible linkers include, but are not limited to, the amino acid sequences Gly-Ser, Gly-Ser-Gly-Ser (SEQ ID NO:16), Ala-Ser, Gly-Gly-Gly-Ser (SEQ ID NO:17), (Gly₄-Ser)₂ (SEQ ID NO:18) and (Gly₄-Ser)₄ (SEQ ID NO:19), and (Gly-Gly-Gly-Gly-Ser)₃ (SEQ ID NO:20).

The linkers can be used to link or connect two domains, regions, or sequences of a fusion protein.

Molecular biology techniques have developed so that therapeutic proteins can be genetically engineered to be expressed by microorganisms. The gram negative bacterium, Escherichia coli, is a versatile and valuable organism for the expression of therapeutic proteins. Although many proteins with therapeutic or commercial uses can be produced by recombinant organisms, the yield and quality of the expressed protein are variable due to many factors. For example, heterologous protein expression by genetically engineered organisms can be affected by the size and source of the protein to be expressed, the presence of an affinity tag linked to the protein to be expressed, codon biasing, the strain of the microorganism, the culture conditions of microorganism, and the in vivo degradation of the expressed protein. Some of these problems can be mitigated by fusing the protein of interest to an expression or solubility enhancing amino acid sequence. Exemplary expression or solubility enhancing amino acid sequences include maltose-binding protein (MBP), glutathione S-transferase (GST), thioredoxin (TRX), NUS A, ubiquitin (Ub), and a small ubiquitin-related modifier (SUMO).

In some embodiments, the compositions disclosed herein include expression or solubility enhancing amino acid sequence. In some embodiments, the expression or solubility enhancing amino acid sequence is cleaved prior administration of the composition to a subject in need thereof. The expression or solubility enhancing amino acid sequence can be cleaved in the recombinant expression system, or after the expressed protein in purified.

### B. Nucleic Acids

### 1. Isolated Nucleic Acid Molecules

Isolated nucleic acid sequences encoding PC1-CTT polypeptides are disclosed. Also provided are nucleic acid encoding therapeutic polypeptides for the treatment of other diseases and disorders characterized by high levels of mTOR signaling.

As used herein, “isolated nucleic acid” refers to a nucleic acid that is separated from other nucleic acid molecules that are present in a mammalian genome, including nucleic acids that normally flank one or both sides of the nucleic acid in a mammalian genome. An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence encoding a PC1-CTT polypeptide. Thus, nucleic acids encoding SEQ ID NOS:1 and 2, and fragments and variants thereof, in sense and antisense, and in single stranded and double stranded forms, are provided.

Also provided are nucleic acid encoding therapeutic polypeptides for the treatment of other diseases and disorders characterized by high levels of mTOR signaling, in sense and antisense, and in single stranded and double stranded forms.

Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxy uridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxy phosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxy phosphate backbone can be replaced with, for example, a phosphorothioate or phosphorodithioate backbone, a phosphoroamidite, or an alkyl phosphotriester backbone.

### 2. Vectors

The application also relates to vectors including an isolated polynucleotide encoding an PC1-CTT polypeptide and/or therapeutic polypeptides for the treatment of other diseases and disorders characterized by high levels of mTOR signaling. As used herein, a “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). A vector can be a DNA vector or an RNA vector. In some embodiments, a vector is a DNA plasmid. One of ordinary skill in the art can construct a vector of the application through standard recombinant techniques in view of the present disclosure.

A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

Vectors can contain a variety of regulatory sequences. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

In some embodiments, the vector is a non-viral vector. Examples of non-viral vectors include, but are not limited to, DNA plasmids, bacterial artificial chromosomes, yeast artificial chromosomes, bacteriophages, etc. Examples of non-viral vectors include, but are not limited to, RNA replicon, mRNA replicon, modified mRNA replicon or self-amplifying mRNA, closed linear deoxyribonucleic acid, e.g., a linear covalently closed DNA, e.g., a linear covalently closed double stranded DNA molecule. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically comprise an origin of replication, a multiple cloning site, and a selectable marker, which for example, can be an antibiotic resistance gene. Examples of suitable DNA plasmids that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC®. complete baculovirus expression system (Thermo Fisher Scientific), which can be used for production and/or expression in insect cells; pcDNA™. or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNA™, pcDNA3™, pVAX, pVAX-1, ADVAX, NTC8454, etc. In some embodiments, an expression vector is based on pVAX-1, which can be further modified to optimize protein expression in mammalian cells. pVAX-1 is a commonly used plasmid in DNA vaccines, and contains a strong human immediate early cytomegalovirus (CMV-IE) promoter followed by the bovine growth hormone (bGH)-derived polyadenylation sequence (pA). pVAX-1 further contains a pUC origin of replication and a kanamycin resistance gene driven by a small prokaryotic promoter that allows for bacterial plasmid propagation.

The vector can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, arenavirus viral vectors, replication-deficient arenavirus viral vectors or replication-competent arenavirus viral vectors, bi-segmented or tri-segmented arenavirus, infectious arenavirus viral vectors, nucleic acids which include an arenavirus genomic segment wherein one open reading frame of the genomic segment is deleted or functionally inactivated (and replaced by a nucleic acid encoding a PC1-CTT polypeptide or another therapeutic polypeptide as described herein), arenavirus such as lymphocytic choriomeningitis virus (LCMV), e.g., clone 13 strain or MP strain, and arenavirus such as Junin virus e.g., Candid #1 strain, etc.

In some embodiments, the viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other embodiments, an adenovirus vector is a rhAd vector, e.g. rhAd51, rhAd52 or rhAd53. A recombinant viral vector can be prepared using methods known in the art in view of the present disclosure. For example, in view of the degeneracy of the genetic code, several nucleic acid sequences can be designed that encode the same polypeptide. A polynucleotide encoding a PC1-CTT polypeptide or other therapeutic polypeptide of the application can optionally be codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

The vectors, e.g., a DNA plasmid or a viral vector (particularly an adenoviral vector), can include any regulatory elements to establish conventional function(s) of the vector, including but not limited to replication and expression of the PC1-CTT polypeptide or other therapeutic polypeptide encoded by the polynucleotide sequence of the vector.

### 3. Regulatory Elements

Any of the disclosed nucleic acids, including RNAs and DNAs such as DNA vectors can include one or more regulatory elements. Regulatory elements include, but are not limited to, a promoter, an enhancer, a polyadenylation signal, translation stop codon, a ribosome binding element, a transcription terminator, selection markers, origin of replication, etc. An isolated include acid can be, and a vector can include, one or more expression cassettes. An “expression cassette” is part of a nucleic acid such as a vector that directs the cellular machinery to make RNA and protein. An expression cassette typically includes three components: a promoter sequence, an open reading frame, and a 3-untranslated region (UTR) optionally including a polyadenylation signal. An open reading frame (ORF) is a reading frame that contains a coding sequence of a protein of interest (e.g., PC1-CTT polypeptide, other therapeutic polypeptide, etc.) from a start codon to a stop codon. Regulatory elements of the expression cassette can be operably linked to a polynucleotide sequence encoding a PC1-CTT polypeptide or other therapeutic polypeptide.

As used herein, the term “operably linked” is to be taken in its broadest reasonable context, and refers to a linkage of polynucleotide (or polypeptide, etc.) elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

a. Promotors

The disclosed nucleic acids, including vectors, can include a promoter sequence, preferably within an expression cassette, to control expression of a PC1-CTT polypeptide or other therapeutic polypeptide of interest. The term “promoter” is used in its conventional sense, and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding a PC1-CTT polypeptide within an expression cassette.

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metallothionein.

A promoter can also be a tissue specific promoter, such as a kidney specific promoter, preferably a kidney epithelial cell promoter, which can be natural or synthetic. Examples include, but are not limited to, the CDH 16 promoter, which is mostly kidney specific (it is also expressed in the thyroid) (Igarashi, et al., Am J Physiol., 277(4):F599-610 (1999). doi: 10.1152/ajprenal.1999.277.4.F599. PMID: 10516285.); the Pax-8 promoter, which is also expressed primarily in the kidney as well as in the thyroid (Dehbi, et al., EMBO J., 15(16):4297-306 (1996) PMID: 8861958); the aquaporin 2 promoter, which drives expression specifically in principal cells of the renal collecting duct (which are the target of Tolvaptan) (Stricklett, et al., Exp Nephrol., 7(1):67-74 (1999). doi: 10.1159/000020587. PMID: 9892817.), and kidney tubule-specific promoters in association with gene delivery viral vectors (Watanabe, et al., PloS one, vol. 12,3 e0168638 (2017), doi:10.1371/journal.pone.0168638).

In some embodiments, the promoter is a strong eukaryotic promoter, such as cytomegalovirus immediate early (CMV-IE) promoter.

b. Other Expression Control Elements

The disclosed nucleic acids, including vectors, can include additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a PC1-CTT polypeptide or other therapeutic polypeptide within an expression cassette of the vector. Enhancer sequences are regulatory DNA sequences that, when bound by transcription factors, enhance the transcription of an associated gene. An enhancer sequence is preferably located upstream of the polynucleotide sequence encoding a PC1-CTT polypeptide or other therapeutic polypeptide, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human beta-globin polyadenylation signal. Preferably, a polyadenylation signal is a bovine growth hormone (bGH) polyadenylation signal or a SV40 polyadenylation signal.

Any enhancer sequence known to those skilled in the art in view of the present disclosure can be used. For example, an enhancer sequence can be a human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit beta-globin intron, or any combination thereof. Preferably, an enhancer sequence is a composite sequence of three consecutive elements of the untranslated R-U5 domain of HTLV-1 LTR, rabbit beta-globin intron, and a splicing enhancer, which is referred to herein as “a triple enhancer sequence.”

A vector can include a polynucleotide sequence encoding a signal peptide sequence. Preferably, the polynucleotide sequence encoding the signal peptide sequence is located upstream of the polynucleotide sequence encoding a PC1-CTT polypeptide or other therapeutic polypeptide. Signal peptides typically direct localization of a protein, facilitate secretion of the protein from the cell in which it is produced, and/or improve expression the therapeutic polypeptide when expressed from the vector, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as the Ig heavy chain gamma signal peptide SPIgG or the Ig heavy chain epsilon signal peptide SPIgE.

A vector, such as a DNA plasmid, can also include a bacterial origin of replication and an antibiotic resistance expression cassette for selection and maintenance of the plasmid in bacterial cells, e.g., E. coli. Bacterial origins of replication and antibiotic resistance cassettes can be located in a vector in the same orientation as the expression cassette encoding a PC1-CTT polypeptide or other therapeutic polypeptide, or in the opposite (reverse) orientation. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC.

Expression cassettes for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest, e.g., a PC1-CTT polypeptide or other therapeutic polypeptide. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kan^{r}), ampicillin resistance gene (Amp^{r}), and tetracycline resistance gene (Tet^{r}), as well as genes conferring resistance to chloramphenicol, bleomycin, spectinomycin, carbenicillin, etc.

An expression vector can include a tag sequence, such as those discussed above.

c. mTORC1-Dependent Translation Control

In some embodiments, the nucleic acids include an mTORC1-dependent translation control sequence. Cell growth is a highly regulated process that fluctuates with changes in nutrient and other growth signals. In eukaryotes, the mTOR Complex 1 (mTORC1) signaling pathway is at the heart of a system that orchestrates this program. A major mTORC1 function is to control general protein synthesis, but also the selective translation of a family of mRNAs that are defined by a 5′ terminal oligopyrimidine (TOP) motif. The TOP motif itself is a series of 4-15 pyrimidines that are adjacent to the 5′ terminal cap structure and are necessary and sufficient to render an mRNA subject to this mTORC1 mechanism.

Typically, the motif includes a C at the +1 position followed by a series of 4 or more pyrimidines. The +1 C is important, while sequences of greater than 4 pyrimidines have little effect on translation regulation in this context. Preferably, if expressed from a plasmid, the +2 position is a U/T, as this is designed for higher levels of transcription.

See, also e.g., Meyuhas, “Synthesis of the translational apparatus is regulated at the translational level,” Eur J Biochem, 267(21):6321-30 (2000). doi: 10.1046/j.1432-1327.2000.01719.x., which is specifically incorporated by reference herein in its entirety).

Comparative analysis of the first 20 nucleotides of 32 mammalian TOP mRNAs indicates the following structural features of the translational cis-regulatory element (Table 1).

TABLE 1
The 5′ terminus of mammalian TOP mRNAs.

| | | | 5′ | | | |
|---|---|---|---|---|---|---|
| | | | TOP | C/U | 5′ UTR | Accession |
| Protein | Sequence | | (nt) | ratio | (nt) | Number |
| | +1 +10 +20 | | | | | |
| | I I I | | | | | |
| m rpS4 | CUCUUUCCGUUCCUAGCGCA | (SEQ ID NO: 21) | 8 | 4/4 | 23 | L24371 |
| h rpS4Y | CUCUUCCGUCGCAGAGUUUC | (SEQ ID NO: 22) | 7 | 4/3 | 23 | L24370 |
| h rpS4X | CCUCUUUCCUUGCCUAACGC | (SEQ ID NO: 23) | 11 | 5/6 | 24 | L24369 |
| h rpS6 | CCUCUUUUCCGUGGCGCCUC | (SEQ ID NO: 24) | 10 | 5/5 | 42 | X67309, |
| | | | | | | M77232 |
| h rps8 | CUCUUUCCAGCCAGCGCCGA | (SEQ ID NO: 25) | 8 | 4/4 | 23 | X67247 |
| m rpS11 | CCUUUCUCCGGCGGCCGGGA | (SEQ ID NO: 26) | 9 | 5/4 | 23 | AB028894 |
| h rpS11 | CUUUUUUUCAGGCGGCCGGG | (SEQ ID NO: 27) | 9 | 2/7 | 24 | AB028893 |
| h rpS14 | CUCUUUCCGGUGUGGAGUCU | (SEQ ID NO: 28) | 8 | 4/4 | 37 | M13934 |
| h rpS15 | CUUUUCCCAGCAGCCGCCAA | (SEQ ID NO: 29) | 8 | 4/4 | 21 | D11388 |
| m rpS16 | CCUUUUCCGGUCGCGGCGCU | (SEQ ID NO: 30) | 8 | 4/4 | 52 | M11408 |
| h rpS17 | CCUCUUUUACCAAGGACCCG | (SEQ ID NO: 31) | 8 | 3/5 | 25 | M18000 |
| h rpS24 | CUUUUCCUCCUUGGCUGUCU | (SEQ ID NO: 32) | 12 | 5/7 | 37 | U12202 |
| m rpP0 | CUUCUCUCGCCAGGCGUCCU | (SEQ ID NO: 33) | 8 | 4/4 | 77 | X15267 |
| r rpP2 | CUUUCGCCGCCGGACGCCGC | (SEQ ID NO: 34) | 5 | 2/3 | 58 | X55153 |
| m rpL7 | CUCUCUUCUUUUCCGGCUGG | (SEQ ID NO: 35) | 14 | 6/8 | 24 | M29015 |
| m rpL7a | CUUUCUUUCUCCAGCAGCCG | (SEQ ID NO: 36) | 12 | 5/7 | 26 | X54067 |
| h rpL7a | CUCUCUCCUCCCGCCGCCCA | (SEQ ID NO: 37) | 12 | 8/4 | 22 | X61923 |
| m rpL13a | CUUUCCCAGGCGGCUGCCGA | (SEQ ID NO: 38) | 7 | 3/4 | 22 | X51528 |
| h rpL13a | CUUUUCCAAGCGGCUGCCGA | (SEQ ID NO: 39) | 7 | 3/4 | 27 | AB028893 |
| m rpL30 | CCUUUCUCGCUCCCCGGCCG | (SEQ ID NO: 40) | 8 | 4/4 | 38 | K02928 |
| m rpL32 | CUUCUUCCUCGGCGCUGCCU | (SEQ ID NO: 41) | 10 | 5/5 | 52 | K02060 |
| r rpL35a | CUCUUUCUGCCAUCUUGGCG | (SEQ ID NO: 42) | 8 | 3/5 | 33 | X05704, |
| | | | | | | 5, 6 |
| h eEF1A | CUUUUUCGCAACGGGUUUGC | (SEQ ID NO: 43) | 7 | 2/5 | 63 | J04617 |
| h eEF1B | CUUUUUCCUCUCUUCAGCGT | (SEQ ID NO: 44) | 15 | 6/9 | 84 | D28350 |
| ch eEF2 | CUCUUCCGCCGCAGCCGCCG | (SEQ ID NO: 45) | 7 | 4/3 | 83 | J03200 |
| h PABP | CCCCUUCUCCCCGGCGGUUA | (SEQ ID NO: 46) | 12 | 9/3 | 505 | U68093 |
| m P40 | CUUUUUUCGCGCUACCCGGG | (SEQ ID NO: 47) | 8 | 2/6 | 79 | J02870, |
| | | | | | | X06406 |
| h QM | CUCUUUCCCUUCGGUGUGGU | (SEQ ID NO: 48) | 12 | 6/6 | 143 | U37218, |
| | | | | | | D28410 |
| h B23 | CUUUCCCUGGUGUGAUUCCG | (SEQ ID NO: 49) | 8 | 4/4 | 100 | U89309, |
| | | | | | | D28343 |
| m P23 | CUUUUUUCCGCCCGCUCCCC | (SEQ ID NO: 50) | 9 | 3/6 | 101 | X06407 |
| rb P23 | CUUUUCCGCCCGCUCCCCCC | (SEQ ID NO: 51) | 7 | 3/4 | 116 | AJ225898 |
| h P23 | CUUUUCCGCCCGCUCCCCCC | (SEQ ID NO: 52) | 7 | 4/3 | 90 | D28408, |

| Position | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| A | 0 | 0 | 0 | 0 | 0 | 0 | 0 | 2 | 4 | 2 | 1 | 4 | 5 | 2 | 4 | 5 | 1 | 0 | 1 | 8 |
| C | 32 | 7 | 10 | 6 | 6 | 12 | 21 | 17 | 13 | 16 | 13 | 15 | 10 | 10 | 17 | 6 | 16 | 19 | 16 | 8 |
| G | 0 | 0 | 0 | 0 | 0 | 1 | 0 | 5 | 10 | 9 | 7 | 10 | 12 | 16 | 6 | 15 | 10 | 7 | 12 | 9 |
| U | 0 | 25 | 22 | 26 | 26 | 19 | 11 | 8 | 5 | 5 | 10 | 3 | 5 | 5 | 5 | 6 | 5 | 6 | 3 | 7 |
| Consensus | C | U/c | U/c | U/c | U/c | U/c | C/U | C | C/G | C | C/U | C/G | G/C | G/C | C | G | C/G | C | C/G | N |
| (SEQ ID | | | | | | | | | | | | | | | | | | | | |
| NO: 87) | | | | | | | | | | | | | | | | | | | | |

Sequences presented in this table represent only mRNAs whose 5′ termini has been rigorously determined by primer extension,S1 nuclease mapping or comparative analysis of processed pseudogenes.
ch, Chinese hamster; m, mouse; r, rat; rb, rabbit; h, human.
N, any nucleotide.

(Table 1, adapted from Meyuhas, “Synthesis of the translational apparatus is regulated at the translational level,” Eur J Biochem, 267(21):6321-30 (2000). doi: 10.1046/j.1432-1327.2000.01719.x, (Table 1 provides the first twenty 5′ nucleotides for 32 TOP sequences SEQ ID NOS:21-52 (32 total), and the TOP motifs (underlined portions) thereof (below, and as SEQ ID NOS:56-81 (32 total)), as well as a consensus TOP motif sequence (SEQ ID NO:87). The TOP motifs (underlined portions of SEQ ID NOs:21-52) are listed as follows:

(SEQ ID NO: 56)
CUCUUUCC, CUCUUCC, CCUCUUUCCUU,
(SEQ ID NO: 57)
CCUCUUUUCC,
(SEQ ID NO: 65)
CUCUUUCC, CCUUUCUCC, CUUUUUUUC, CUCUUUCC,
CUUUUCCC, CCUUUUCC, CCUCUUUU, CUUUUCCUCCUU,
(SEQ ID NO: 68)
CUUCUCUC, CUUUC, CUCUCUUCUUUUCC,
(SEQ ID NO: 69)
CUUUCUUUCUCC,
(SEQ ID NO: 70)
CUCUCUCCUCCC,
(SEQ ID NO: 74)
CUUUCCC, CUUUUCC, CCUUUCUC, CUUCUUCCUC,
(SEQ ID NO: 77)
CUCUUUCU, CUUUUUC, CUUUUUCCUCUCUUC,
(SEQ ID NO: 79)
CUCUUCC, CCCCUUCUCCCC,
(SEQ ID NO: 81)
CUUUUUUC, CUCUUUCCCUUC, CUUUCCCU, CUUUUUUCC,
CUUUUCC,
and
CUUUUCC.

Meyuhas provides the following preferred criteria of a TOP motif and TOP motif-containing sequences:
(a) It starts with a C residue at the cap site, which is followed by an uninterrupted stretch of 4-14 pyrimidines. It should be emphasized that only about 17% of mammalian transcripts start at a C residue at the cap site, whereas most others start at an A residue.
(b) The composition of the 5′ TOP, although varying among TOP mRNAs even within a species, generally maintains a similar proportion of C and U residues.
(c) The 5′ TOP motif is followed by a CG-rich sequence.
(d) These mRNAs possess 5′ UTRs which vary in length from 21 to 505 nt, but are devoid of upstream AUGs.

Thoreen, et al., “A unifying model for mTORC1-mediated regulation of mRNA translation”, Nature, 485:109-13 (2012) (which is specifically incorporated by reference in its entirety including the supplemental materials), identified additional/alternative criteria including previously unrecognized TOP motifs and mTOR regulated sequences with a stretch of pyrimidines that was near but did not begin at the most frequent transcriptional start site (TSS). Results show that ten nucleotides surrounding the predominant transcriptional start site (TSS) in the mRNAs most suppressed by mTOR inhibition were still highly enriched for pyrimidines, leading to relaxation of the requirement that the regulatory sequence begin with the TSS. Thus, a TOP-like motif can be a sequence containing a stretch of at least five pyrimidines beginning within four nucleotides of the most frequent TSS, optionally interrupted by a single purine. See also, Philippe, et al., “La-related protein 1 (LARP1) repression of TOP mRNA translation is mediated through its cap-binding domain and controlled by an adjacent regulatory region”, Nucleic Acids Research, 46(3):1457-1469 (2018) doi: 10.1093/nar/gkx1237.

In some embodiments, the disclosed nucleic acids include a TOP or TOP-like motif. Typically, the TOP or TOP-like motif is characterized by one or more of the foregoing criteria, and begin within 10, 9, 8, 7, 6, 5, 4, 3, 2, 1, or 0 nucleotides from the TSS. In some embodiments, the TOP motif includes any four or more consecutive nucleic acids of the underlined portion of any of SEQ ID NOS:21-52 of Table 1, and/or any of SEQ ID NOS:21-52 or 87.

It is believed that the addition of a TOP or TOP-like sequence will increase the expression of an encoded polypeptide by several fold in cells with activated mTORC1 as compared to the expression level in cells with low levels of mTORC1 activity. The kidney epithelial cells that are affected by autosomal dominant polycystic kidney disease manifest very high levels of mTORC1 activity (Shillingford, et al., Proc Natl Acad Sci U SA., 103(14):5466-71 (2006). doi: 10.1073/pnas.0509694103, PMID: 16567633; PMCID: PMC1459378.). Thus, it is believed that expression of polypeptides encoded by the DNA and RNA constructs including a TOP or TOP-like control sequence would be substantially higher in affected cells than it would be in unaffected normal cells. Thus, the addition of the TOP sequence should limit any potential off target toxic effects of the proteins encoded by the constructs.

A number of diseases in addition to autosomal dominant polycystic kidney disease are characterized by elevated levels of mTOR activity. These include Tuberous Sclerosis, a large number of common tumors such as renal cell carcinoma and hepatocellular carcinoma, as well as genetic disorders associated with mutations in the genes encoding components of the mTOR signaling cascade (“mTOR-opathies”). The addition of the TOP motif to DNA or RNA constructs could help to target the expression of therapeutic polypeptides to affected cells in any disorder in which mTOR activity is inappropriately high in cells that manifest the disease phenotype. Thus, in some embodiments, the therapeutic polypeptide encoded by the nucleic acid is not a PC1-CTT polypeptide, but rather a different therapeutic polypeptide for the treatment of another disease such as Tuberous Sclerosis, cancer such as renal cell carcinoma and hepatocellular carcinoma, or another genetic disorder associate with a mutation in a gene encoding component of the mTOR signaling cascade. The therapeutic polypeptide can be selected by the practitioner based on the disease to be treated. For example, in genetic disorders, the therapeutic polypeptide may be a wildtype copy (or functional fragment or variant thereof) of the mutated, disease-causing polypeptide.

### 4. Host Cells

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell) can be used to, for example, produce the PC1-CTT polypeptides described herein.

### C. Delivery Vehicles

Any of the disclosed compositions including, but not limited to polypeptides and/or nucleic acids, can be delivered to target cells using a delivery vehicle. The delivery vehicles can be, for example, polymeric particles, inorganic particles, silica particles, liposomes, micelles, multilamellar vesicles, etc.

Delivery vehicles may be microparticles or nanoparticles. Nanoparticles are often utilized for intertissue application, penetration of cells, and certain routes of administration. The nanoparticles may have any desired size for the intended use. The nanoparticles may have any diameter from 10 nm up to about 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In some embodiments the nanoparticles can have a diameter less than 400 nm, less than 300 nm, or less than 200 nm. The range can be between 50 nm and 300 nm.

Thus, in some embodiments, the delivery vehicles are nanoscale compositions, for example, 10 nm up to, but not including, about 1 micron. However, it will be appreciated that in some embodiments, and for some uses, the particles can be smaller, or larger (e.g., microparticles, etc.). Although many of the compositions disclosed herein are referred to as nanoparticle or nanocarrier compositions, it will be appreciated that in some embodiments and for some uses the carrier can be somewhat larger than nanoparticles. Such compositions can be referred to as microparticulate compositions. For example, a nanocarriers according to the present disclosure may be a microparticle. Microparticles can a diameter between, for example, 0.1 and 100 μm in size.

### 1. Polymers

The delivery vehicle can be a particle containing one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), and copolymers thereof.

The delivery vehicle can contain one or more hydrophobic polymers. Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In some embodiments, the hydrophobic polymer is poly(lactic acid), poly(glycolic acid), or poly(lactic acid-co-glycolic acid).

The particle can contain one or more biodegradable polymers. Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water.

Biodegradable polymers in the particle can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxybutyrates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof.

The particles can contain one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In some embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end, or both.

In some embodiments, the particles contain a first amphiphilic polymer having a hydrophobic polymer block, a hydrophilic polymer block, and targeting moiety conjugated to the hydrophilic polymer block; and a second amphiphilic polymer having a hydrophobic polymer block and a hydrophilic polymer block but without the targeting moiety. The hydrophobic polymer block of the first amphiphilic polymer and the hydrophobic polymer block of the second amphiphilic polymer may be the same or different. Likewise, the hydrophilic polymer block of the first amphiphilic polymer and the hydrophilic polymer block of the second amphiphilic polymer may be the same or different.

In some embodiments the particle contains biodegradable polyesters or polyanhydrides such as poly(lactic acid), poly(glycolic acid), and poly(lactic-co-glycolic acid). The nanoparticles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(ε-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. Other polymers include PLGA-poly(F-carbobenzoxyl-L-lysine) (PLL) (i.e., PLGA-PLL).

The particles can also contain one or more polymer conjugates containing end-to-end linkages between the polymer and a targeting moiety or a detectable label. For example, a modified polymer can be a PLGA-PEG-peptide block polymer.

The particles can contain one or a mixture of two or more polymers. The particles may contain other entities such as stabilizers, surfactants, or lipids. The particles may contain a first polymer having a targeting moiety and a second polymer not having the targeting moiety. By adjusting the ratio of the targeted and non-targeted polymers, the density of the targeting moiety on the exterior of the particle can be adjusted.

The particles can contain an amphiphilic polymer having a hydrophobic end, a hydrophilic end, and a targeting moiety attached to the hydrophilic end. In some embodiments the amphiphilic macromolecule is a block copolymer having a hydrophobic polymer block, a hydrophilic polymer block covalently coupled to the hydrophobic polymer block, and a targeting moiety covalently coupled to the hydrophilic polymer block. For example, the amphiphilic polymer can have a conjugate having the structure A-B-X where A is a hydrophobic molecule or hydrophobic polymer, B is a hydrophilic molecule or hydrophilic polymer, and X is a targeting moiety. Exemplary amphiphilic polymers include those where A is a hydrophobic biodegradable polymer, B is PEG, and X is a targeting moiety that targets, binds.

In some embodiments the nanoparticle contains a first amphiphilic polymer having the structure A-B-X as described above and a second amphiphilic polymer having the structure A-B, where A and B in the second amphiphilic macromolecule are chosen independently from the A and B in the first amphiphilic macromolecule, although they may be the same.

### 2. Liposomes and Micelles

In some embodiments, the carrier is a liposome or micelle. Liposomes are spherical vesicles composed of concentric phospholipid bilayers separated by aqueous compartments. Liposomes can adhere to and form a molecular film on cellular surfaces. Structurally, liposomes are lipid vesicles composed of concentric phospholipid bilayers which enclose an aqueous interior (Gregoriadis, et al., Int. J. Pharm., 300, 125-30 2005; Gregoriadis and Ryman, Biochem. J., 124, 58P (1971)). Hydrophobic compounds associate with the lipid phase, while hydrophilic compounds associate with the aqueous phase.

Liposomes include, for example, small unilamellar vesicles (SUVs) formed by a single lipid bilayer, large unilamellar vesicles (LANs), which are vesicles with relatively large particles formed by a single lipid bilayer, and multi-lamellar vesicles (MLVs), which are formed by multiple membrane layers. Thus, the liposomes can have either one or several aqueous compartments delineated by either one (unilamellar) or several (multilamellar) phospholipid bilayers (Sapra, et al., Curr. Drug Deliv., 2, 369-81 (2005)). Multilamellar liposomes have more lipid bilayers for hydrophobic therapeutic agents to associate with. Thus, potentially greater amounts of therapeutic agent are available within the liposome to reach the target cell.

Liposomes have the ability to form a molecular film on cell and tissue surfaces. Clinical studies have proven the efficacy of liposomes as a topical healing agent (Dausch, et al., Kin Monatsbl Augenheilkd 223, 974-83 (2006); Lee, et al., Kin Monatsbl Augenheilkd 221, 825-36 (2004)). Liposomes have also been used in ophthalmology to ameliorate keratitis, corneal transplant rejection, uveitis, endophthalmitis, and proliferative vitreoretinopathy (Ebrahim, et al., 2005; Li, et al., 2007).

Liposomes have been widely studied as drug carriers for a variety of chemotherapeutic agents (approximately 25,000 scientific articles have been published on the subject) (Gregoriadis, N Engl J Med 295, 765-70 (1976); Gregoriadis, et al., Int. J. Pharm. 300, 125-30 (2005)). Water-soluble anticancer substances such as doxorubicin can be protected inside the aqueous compartment(s) of liposomes delimited by the phospholipid bilayer(s), whereas fat-soluble substances such as amphotericin and capsaicin can be integrated into the phospholipid bilayer (Aboul-Fadl, Curr Med Chem 12, 2193-214 (2005); Tyagi, et al., J Urol 171, 483-9 (2004)). Topical and vitreous delivery of cyclosporine was drastically improved with liposomes (Lallemand, et al., Eur J Pharm Biopharm 56, 307-18 2003). Delivery of chemotherapeutic agents lead to improved pharmacokinetics and reduced toxicity profile (Gregoriadis, Trends Biotechnol 13, 527-37 (1995); Gregoriadis and Allison, FEBS Lett 45, 71-4 1974; Sapra, et al., Curr Drug Deliv 2, 369-81 (2005)). More than ten liposomal and lipid-based formulations have been approved by regulatory authorities and many liposomal drugs are in preclinical development or in clinical trials (Barnes, Expert Opin Pharmacother 7, 607-15 (2006); Minko, et al., Anticancer Agents Med Chem 6, 537-52 (2006)). The safety data with respect to acute, subchronic, and chronic toxicity of liposomes has been assimilated from the vast clinical experience of using liposomes in the clinic for thousands of patients.

Carriers such as liposomes and micelles can be formed from one or more lipids, which can be neutral, anionic, or cationic at physiologic pH. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids. In some embodiments, the liposomes contain a phosphaditylcholine (PC) head group, and optionally sphingomyelin. In another embodiment, the liposomes contain DPPC. In a further embodiment, the liposomes contain a neutral lipid, such as 1,2-dioleoylphosphatidylcholine (DOPC).

In certain embodiments, the liposomes are generated from a single type of phospholipid. In some embodiments, the phospholipid has a phosphaditylcholine head group, and, can be, for example, sphingomyelin. The liposomes may include a sphingomyelin metabolite. Sphingomyelin metabolites used to formulate the liposomes include, without limitation, ceramide, sphingosine, or sphingosine 1-phosphate. The concentration of the sphingomyelin metabolites included in the lipids used to formulate the liposomes can range from about 0.1 mol % to about 10 mol %, or from about 2.0 mol % to about 5.0 mol %, or can be in a concentration of about 1.0 mol %.

Suitable cationic lipids in the liposomes include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids may be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine). The molar ratio of a first phospholipid, such as sphingomyelin, to second lipid can range from about 5:1 to about 1:1 or 3:1 to about 1:1, or from about 1.5:1 to about 1:1, or the molar ratio is about 1:1.

In some embodiments, liposomes or micelles include phospholipids, cholesterols and nitrogen-containing lipids. Examples include phospholipids, including natural phospholipids such as phosphatidylcholine, phosphatidylserine, phosphatidylglycerol, phosphatidylinositol, phosphatidylethanolamine, phosphatidic acid, cardiolipin, sphingomyelin, egg yolk lecithin, soybean lecithin, and lysolecithin, as well as hydrogenated products thereof obtained in a standard manner. It is also possible to use synthetic phospholipids such as dicetyl phosphate, distearoylphosphatidylcholine, dipalmitoylphosphatidylcholine, dipalmitoylphosphatidylethanolamine, dipalmitoylphosphatidylserine, eleostearoylphosphatidylcholine, eleostearoylphosphatidylethanolamine as well as homo-poly{N′—[N-(2-aminoethyl)-2-aminoethyl]aspartamide}P[Asp(DET)] and block-catiomer poly(ethyleneglycol) (PEG)-b-P[Asp(DET)].

In some embodiments, the liposomes are long circulating liposomes or stealth liposomes such as those reviewed in Immordino, et al, Int J Nanomedicine, 1(3):297-315 (2006)), which is specifically incorporated by reference herein in its entirety. For example, liposomes have been developed with surfaces modified with a variety of molecules including glycolipids and sialic acid. Long-circulating liposomes can include, for example, synthetic polymer poly-(ethylene glycol) (PEG) in liposome composition. The PEG on the surface of the liposomal carrier can extend blood-circulation time while reducing mononuclear phagocyte system uptake (stealth liposomes) and serve as an anchor for the targeting moiety.

Antibodies and antibody fragments are widely employed for targeting moieties for liposomes due to the high specificity for their target antigens. Referred to immunoliposomes, methods of generated targeted liposomes by coupling of antibodies to the liposomal surface are known in the art. Such techniques include, but are not limited to, conventional coupling and maleimide based techniques. See, for example, (Paszko and Senge, Curr Med Chem., 19(31):5239-77 (2012), Kelly, et al., Journal of Drug Delivery, Volume 2011 (2011), Article ID 727241, 11 pages).

The micelles can be polymer micelles, for example, those composed of amphiphilic di-or tri-block copolymers made of solvophilic and solvophobic blocks (see, e.g., Croy and Kwon, Curr Pharm Des., 12(36):4669-84 (2006)).

### III. Methods of Making

### A. Methods for Producing PC1-CTT Polypeptides

Isolated PC1-CTT polypeptides can be obtained by, for example, chemical synthesis or by recombinant production in a host cell. To recombinantly produce a PC1-CTT polypeptides, a nucleic acid containing a nucleotide sequence encoding the polypeptide, typically a vector such as those discussed above, can be used to transform, transduce, or transfect a bacterial or eukaryotic host cell (e.g., an insect, yeast, or mammalian cell). In general, nucleic acid constructs include a regulatory sequence operably linked to a nucleotide sequence encoding the PC1-CTT polypeptides. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well know in the art include, for example, Escherichia coli strains such as BL-21, and cultured mammalian cells such as CHO cells.

In eukaryotic host cells, a number of viral-based expression systems can be utilized to express PC1-CTT polypeptides. Viral based expression systems are well known in the art and include, but are not limited to, baculoviral, SV40, retroviral, or vaccinia based viral vectors.

Mammalian cell lines that stably express variant costimulatory polypeptides can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, PC1-CTT polypeptides can be produced by (a) ligating amplified sequences into a mammalian expression vector such as pcDNA3 (Invitrogen Life Technologies), and (b) transcribing and translating in vitro using wheat germ extract or rabbit reticulocyte lysate.

PC1-CTT polypeptides can be isolated using, for example, chromatographic methods such as DEAE ion exchange, gel filtration, and hydroxylapatite chromatography. For example, PC1-CTT polypeptides in a cell culture supernatant or a cytoplasmic extract can be isolated using a protein G column. As discussed above, in some embodiments, PC1-CTT polypeptides can be “engineered” to contain an amino acid sequence that allows the polypeptides to be captured onto an affinity matrix. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify the polypeptides.

Methods for introducing random mutations to produce variant polypeptides are known in the art. Random peptide display libraries can be used to screen for PC1-CTT polypeptides that interact with NNT. Techniques for creating and screening such peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially.

### 1. Fusion Proteins

When the polypeptide, e.g., PC1-CTT polypeptide, includes a heterologous sequence or sequences it is most typically prepared as a fusion protein. Fusion proteins or chimeric proteins are proteins created through the joining of two or more nucleic acid sequences that originally coded for separate polypeptides. Translation of this fusion gene results in a single polypeptide with functional properties derived from each of the original polypeptides. Recombinant fusion proteins are created artificially by recombinant DNA technology. e.g., as discussed above.

### 2. Polypeptide Conjugates

Additionally or alternatively, PC1-CTT polypeptides can be prepared as protein conjugates with one or more functional elements (e.g., protein transduction domains, fusogenic peptides, targeting molecules, tags, etc. chemically conjugated thereto. Methods for attaching peptides, small molecules, and other compounds to polypeptides are well known in the art and can include use of bifunctional chemical linkers such as N-succinimidyl (4-iodoacetyl)-aminobenzoate; sulfosuccinimidyl(4-iodoacetyl)-aminobenzoate; 4-succinimidyl-oxycarbonyl-V-(2-pyridyldithio) toluene; sulfosuccinimidyl-6-[alpha-methyl-V-(pyridyldithiol)-toluami-do]hexanoate; N-succinimidyl-3-(-2-pyridyldithio)-proprionate; succinimidyl-6-[3 (-(-2-pyridyldithio)-proprionamido] hexanoate; sulfosuccinimidyl-6-[3 (-(-2-pyridyldithio)-propionamido] hexanoate; 3-(2-pyridyldithio)-propionyl hydrazide, Ellman's reagent, dichlorotriazinic acid, S-(2-thiopyridyl)-L-cysteine, and the like. Further bifunctional linking molecules are discussed in, for example, U.S. Pat. Nos. 5,349,066, 5,618,528, 4,569,789, 4,952,394, and 5,137,877.

The linker can cleavable or noncleavable. Highly stable linkers can reduce the amount of payload that falls off in circulation, thus improving the safety profile, and ensuring that more of the payload arrives at the target cell. Linkers can be based on chemical motifs including disulfides, hydrazones or peptides (cleavable), or thioethers (noncleavable) and control the distribution and delivery of the active agent to the target cell. Cleavable and noncleavable types of linkers have been proven to be safe in preclinical and clinical trials (see, e.g., Brentuximab vedotin which includes an enzyme-sensitive linker cleavable by cathepsin; and Trastuzumab emtansine, which includes a stable, non-cleavable linker). In particular embodiments, the linker is a peptide linker cleavable by Edman degradation (Bgchor, et al., Molecular diversity, 17 (3): 605-11 (2013)).

### B. Methods for Producing Isolated Nucleic Acid Molecules

Isolated nucleic acid molecules encoding polypeptides such as PC1-CTT polypeptides and other therapeutic proteins can be produced by standard techniques, including, without limitation, common molecular cloning and chemical nucleic acid synthesis techniques. For example, polymerase chain reaction (PCR) techniques can be used to obtain an isolated nucleic acid encoding a variant costimulatory polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoroamidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also be obtained by mutagenesis. Polypeptide encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of amino acid positions that can be modified include those described herein.

### C. Methods of Making Delivery Vehicles

### 1. Particle Formation

Methods of making delivery vehicles are known in the art. See, e.g., U.S. Published Application No. 2019/0330317, which is specifically incorporated by reference herein in its entirety. For example, in some embodiments, a particle is prepared using an emulsion solvent evaporation method. For example, a polymeric material is dissolved in a water immiscible organic solvent and mixed with a drug solution or a combination of drug solutions. In some embodiments the polymer solution contains one or more polymer conjugates as described above.

In another embodiment, a multimodal nanoparticle is prepared using nanoprecipitation methods or microfluidic devices. A polymeric material is mixed with a drug or drug combinations in a water miscible organic solvent. Methods of making nanoparticles using microfluidics are known in the art. Suitable methods include those described in U.S. Published Application No. 2010/0022680 A1 by Karnik et al.

Other methods of making particles include, but are not limited to, solvent evaporation, hot melt microencapsulation, solvent removal, spray-drying, and hydrogel particles formation.

Methods of manufacturing liposomes are known in the art and can include, for example, drying down of the lipids from organic solvents, dispersion of the lipids in aqueous media, purification of the resultant liposomes, and analysis of the final product. Some methods of liposome manufacture include, for example, extrusion methods, the Mozafari method, the polyol dilution method, the bubble method, and the heating method.

The micelles may be prepared in a conventional manner, for example, by reversed-phase evaporation, ether injection, surfactant-based techniques, etc. Polymer micelle formulations utilizing a block copolymer having a hydrophilic segment and a hydrophobic segment have been disclosed, e.g., in U.S. Application No. 2016/0114058, WO 2009/142326 A1 and WO 2010/013836 A1.

### 2. Methods of Encapsulating or Attaching Molecules to Particles

Delivery vehicles can be used to deliver the disclosed compositions. For example, a PC1-CTT polypeptide or other therapeutic protein, with or without a heterologous sequence, or a nucleic acid encoding the same can be encapsulated in the delivery vehicle. Additionally or alternatively, the PC1-CTT polypeptide and/or nucleic acid can be conjugated to one or more elements of the delivery vehicle.

In addition or alternative to any of the foregoing, the delivery vehicle can include one or more functional elements, such as protein transduction domains, fusogenic peptides, targeting molecules, etc., can be encapsulated or more preferably conjugated, most preferably exterior surface conjugated, to the delivery vehicle. These can be coupled using standard techniques. The targeting molecule or therapeutic molecule to be delivered can be coupled directly to the polymer or to a material such as a fatty acid which is incorporated into the polymer.

Functionality refers to conjugation of a ligand to the surface of the particle via a functional chemical group (carboxylic acids, aldehydes, amines, sulfhydryls and hydroxyls) present on the surface of the particle and present on the ligand to be attached. Functionality may be introduced into the particles in two ways.

The first is during the preparation of the particles, for example during the emulsion preparation of particles by incorporation of stabilizers with functional chemical groups.

A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This second class also includes a process whereby amphiphilic molecules such as fatty acids, lipids or functional stabilizers may be passively adsorbed and adhered to the particle surface, thereby introducing functional end groups for tethering to ligands.

Methods of polymer synthesis are described, for instance, in Braun et al. (2005) Polymer Synthesis: Theory and Practice. New York, NY: Springer. The polymers may be synthesized via step-growth polymerization, chain-growth polymerization, or plasma polymerization.

In some embodiments an amphiphilic polymer is synthesized starting from a hydrophobic polymer terminated with a first reactive coupling group and a hydrophilic polymer terminated with a second reactive coupling group capable of reacting with the first reactive coupling group to form a covalent bond. One of either the first reactive coupling group or the second reactive coupling group can be a primary amine, where the other reactive coupling group can be an amine-reactive linking group such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. One of either the first reactive coupling group or the second reactive coupling group can be an aldehyde, where the other reactive coupling group can be an aldehyde reactive linking group such as hydrazides, alkoxyamines, and primary amines. One of either the first reactive coupling group or the second reactive coupling group can be a thiol, where the other reactive coupling group can be a sulfhydryl reactive group such as maleimides, haloacetyls, and pyridyl disulfides.

In some embodiments a hydrophobic polymer terminated with an amine or an amine-reactive linking group is coupled to a hydrophilic polymer terminated with complimentary reactive linking group. For example, an NHS ester activated PLGA can be formed by reacting PLGA-CO(OH) with NHS and a coupling reagent such as dicyclohexylcarbodiimide (DCC) or ethyl(dimethylaminopropyl) carbodiimide (EDC). The NHS ester activated PLGA can be reacted with a hydrophilic polymer terminated with a primary amine, such as a PEG-NH₂ to form an amphiphilic PLGA-b-PEG block copolymer.

In some embodiments a conjugate of an amphiphilic polymer with a functional moiety is formed using the same or similar coupling reactions. In some embodiments the conjugate is made starting from a hydrophilic polymer terminated on one end with a first reactive coupling group and terminated on a second end with a protective group. The hydrophilic polymer is reacted with a targeting moiety having a reactive group that is complimentary to the first reactive group to form a covalent bond between the hydrophilic polymer and the targeting moiety. The protective group can then be removed to provide a second reactive coupling group, for example to allow coupling of a hydrophobic polymer block to the conjugate of the hydrophilic polymer with the targeting moiety. A hydrophobic polymer terminated with a reactive coupling group complimentary to the second reactive coupling group can then be covalently coupled to form the conjugate. Of course, the steps could also be performed in reverse order, i.e., a conjugate of a hydrophobic polymer and a hydrophilic polymer could be formed first followed by deprotection and coupling of the targeting moiety to the hydrophilic polymer block.

In some embodiments a conjugate is formed having a moiety conjugated to both ends of the amphiphilic polymer. For example, an amphiphilic polymer having a hydrophobic polymer block and a hydrophilic polymer block may have targeting moiety conjugated to the hydrophilic polymer block and an additional moiety conjugated to the hydrophobic polymer block. In some embodiments the additional moiety can be a detectable label. In some embodiments the additional moiety is a therapeutic, prophylactic, or diagnostic agent. For example, the additional moiety could be a moiety used for radiotherapy. The conjugate can be prepared starting from a hydrophobic polymer having on one end a first reactive coupling group and another end first protective group and a hydrophilic polymer having on one end a second reactive coupling group and on another end a second protective group. The hydrophobic polymer can be reacted with the additional moiety having a reactive coupling group complimentary to the first reactive coupling group, thereby forming a conjugate of the hydrophobic polymer to the additional moiety. The hydrophilic polymer can be reacted with a targeting moiety having a reactive coupling group complimentary to the second reactive coupling group, thereby forming a conjugate of the hydrophilic polymer to the targeting moiety. The first protective group and the second protective group can be removed to yield a pair of complimentary reactive coupling groups that can be reacted to covalently link the hydrophobic polymer block to the hydrophilic polymer block.

### IV. Pharmaceutical Compositions

The disclosed compositions alone or in a delivery vehicle can be formulated with appropriate pharmaceutically acceptable carriers into pharmaceutical compositions for administration to an individual in need thereof. The formulations can be administered enterally (e.g., oral) or parenterally (e.g., by injection or infusion).

The disclosed compositions can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesional, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

In some embodiments, the disclosed compositions are administered systemically by, for example, injection or infusion. In some embodiments, the compositions are administered locally by injection or infusion. For example, in more specific embodiments, the compositions are administered to the kidney or a tumor (e.g., by injection or infusion), or to the central nervous system, particularly the brain, by convection enhanced delivery (CED). In a particular embodiment, the compositions are delivered to the kidney by retrograde ureteral administration. Such an approach has shown promise for limiting immune reactions when the target tissue is the kidney. See, e.g., Chung et al., Nephron Extra, 1:217-223 (2011), doi.org/10.1159/000333071. In yet another embodiment, direct intrarenal injection is utilized (Kuemmerle, et al., Pediatr Nephrol., 14(2):152-7 (2000). doi: 10.1007/s004670050033. PMID: 10684368.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required nanocarrier size in the case of dispersion and/or by the use of surfactants. In many cases, isotonic agents, for example, sugars or sodium chloride are included

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, exemplary methods of preparation include vacuum-drying and freeze-drying techniques which yield a powder of the active ingredient plus any additional desired ingredient from a previously sterile-filtered solution thereof. The powders can be prepared in such a manner that the nanocarriers are porous in nature, which can increase dissolution of the nanocarriers. Methods for making porous nanocarriers are well known in the art.

Enteral formulations are prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Exemplary hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins.

Carrier also includes all components of the coating composition, which may include plasticizers, pigments, colorants, stabilizing agents, and glidants. Formulations can be prepared using one or more pharmaceutically acceptable excipients, including diluents, preservatives, binders, lubricants, disintegrators, swelling agents, fillers, stabilizers, and combinations thereof.

Controlled release dosage formulations can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

The disclosed compounds alone or in a particle formulation can also be applied topically. Topical administration can include application to the lungs, nasal, oral (sublingual, buccal), vaginal, or rectal mucosa. In some embodiments, the compositions are administered in combination with transdermal or mucosal transport elements.

A wide range of mechanical devices designed for pulmonary delivery of therapeutic products can be used, including but not limited to, nebulizers, metered dose inhalers, and powder inhalers, all of which are familiar to those skilled in the art. Some specific examples of commercially available devices are the Ultravent® nebulizer (Mallinckrodt Inc., St. Louis, Mo.); the Acorn® II nebulizer (Marquest Medical Products, Englewood, Colo.); the Ventolin® metered dose inhaler (Glaxo Inc., Research Triangle Park, N.C.); and the Spinhaler® powder inhaler (Fisons Corp., Bedford, Mass.). Nektar, Alkermes and Mannkind all have inhalable insulin powder preparations approved or in clinical trials where the technology could be applied to the formulations described herein.

Oral formulations may be in the form of chewing gum, gel strips, tablets, capsules, or lozenges. Oral formulations may include excipients or other modifications to the particle which can confer enteric protection or enhanced delivery through the GI tract, including the intestinal epithelia and mucosa (see Samstein, et al., Biomaterials, 29(6):703-8 (2008).

Transdermal formulations may also be prepared. These will typically be ointments, lotions, sprays, or patches, all of which can be prepared using standard technology. Transdermal formulations can include penetration enhancers.

### V. Methods of Treatment

### A. Methods of Treating ADPKD

Methods of treating Autosomal Dominant Polycystic Kidney Disease are provided. Approximately 85% of ADPKD cases, are Type 1 ADPKD cases caused by mutations in PKD1, which encodes polycystin-1 (PC1). The disclosed compositions can be administered to a subject in need thereof in an effective amount to reduce, limit, prevent, and/or reverse one or more symptoms of ADPKD, particularly symptoms and complications of Type 1 disease.

In some cases, a subject having, or at risk of developing, ADPKD can be administered a composition including one or more of the disclosed compositions to treat the subject. For example, in some cases, a subject (e.g., a human) having, or at risk of developing, ADPKD can be administered a PC1-CTT polypeptide, or a nucleic acid encoding the same, in an effective amount to reduce or eliminate one or more symptoms of ADPKD. Examples of symptoms and complications associated with ADPKD include, without limitation, back pain, side pain, headache, a feeling of fullness (e.g., in the abdomen), increased size of the abdomen (e.g., due to an enlarged kidney), blood in the urine, high blood pressure, loss of kidney function (e.g., kidney failure), heart valve abnormalities (e.g., mitral valve prolapse), colon problems (e.g., diverticulosis), development of an aneurysm (e.g., a brain aneurysm), and ED. For example, one or more of the disclosed compositions can be administered to a subject in need thereof as described herein to reduce the severity of one or more symptoms and/or complications associated with ADPKD by, for example, 10, 20, 30, 40, 50, 60, 70, 80, 90, 95, or more percent.

In some cases, a subject having, or at risk of developing, ADPKD can be administered a PC1-CTT polypeptide, or a nucleic acid encoding the same, to reduce or eliminate one or more cysts (e.g., one or more renal cysts) within the subject. For example, a PC1-CTT polypeptide, or a nucleic acid encoding the same, can be administered to a subject in need thereof as described herein to reduce the size (e.g., volume) of a cyst within the subject by, for example, 10, 20, 30, 40, 50, 60, 70, 80, 90, 95, or more percent. In some embodiments, the cystic index (also referred to as a cystic burden, e.g., the percentage of an organ such as a kidney that is occupied by cysts) is reduced in the subject by, for example, 10, 20, 30, 40, 50, 60, 70, 80, 90, 95, or more percent. Any appropriate method can be used to determine the size of a cyst (e.g., a renal cyst) and/or a cystic index within a subject (e.g., a subject having, or at risk of developing, ADPKD). For example, ultrasound, computed tomography (CT) scanning, magnetic resonance imaging (MRI), and/or histological images can be used to determine the size of a cyst (e.g., a renal cyst) and/or a cystic index of a subject. In some cases, a cystic index can be determined as described elsewhere (see, e.g., Irazabal et al., J. Vis. Exp., (100):e52757 (2015); and Hopp et al., J. Clin. Invest., 122(11):4257-42-73 (2012)).

In some cases, a subject having, or at risk of developing, ADPKD can be administered an effective amount of a PC1-CTT polypeptide, or a nucleic acid encoding the same, to reduce the weight and/or volume of one or both kidneys within the subject and/or to reduce the body weight of the subject. In some embodiments, the weight and/or volume of a kidney within the subject and/or the body weight of the subject is reduced by, for example, 10, 20, 30, 40, 50, 60, 70, 80, 90, 95, or more percent. Any appropriate method can be used to determine the weight and/volume. Imaging such as ultrasound, computed tomography (CT) scanning, magnetic resonance imaging (MM) can be used to determine the volume of a kidney.

In some embodiments, the compositions are administered in an effective amount to preserve (e.g., maintain) or increase the vasculature (e.g., a capillary count) within the subject. In some embodiments, vasculature is increased by, for example, 10, 20, 30, 40, 50, 60, 70, 80, 90, 95, or more percent. Any appropriate method can be used to detect the vasculature (e.g., the capillaries) within a subject.

Examples of subjects having, or at risk of developing, ADPKD that can be treated as described herein include mammals, including, but not limited to, humans, non-human primates (e.g., monkeys), dogs, cats, horses, cows, pigs, sheep, mice, and rats. In some cases, methods for treating a subject (e.g., a human) having, or at risk of developing, ADPKD, also can include identifying a subject as having, or as being at risk of developing, ADPKD. Any appropriate method can be used to identify a subject as having, or as being at risk of developing, ADPKD. For example, imaging techniques (e.g., ultrasound, CT scan, and MRI) and/or laboratory tests (e.g., genetic testing for mutation of one or both copies of the polycystic kidney disease gene 1 (PKD1) can be used to identify a subject as having, or as being at risk of developing, ADPKD.

Effective amounts can vary depending on the route of administration, the age of the subject, the sex of the subject, the general health condition of the subject, excipient usage, the possibility of co-usage with other therapeutic treatments such as use of other agents, and the judgment of the treating physician. An effective amount of a composition containing a PC1-CTT polypeptide, or a nucleic acid encoding the same, can be any amount that can treat a subject having, or at risk of developing, ADPKD as described herein preferably without producing significant toxicity to the subject. The effective amount can remain constant or can be adjusted as a sliding scale or variable dose depending on the subject's response to treatment. Various factors can influence the actual effective amount used for a particular application. For example, the frequency of administration, duration of treatment, use of multiple treatment agents, route of administration, and/or severity of the ADPKD in the subject being treated may require an increase or decrease in the actual effective amount administered.

The composition can be administered in any appropriate frequency or duration. The frequency and/or duration of administration can be any frequency and/or duration that can treat a subject having, or at risk of developing, ADPKD without producing significant toxicity to the subject. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency. The effective duration can vary from several weeks to several months, from several months to several years, or from several years to a lifetime. In some cases, the effective duration can range in duration from about 10 years to about a lifetime. Multiple factors can influence the actual effective duration used for a particular treatment. For example, an effective duration can vary with the frequency of administration, effective amount, use of multiple treatment agents, and/or route of administration. In a particular embodiment, particularly where the method includes a gene therapy approach, e.g., the composition is nucleic acid encoding the therapeutic polypeptide, and optionally is deployed by a vector (e.g., a lentiviral vector), a single administration may be sufficient to treat or cure the disease.

Each of the different PC1-CTT compositions disclosed herein can be administered alone or in any combination with one or more additional active agents and/or interventions. In all cases, the combination of agents can be part of the same admixture or administered as separate compositions. In some embodiments, the separate compositions are administered through the same route of administration. In other embodiments, the separate compositions are administered through different routes of administration. Examples of additional active agents and other interventions that can be used as described herein to treat one or more symptoms of ADPKD and/or one or more complications associated with ADPKD include, without limitation, an inhibitor of a vasopressin receptor (e.g., tolvaptan or lixivaptan), angiotensin-converting enzyme (ACE) inhibitors, angiotensin II receptor blockers (ARBs), pain relievers (e.g., acetaminophen), antibiotics, restricted diet (e.g., a diet low in methionine, high in choline, and/or high in betaine content), maintaining a healthy body weight, exercising regularly, undergoing dialysis, undergoing a kidney transplant, and others described include, e.g., in the background section, and otherwise known in the art.

Some methods further include monitoring the severity of disease and/or treatment response. Imaging can be used to monitor phenotypes (e.g., cysts, kidney size/volume, etc.), and/or any appropriate urine and/or plasma biomarker can be used to monitor the treatment response of ADPKD in a subject. In some cases, one or more metabolic or cellular pathways can be assessed using any appropriate urine and/or plasma biomarker and can be assessed at different time points. For example, urine and/or plasma biomarkers (e.g., MCP-1, FGF23, IGF-1, TGFB1, homocysteine, glutathione, or combinations thereof) can be used to assess a treatment response (e.g., biomarker changes).

As introduced above, the compositions can be administered locally, regionally, or systemically using a variety of methods of administration. In a particular embodiment, compositions are administered to the kidney by a retrograde ureteral approach.

B. Methods for Treating Diseases with Increased mTOR

As introduced above, TOP and TOP-like sequence motifs, if properly designed and situated in the sequence of the DNA or RNA construct, can render the translation and stability of the mRNA susceptible to regulation by the activity of mTOR Complex 1 (mTORC1), thus the mTORC1 signaling pathway. Thus, nucleic acids such as DNA and RNA constructs, including, but not limited to mRNAs and DNA vectors, can include a TOP or TOP-like sequence to increase the expression of an encoded therapeutic polypeptide in cells with activated mTORC1 as compared to the expression level in cells with low levels of mTORC1 activity.

As introduced above, the kidney epithelial cells that are affected by autosomal dominant polycystic kidney disease manifest very high levels of mTORC1 activity (Shillingford, et al., Proc Nat Acad Sci USA., 103(14):5466-71 (2006). doi: 10.1073/pnas.0509694103, PMID: 16567633; PMCID: PMC1459378.). It is believed that expression of the PC1-CTT polypeptides encoded by the delivered DNA or RNA constructs including a TOP or TOP-like motif would be substantially higher in affected cells than it would be in unaffected normal cells. Thus, the addition of the TOP sequence should limit any potential off target toxic effects of the polypeptides encoded by the constructs.

In other embodiments, the same or similar strategy is used to treat a number of other diseases also characterized by elevated levels of mTOR activity. The addition of a TOP or TOP-like motif to DNA or RNA constructs can help localize expression of a desired therapeutic proteins in affected cells in any disorder in which mTOR activity is inappropriately high in cells that manifest the disease phenotype. These include a large number of common tumors such as renal cell carcinoma and hepatocellular carcinoma, arthritis, insulin resistance, osteoporosis, as well as genetic disorders associated with mutations in the genes encoding components of the mTOR signaling cascade (“mTOR-opathies”) including tuberous sclerosis complex (TSC), focal cortical dysplasia type II (FCDII), hemimegaloencephaly (HME), polyhydramnios, megalocephaly, and symptomatic epilepsy (PMSE) syndrome, among others. See, e.g., Nguyen and Bordey, “Convergent and Divergent Mechanisms of Epileptogenesis in mTORopathies,” Front. Neuroanat., Vol. 15, Article 664695 (2021) doi.org/10.3389/fnana.2021.664695, and Zou, et al., mTOR signaling pathway and mTOR inhibitors in cancer: progress and challenges,” Cell & Bioscience, 10(31) (2020) doi.org/10.1186/s13578-020-00396-1, each of which is specifically incorporated by reference herein in its entirety. The therapeutic polypeptide is selected by the practitioner based on the disease to be treated.

Thus, methods of treating a subject in need thereof by administering to the subject an effective amount of a nucleic acid construct with a TOP or TOP-like motif operably linked to, or otherwise incorporated into, a sequence encoding a therapeutic polypeptide are provided. Typically, the subject has a disease or disorder including dysregulated cells characterized by elevated levels of mTOR activity (e.g., mTORC1 expression and/or bioactivity). Preferably, the compositions improve one or more symptoms of the disease or disorder. Any of these nucleic acid compositions can utilize the features discussed in more detail above, particularly with respect to nucleic acid compositions. Thus, for example, the compositions can be deployed as RNA (e.g., mRNA or RNA vector), or DNA (e.g., DNA expression construct or vector), can include functional moieties including, but not limited to, PTD, fusogenic peptides, targeting moieties, etc. conjugated thereto, or to a delivery vehicle for delivery thereof.

In some embodiments, the disease is a genetic disorder including one or more disease causing mutations, and the therapeutic polypeptide is a wildtype copy or other fragment or variant thereof that restores the function or bioactivity lost by the mutated gene/protein. For example, genetic mutations leading to mTORC1 hyperactivity were first identified in the prototypical mTORopathy, TSC. Individuals with TSC have germline and somatic inactivating mutations in the TSC1 or TSC2 genes, leading to mTORC1 activation in multiple organs, including the brain. More recently, the emergence of advanced sequencing techniques led to the seminal discovery that FCDII and HME are caused by de novo brain somatic mutations in mTORC1 pathway genes that occur during neurodevelopment, and it has been demonstrated that brain somatic mutations in PI3K, AKT3, and mTOR are causal of HME and brain somatic mutations in mTOR cause FCDII. Brain somatic mutations in TSC1 or TSC2, in the absence of germline mutations, also lead to FCDII. PMSE is caused by a homozygous deletion in the STE20-related kinase adaptoru (Strada) gene, a modulator of mTORC1 signaling via the AMP-activated protein kinase (AMPK)-TSC1/2 pathway. Pathogenic variants in at least 14 distinct genes along the PI3K-mTOR pathway and GATOR1 complex, including PI3K, PTEN, AKT3, TSC1, TSC2, RHEB, MTOR, STRADA, DEPDC5, NPRL2, NPRL3, KPTN, SZT2, and TBC1D7, have been linked to malformation of cortical development (MCD) and epilepsy. Mutations in some genes (i.e., MTOR) occur much more frequently than in others (i.e., KPTN, SZT2, and TBC1D7). Thus, in some embodiments, a subject having a mutation in one of the foregoing genes is administered an effective amount of a nucleic acid construct with a TOP or TOP-like motif operably linked to, or otherwise incorporated into, a sequence encoding a wildtype copy of the mutated gene.

In some embodiments, the disease is a cancer including cancer cells characterized by abnormally high levels of mTORC1 activity. The types of cancer that can be treated with the provided compositions and methods include, but are not limited to, cancers such as vascular cancer such as multiple myeloma, as well as adenocarcinomas and sarcomas. The cancer can be, for example, bone, bladder, brain, breast, cervical, colo-rectal, esophageal, kidney, liver, lung, nasopharyngeal, pancreatic, prostate, skin, stomach, or uterine cancer. In some embodiments, the disclosed compositions are used to treat multiple cancer types concurrently. The compositions can also be used to treat metastases or tumors at multiple locations.

Examples of peptides that can be used to treat cancers and that can be utilized in the disclosed compositions and methods are known in the art and include, but are not limited, to antimicrobial peptides, peptides that target transduction pathways, peptides that target the cell cycle, peptides that induce cell death, peptides that target transcription factors, and peptides that counter aspects of mTORC1 activation. See, e.g., Table 2, and Marqus, et al., “Evaluation of the use of therapeutic peptides for cancer treatment,” J Biomed Sci 24, 21 (2017). doi.org/10.1186/s12929-017-0328-x, which is specifically incorporated by reference herein in its entirety.

TABLE 2
Therapeutic peptides and their uses (adapted from Marqus, et al., supra).

| | |
|---|---|
| Peptide name | Cell lines examined |

Antimicrobial peptides

| | |
|---|---|
| Magainin II | Bladder cancer cells: RT4 pathologic grade 1, 647 V grade |
| | 2, and 486P grade 4 |
| NRC-3 and NRC-7 | Breast cancer: MDA-MB-231, MDA-MB-468, SKBR3, |
| | MCF-7 and paclitaxel resistant MCF-7 (MCF-7-TX400) |
| | and murine mammary 4 T1 carcinoma cells |
| Buforin IIb | Cervical carcinoma (HeLa), leukemia (Jurkat cells) |
| | and lung cancer (NCI-H460) cells |
| BR2 | Cervical carcinoma (HeLa), colon cancer (HCT116) |
| | and murine melanoma (B16-F10) cells |

Therapeutic peptides target transduction pathway

| | |
|---|---|
| PNC-2 and PNC-7 | Pancreatic cancer (MIA-PaCa) cells |
| Cardiac | Pancreatic cancer (HPAC), renal carcinoma (SW156), |
| natriuretic peptides | breast adenocarcinoma (HCCI428), ovarian |
| | adenocarcinoma (NIHOVCAR-3), modularly thyroid |
| | carcinoma (TT), glioblastoma (LNZTA3WT4) and lung |
| | carcinoma (NCI-H1963) cells |
| RGD-PEG-Suc-PD0325901 | Glioblastoma (U87MG) cells |
| VWCS | Head and neck squamous cell carcinoma (HNSCC) and oral |
| | epidermoid carcinoma (KB) cells |
| FWCS | Head and neck squamous cell carcinoma (HNSCC) and oral |
| | epidermoid carcinoma (KB) cells |

Therapeutic peptides target cell cycle

| | |
|---|---|
| p16 | Pancreatic cancer (AsPC-1 and BxPC-3) cells |
| Bac-7-ELP-p21 | Ovarian carcinoma (SKOV-3) cells |
| Pen-ELP-p21 | Cervical carcinoma (HeLa) and ovarian carcinoma |
| | (SKOV-3) cells |

Therapeutic peptides induce cell death

| | |
|---|---|
| TAT-Bim | Murine T-cell lymphoma (EL4), pancreatic cancer |
| | (Panc-02) and melanoma (B16-F10) cells |
| Poropeptide-Bax | Melanoma (SK-MEL-28) cells |
| R8-Bax | Cervical carcinoma (HeLa) and marine mammary |
| | carcinoma (TS/A) cells |
| CT20p-NP | Breast cancer (MCF-7 or MDA-MB-231) and colon |
| | cancer (HCT-116) cells |
| RRM-MV | Squamous cell carcinoma (COLO16) and malignant |
| | melanoma (MM96L), and murine melanoma (B16-F10) |
| | cells |
| RRM-IL12 | Mouse melanoma (B16-F10) cells |

Therapeutic peptides target tumor suppressor protein

| | |
|---|---|
| PNC-27 | Cervical carcinoma (HeLa), colon cancer (SW1417 and |
| | H11299), breast cancer (MDA-MB-453 and MCF-7), |
| | osteosarcoma (SAOS2), leukemia (K562), pancreatic cancer |
| | (MIA-PaCa-2) and melanoma (A-2058) cells. Rat k-ras- |
| | transformed pancreatic cancer (TUC-3) and transformed |
| | endothelial (E49) cells |
| PNC-21 | Cervical carcinoma (HeLa), colon cancer (SW1417 and |
| | H1299), breast cancer (MDA-MB-453), and osteosarcoma |
| | (SAOS2) cells. Rat k-ras-transformed pancreatic cancer |
| | (TUC-3) and transformed endothelial (E49) cells |
| PNC-28 | Breast cancer (MDA-MB-453), colon cancer (H1299 and |
| | SW1417), osteosarcoma (SAOS2), cervical carcinoma |
| | (HeLa) and pancreatic cancer (MiaPaCa-2) cells. Rat k-ras- |
| | transformed pancreatic cancer (TUC-3) and transformed |
| | endothelial (E49) cells |
| Tat-αHDM2 | Melanoma (MM-23, MM-24 and MM-26), retinoblastoma |
| | (Y79 and WERI), osteosarcoma (U2OS), and cervical |
| | carcinoma (C33A) cells |

Therapeutic peptides target transcription factors

| | |
|---|---|
| Int-H1-S6A, F8A | Breast cancer (MCF-7) cells |
| Pen-ELP-H1 | Breast cancer (MCF-7) cells |
| BACl-ELP-H1 | Glioma (U-87 MG and D54) and marine glioma (C6) cells |

The invention can be further understood by the following numbered paragraphs:
1. A polypeptide including SEQ ID NO:1 or a functional fragment or variant thereof and a heterologous sequence, optionally packaged in or otherwise associated with a delivery vehicle.
2. The polypeptide of paragraph 1 including the delivery vehicle.
3. A polypeptide including SEQ ID NO:1 or a functional fragment or variant thereof packaged in or otherwise associated with a delivery vehicle, optionally wherein the polypeptide includes a heterologous sequence.
4. The polypeptide of paragraph 3 including the heterologous sequence.
5. A variant polypeptide including at least 70% and less than 100% sequence identity to SEQ ID NO:1 or functional fragment thereof, optionally wherein the polypeptide includes a heterologous sequence, is packaged in or otherwise associated with a delivery vehicle, or a combination thereof.
6. The polypeptide of paragraph 5, including the heterologous sequence.
7. The polypeptide of paragraphs 5 or 6, including the delivery vehicle.
8. The polypeptide of any one of paragraphs 1-7, wherein the heterologous sequence includes one or more of a protein transduction domain, fusogenic polypeptide, targeting signal, expression and/or purification tag.
9. The polypeptide of any one of paragraphs 1-8, wherein the variant includes at least 75% sequence identity of SEQ ID NO:1, or a functional fragment thereof.
10. The polypeptide of any one of paragraphs 1-9, wherein the variant or fragment is between 25 and 200 amino acids inclusive, or any subrange or specific integer therebetween.
11. The polypeptide of any one of paragraphs 1-10, wherein the polypeptide can interact with nicotinamide nucleotide transhydrogenase (NNT), optionally wherein interaction includes the ability to co-immunoprecipitate.
12. The polypeptide of any one of paragraphs 1-8, including a mutated PEST motif with reduce activity.
13. The polypeptide of any one of paragraphs 1-12 including a mitochondrial localization signal.
14. The polypeptide of paragraph 13, wherein the mitochondrial localization signal includes the amino acid sequence of SEQ ID NOS:98 or 99, or a variant thereof with a least 70% sequence identity thereto.
15. The polypeptide of any one of paragraphs 1-14 including a heterologous mitochondrial localization signal.
16. The polypeptide of paragraph 15, wherein the amino acid sequence of SEQ ID NO:98 and/or SEQ ID NO:99 is absent.
17. The polypeptide of paragraph 16, including the amino acid sequence of SEQ ID NO:100 or a fragment or variant thereof with at least 70% sequence identity thereto.
18. The polypeptide of paragraph 16 including a variant of the amino acid sequence of SEQ ID NO:1 wherein the amino acid sequence of SEQ ID NOS:98 or 99 is deleted, and the heterologous mitochondrial localization signal is inserted in its place or appended to the N- or C-terminus of the polypeptide.
19. A nucleic acid including a nucleic acid encoding the polypeptide of any one of paragraphs 1-18, optionally packaged in a delivery vehicle.
20. The nucleic acid of paragraph 19 including or encoding a TOP or TOP-like motif.
21. A nucleic acid including a nucleic acid encoding a therapeutic polypeptide operably linked to a TOP or TOP-like motif or its encoding sequence, optionally packaged in a delivery vehicle.
22. The nucleic acid of paragraph 21, wherein the therapeutic polypeptide includes SEQ ID NO:1 or a functional fragment or variant thereof.
23. The nucleic acid of any one of paragraphs 19-22, wherein the TOP or TOP-like motif includes at least 4 pyrimidines beginning within four nucleotides of the transcriptional start site, optionally beginning at the transcription start site.
24. The nucleic acid of paragraphs 22 or 23, wherein the TOP or TOP-like motif includes the nucleic acid sequence of the underlined portion of any of SEQ ID NOS:21-52 of Table 1, and/or any of SEQ ID NOS:21-52 or 87.
25. The nucleic acid of any one of paragraphs 19-24, wherein the nucleic acid is RNA or DNA.
26. The nucleic acid of any one of paragraphs 19-25, wherein the nucleic acid includes an expression control sequence(s).
27. The nucleic acid of any one of paragraphs 19-26, wherein the nucleic acid is a vector.
28. The nucleic acid of paragraph 27, wherein the nucleic acid is a viral vector.
29. The nucleic acid of any one of paragraphs 19-28, wherein the nucleic acid is mRNA.
30. The nucleic acid of any one of paragraphs 19-29, wherein the nucleic acid includes a promotor.
31. The nucleic acid of paragraph 30, wherein the promotor is a kidney-specific promoter.
32. The nucleic acid of any one of paragraphs 19-31 including one or more of a protein transduction domain, fusogenic polypeptide, or targeting signal conjugated thereto.
33. The nucleic acid of any one of paragraphs 19-32 including the delivery vehicle.
34. The polypeptide of any one of paragraphs 1-18 or nucleic acid of any one of paragraphs 19-34, wherein the delivery vehicle is formed of polymeric particles, inorganic particles, silica particles, liposomes, micelles, or multilamellar vesicles, optionally wherein the delivery vehicles include one or more of a protein transduction domain, fusogenic polypeptide, or targeting signal conjugated thereto.
35. A pharmaceutical composition including the any one of paragraphs 1-18 or nucleic acid of any one of paragraphs 19-34 alone or packaged in a delivery vehicle optionally formed from formed of polymeric particles, inorganic particles, silica particles, liposomes, micelles, or multilamellar vesicles, optionally wherein the delivery vehicles include one or more of a protein transduction domain, fusogenic polypeptide, or targeting signal conjugated thereto.
36. A method of treating a subject in need thereof including administering the subject an effective amount of the pharmaceutical composition of paragraph 35.
37. The method of paragraph 36, wherein the subject has a genetic disorder.
38. The method of paragraph 37, wherein the genetic disorder is Autosomal Dominant Polycystic Kidney Disease (ADPKD).
39. The method of paragraph 38, wherein the composition is administered by a retrograde ureteral approach.
40. A method of treating Autosomal Dominant Polycystic Kidney Disease (ADPKD) including administering a subject with ADPKD the pharmaceutical composition of paragraph 35.
41. A method of treating a subject with a disease characterized by increase mTOR activity including administering the subject a pharmaceutical composition including the nucleic acid of any one of paragraphs 21-34.
42. The method of paragraph 41, wherein the disease is selected from ADPKD, arthritis, insulin resistance, osteoporosis, cancer, and mTOR-opathies optionally selected from tuberous sclerosis complex (TSC), focal cortical dysplasia type II (FCDII), hemimegaloencephaly (HME), polyhydramnios, megalocephaly, and symptomatic epilepsy (PMSE) syndrome.
43. The method of paragraph 41, wherein the disease is a genetic disorder, and the therapeutic polypeptide is a wildtype copy or other fragment or variant thereof that restores the function or bioactivity lost by the mutated gene/protein of the genetic disorder.
44. The method of paragraph 41, wherein the disease is a cancer.
45. The method of paragraph 44, wherein the therapeutic polypeptide is cytotoxic to cancer cells, an antimicrobial peptide, a peptide that targets a transduction pathway, a peptide that targets the cell cycle, a peptide that induces cell death, a peptide that targets a transcription factor, and/or a peptide that counters an aspect of mTORC1 activation.
46. The method of paragraphs 44 or 45, wherein the therapeutic polypeptide is selected from the peptides of Table 2.

### EXAMPLES

### Example 1: PC1-CTT Suppresses ADPKD Phenotype in a Mitochondrial Enzyme-Dependent Manner

### Materials and Methods

### Mouse Models

All animal experiments were approved and conducted in accordance with Yale Animal Resources Center and Institutional Animal Care and Use Committee (JACUC) regulations (protocol #2019-20088). The ^{Pkd1fl/fl};Pax8^{rtTA};TetO-Cre (Ma, M., et al. Nature Genetics Vol. 45, pages 1004-12 (2013)) and Pkd1^{F/H}-BAC (Fedeles, S. V. et al. Nature Genetics Vol. 43, pages 639-47 (2011)). mouse models were used. Pkd1^{fl/fl};Pax8^{rtTA};TetO-Cre mice were generated on two distinct backgrounds by breeding in either C57BL/6J (stock no:000664, Jackson Laboratories) or C57BL/6N (stock no:005304, Jackson Laboratories) strains. Additionally, the 2HA-PC1-CTT; Pkd1^{fl/fl};Pax8^{rtTA};TetO-Cre were generated on both C57BL/6J and C57BL/6N backgrounds. Animals were maintained on a 12-hour light, 12 hour dark cycle, with 30-70% humidity and a temperature of 20-26° C. In each experiment, animals were age-matched and sex distribution in sexually mature mice was similar across groups (specific descriptions in Brief Description of the Drawings). Cre-negative littermates served as healthy wild-type (WT) controls.

Generation of the 2HA-PC1-CTT;Pkd1^{fl/fl};Pax8^{rtTA};TetO-Cre Mouse Model.

A 2HA-PKD1-CTT BAC construct, which encodes a protein corresponding to a 2XHA tag linked to the N-terminus of the final 600 bp of human PC1, was generated with published BAC recombineering technologies^{25,46,47}. The cDNA sequence encoding 2HA-PKD1-CTT was introduced into the pRosa26-DEST vector (catalog #21189, Addgene) such that a lox-Neo(R)-3×STOP-lox is followed by the sequence encoding 2HA-PKD1-CTT. A recombination cassette was constructed by flanking a rpsL+− kana selection cassette (catalog #20871, Addgene) with two same homology arms (1000 bp each arm) from pRosa26-DEST. Mouse Rosa26 BAC DNA was electroporated into DY380 bacteria that stably integrated a defective k prophage containing the red recombination genes exo, bet, and gam under a strong pL promoter controlled by the temperature sensitive cI857 repressor. The rpsL+-kana cassette was introduced into Rosa26 intron 1 region of the BAC after activation of the red recombination system at 42° C. under positive selection by kanamycin resistance. The rpsL+-kana cassette in this intermediate was replaced by introducing lox-Neo(R)-3×STOP-lox and 2×HA-PKD1-CTT fragment with Rosa26 homology arms under negative selection with streptomycin sensitivity conferred by the rpsL+gene after the activation of the red recombination system at 42° C. The final 2×HA-PKD1-CTT Rosa26 BAC was shown to contain only the intended recombination and no other rearrangement using DNA restriction fingerprinting, direct sequencing, and in vitro recombination in SW106 bacterial strain carrying an L-arabinose-inducible Cre gene.

Linearized modified BAC DNA purified by Contour-clamped homogeneous electric field (CHEF) electrophoresis was used for pronuclear injection to generate transgenic founder lines. The BAC transgenic lines were produced in (C57BL/6J×SJL/J) F₂ zygotes. Founders were identified by PCR genotyping, verified by sequencing of PCR products and BAC copy number was determined by genomic quantitative PCR (Fedeles, S. V. et al. Nature Genetics Vol. 43, pages 639-47 (2011); Cai, Y. et al. J Clin Invest Vol. 124, pages 5129-44 (2014)) (Dong, et al., Nat Genet (2021)). Two BAC founders with BAC copy numbers 2 or 4 were used in this study. All strains were backcrossed at least four generations with C57BL6 and are therefore thought to be at least 90% C57BL6 congenic. These animals were then crossed with Pkd1^{fl/fl}/Pax8^{rtTA};TetO-Cre mice to generate 2HA-PC1-CTT; Pkd1^{fl/fl};Pax8^{rtTA};TetO-Cre, on both C57BL/6J and C57BL/6N backgrounds.

### Generation of the 2HA-PC1-CTT;Pkd1^{fl/fl};Pkhd1-Cre Mouse Model

2HA-PC1-CTT;Pkd1^{fl/fl};Pax8^{rtTA};TetO-Cre mice, generated on the C57BL/6N background, were crossed with Pkd1^{fl/+};Pkhd1-Cre mice, generated on the C57BL/6J background. The Pkd1^{fl/fl};Pkhd1-Cre F₁ progeny that did or did not express the 2HA-PC1-CTT BAC transgene were analyzed comparatively at p14 (post-partum day 14). Additionally, 2-HA-PC1-CTT;Pkd1^{fl/+};Pkhd1-Cre mice littermates were also generated and evaluated at p14 to assess whether any phenotypic differences were present in non-cystic mice that expressed the 2HA-PC1-CTT BAC transgene.

### Cell Lines

HEK293 cells were cultured in DMEM supplemented with 10% fetal bovine serum (FBS), 1% penicillin/streptomycin, and 1% 1-glutamine at 37° C. These cells were then subjected to transient transfection following the protocol described in the transient transfection section described below.

### Human Specimens

Human kidney tissue from both ADPKD patients and non-affected controls were obtained from the Baltimore Polycystic Kidney Disease Research and Clinical Core Center (P30DK090868). Samples were surgically harvested according to the guidelines established by the Institutional Review Board of the University of Maryland and were then de-identified. Immunoblotting with anti-NNT (catalog #459170, Invitrogen) and anti-actin (catalog #A2228, Sigma Aldrich) antibodies was performed using the protocol described in the Western blot section below.

### Mouse Kidney Tissue Harvest

Mice were euthanized according to established IACUC protocols. Tail or toe tissue from mice was acquired when animals were under anesthesia for genotype confirmation. Retro-orbital blood was also collected from these anesthetized mice. The left kidney was excised, weighed, snap-frozen in liquified N₂ and stored at −80° C. for biochemistry analysis. The right kidney was excised, weighed, and fixed in 4% paraformaldehyde. Fixed kidneys were then sectioned in half along their sagittal axes, infiltrated with 30% sucrose overnight and embedded in OCT for further imaging.

### Serum Creatinine and BUN Measurement

Retro-orbital blood was collected from anesthetized mice prior to sacrifice and centrifuged in Plasma Separator Tubes with Lithium Heparin (BD) to separate plasma. Serum creatinine and Blood Urea Nitrogen (BUN) analysis were then performed.

### Immunoprecipitation

Immunoprecipitation from Crude Mitochondria Fractions Prepared from Pkd1F/H-BAC mice

Kidneys from Pkd1^{F/H}-BAC and WT controls were isolated and homogenized with a Potter-Elvehjem® homogenizer, followed by a series of differential centrifugations at 4° C. according to the following protocol (Wieckowski, et al., Nat Protoc Vol. 4, 1582-90 (2009)).: lysates were initially submitted to low-speed centrifugation at 740 g-force/RCF (740 g) for 5 minutes, and the collected supernatant was once again submitted to low-speed centrifugation at 740 g for 5 minutes. The collected supernatant was submitted to high-speed centrifugation at 9,000 g for 10 minutes. The supernatant was discarded, and the pellet resuspended and submitted to high-speed centrifugation at 10,000 g for 10 minutes. This final step was repeated twice, and the pellet containing crude mitochondria (both mitochondria and mitochondria-associated ER membranes (MAMs)) was resuspended in PBS. The samples were incubated with 3 mM 3,3′-Dithiobis(sulfosuccinimidylpropionate) (DTSSP) (catalog #803200-50 mg, Sigma Aldrich) to covalently cross link interacting proteins at room temperature (RT) for 30 minutes in a rocking shaker and then the crosslinking reaction was quenched with 20 mM Tris-HCl pH 7.4. Samples were thereafter submitted to a high-speed 10,000 g centrifugation for 10 minutes and resuspended in 100 μl of PBS+1% SDS, followed by a 30-minutes immunoprecipitation with 25 μl of anti-HA magnetic beads (catalog #88837, ThermoFischer Scientific) and 4 washes with TENT buffer (10 mM Tris-HCl, 0.1M NaCl, 1 mM EDTA, 5% v/v Triton X-100@). The final immunoprecipitate was either snap frozen in liquified N₂ and stored at −80° C. for further proteomic analysis or eluted in 40 μl of 2× Laemmli sample buffer (catalog #1610747, Bio-Rad) with 300 mM DTT at 95° C. for 10 minutes for immunoblotting.

Immunoprecipitation from Kidney Lysates Prepared from 2HA-PC1-CTT;Pkd1^{fl/fl};Pax8^{rtTA};TetO-Cre Mice

Kidneys harvested from 2HA-PC1-CTT;Pkd1^{fl/fl};Pax8^{rtTA};TetO-Cre mice in both “N” and “J” backgrounds and from Pkd1^{fl/fl};Pax8^{rtTA};TetO-Cre mice in the “N” background were snap frozen and stored at −80° C. Homogenization was performed on ice using a motorized tissue grinder (catalog #1214136, Fisher Scientific) in Tris lysis buffer (50 mM Tris pH 7.4, 100 mM NaCl, 0.5% NP-40, 0.5% Triton X-100@, 2 mM EDTA) supplemented with complete mini EDTA-free protease inhibitor cocktail tablets (catalog #11836170001, Roche) and PhosSTOP phosphatase inhibitor cocktail tablets (catalog #04906837001, Roche). Homogenates were then sonicated for 30 seconds (2×15 second bursts at 40% power) and incubated for 45 minutes on ice to complete protein solubilization. Lysates were centrifuged at 8000 rpm for 15 minutes. Protein concentrations were measured with the Protein Assay Dye Reagent Concentrate (catalog #5000006, Bio-Rad). Anti-HA magnetic beads (catalog #88837, ThermoFischer Scientific) were equilibrated in lysis buffer (50 μL beads per reaction in 500 μl of lysis buffer) for 10 minutes at room temperature on a rocking shaker, and then incubated with a total of 4 mg of tissue lysate per sample, overnight, at 4° C. Following four 5-minute washes with 1 ml lysis buffer, the dynabeads were magnetically recovered and precipitated proteins were eluted in 60 μl of 2× Laemmli sample buffer (catalog #1610747, Bio-Rad) with 300 mM Dithiothreitol (DTT). One-third of the eluted proteins (20 μl) was loaded per sample per gel for immunoblotting.

### Proteomic Analysis

Proteomic analysis on material immunoprecipitated from the Pkd1^{F/H}-BAC kidneys was performed according to standard operating procedures. Immunoprecipitated proteins were subjected to chloroform: methanol: water protein extraction, after which they were reduced, alkylated and trypsin digested for follow up LC-MS/MS bottom-up data collection. Samples were analyzed with an Orbitrap Fusion mass spectrometer and Mascot Search Engine software was utilized for protein identifications.

### Western Blotting

Snap-frozen mouse kidneys were homogenized as described in the 2HA-PC1-CTT;Pkd1^{fl/fl};Pax8^{rtTA};TetO-Cre whole kidney lysate immunoprecipitation. Protein concentrations were measured with the Protein Assay Dye Reagent Concentrate (catalog #5000006, Bio-Rad). 20-40 μg of protein from whole kidney lysate or 20 μl of IP eluted proteins were separated on 4-20% Mini-PROTEAN TGX Precast Protein Gels (catalog #4561093, Bio-Rad) and electrophoretically transferred to a nitrocellulose membrane. Loading only surpassed the 20-40 μg range in the immunoblot in which loading of 60 μg of whole kidney lysate was necessary to identify PC1-CTT in both 2HA-PC1-CTT;Pkd1^{fl/fl};Pax8^{rtTA};TetO-Cre and Pkd1F/H-BAC mice. For western-blotting of human renal tissue, homogenization was performed using a Polytron mechanical homogenizer in Tris lysis buffer (50 mM Tris pH 7.4, 100 mM NaCl, 0.5% NP-40, 0.5% Triton X-100@, 1 mM EDTA) for 15 seconds, at 300 rpm, on ice. Homogenates were then sonicated for 1 minute, with 3 single continuous 15-second bursts at 40% power separated by a 5-second pause, left on ice for 60 minutes to complete protein solubilization, and centrifuged for 10 minutes at 10,000 g. Membranes were sequentially incubated with blocking buffer (PBS, 6% (w/v) powdered milk/BSA, 0.1% Tween) followed by overnight incubation with primary antibodies.

The primary antibodies used in this study were: anti-NNT (Catalog #459170, Invitrogen; Catalog #sc-390215, Santa Cruz), anti-NNT-HRP (Catalog #sc-390236HRP, Santa Cruz), anti-PC1-C-terminus (Catalog #EJH002, Kerafast), anti-HA-Peroxidase (Catalog #12013819001, Roche), Anti-HA-680 (Catalog #26183-D680, ThermoFischer Scientific), anti-actin (Catalog #A2228, Sigma Aldrich), anti-TOMM20 (Catalog #NBP1-81556, Novus Biologicals), anti-Total OXPHOS Cocktail (Catalog #MS604-300, Abcam) and anti-VDAC-HRP (Catalog #sc-390996HRP, Santa Cruz). All primary antibodies were used at a 1:1000 dilution, except for conjugated primaries anti-NNT-HRP (1:500), anti-HA-Peroxidase (1:500), anti-HA-680 (1:500) and anti-VDAC-HRP (1:250). Unconjugated primary antibodies were detected using species-specific infrared (IR)-conjugated secondary IgG (1:5,000; Catalog #926-32211 and #926-68070, Li-Cor). Mitochondrial extract from rat heart tissue lysate (Catalog #ab110341, Abcam) was utilized as positive control. Membranes were visualized with either the Odyssey Infrared Imager (Li-Cor Biosciences) or Odyssey Fc (Li-Cor Biosciences) for chemiluminescence detection. Individual bands were quantified using ImageJ software (https://imagej.nih.gov/ij/, NIH).

### Transient Transfection in Cultured Cells

Lipofectamine 2000 (Catalog #11668019, ThermoFischer Scientific) to transiently transfect HEK293 cells following the manufacturer's protocol. Cells were transfected with the 2HA-PC1-CTT construct¹⁷. Briefly, the sequence encoding the final 200 amino acid of human PC1(4102-4302) with an N-terminal 2×HA tag was cloned into the cDNA3.1 zeo vector. The 2HA-PC1-CTT sequence is identical to that expressed in Pkd1-KO+CTT mice.

### Immunofluorescence Staining in Cells

HEK293 cells grown on poly-L coated coverslips were fixed with 4% PFA in PBS for 30 minutes at room temperature followed by a 15-minute treatment with permeabilization buffer (PBS, 1 mM MgCl₂, 0.1 mM CaCl₂, 0.1% BSA, 0.3% Triton X-100®). Cells were blocked with goat serum dilution buffer (GSDB; 16% filtered goat serum, 0.3% Triton X-100®, 20 mM NaPi, pH 7.4, 150 mM NaCl) for 30 minutes, followed by a one-hour incubation period with primary antibodies (1:100) diluted in GSDB. The primary antibodies utilized were anti-PC1-C-terminus (catalog #EJH002, Kerafast), anti-NNT (catalog #459170, Invitrogen) and anti-TOMM20 (catalog #NBP1-81556, Novus Biologicals). Following three PBS washes, samples were incubated with secondary antibodies (1:200) diluted in GSDB for one hour and then washed again with PBS. Alexa Fluor conjugated antibodies (Alexa-594, 647; Catalog #A11032 and #A31573 respectively, Life Technologies Invitrogen) were used as secondary reagents. Finally, coverslips were mounted on slides with VectaShield mounting medium (catalog #H-1000-10, Vector Laboratories) and imaged using a Zeiss LSM780 confocal microscope.

Images are the product of 8-fold line averaging and contrast and brightness settings were chosen so that all pixels were in the linear range. This experiment was repeated three times. Mander's colocalization analysis was performed using Fiji 3-ImageJ (National Institutes of Health, Bethesda, MD) and Coloc 2-ImageJ plug-in. Briefly, the region of interest (ROI), defined as the non-nuclear area of a single transfected cell, was determined by tracing individual cells and subtracting all staining in Hoechst-positive areas. Staining for PC1-C-terminus and NNT were analyzed exclusively within the ROI and the overlap was assessed through Mander's colocalization analysis. Mander's colocalization analysis between TOMM20 and NNT, also in single cells, was used as a positive experimental control.

### Mouse Tissue Immunohistochemistry

Kidneys were fixed and processed for immunohistochemistry as described in the “Mouse Kidney Tissue Harvest” section above. Four-μm thick sections were heated in 10-mM citrate buffer for 15 minutes. Slides were then blocked with 0.5% H₂0₂ in methanol for 30 minutes, followed by three 5-minute 0.01M PBS washes and further blocking with skim milk in PBS for 1 hour at room temperature. Overnight incubation was performed with anti-NNT antibody (catalog #sc-390215, Santa Cruz) at a 1:50 dilution followed by detection using VectaStain Elite ABC-HRPkit (catalog #PK-6200, Vector Laboratories) according to the manufacturer's instructions.

### Proliferation Assay

Kidneys were fixed and processed for immunofluorescence as described in the “Mouse Kidney Tissue Harvest” section above. Antigen retrieval for ki67 was performed on 4-μm thick sections by heating slides in a 10-mM citrate buffer for 20 minutes. After a 30-minute incubation period with blocking buffer (PBS, 1% BSA, 10% goat serum), sections were co-incubated with anti-ki67 (catalog #VP-RM04, Vector Laboratories) and anti-Na,K-ATPase α subunit (catalog #a5, DSHB) primary antibodies at a 1:100 dilution followed by detection with Alexa Fluor-conjugated secondary antibodies (Life Technologies Invitrogen) at a 1:200 dilution and Hoechst nuclear staining (catalog #H3570, Molecular Probes Invitrogen).

Confocal images were obtained using a Zeiss LSM780 confocal microscope. Images are the product of 8-fold line averaging and contrast and brightness settings were chosen so that all pixels were in the linear range. Anti-Na, K-ATPase α-subunit was used as a tubular marker. Three images were acquired in the upper, middle, and lower third of the kidney by a blinded investigator who also quantified the percentage of ki67 positive nuclei relative to total tubular nuclei in these nine independent images. A total of at least 2000 tubular nuclei were counted per animal.

### Morphological Analyses

Whole kidney images from hematoxylin and eosin-stained sagittal kidney sections were obtained at a 4× magnification using automated image acquisition by the scan slide module in MetaMorph (Molecular Devices). The whole kidney was defined as the region of interest and the ImageJ default auto threshold function was employed to measure cystic and tubular area relative to total kidney area by an individual blinded to experimental conditions.

### NNT Enzymatic Assay

Levels of NNT enzymatic activity were measured in N-Pkd1-KO, N-Pkd1-KO+CTT and N-WT mice (Shimomura, et al., Methods Enzymol Vol. 457, 451-80 (2009)). Briefly, 10-week-old pre-cystic (Ma, M., et al. Nature Genetics Vol. 45, pages 1004-12 (2013)) mice were euthanized according to established protocols. Left kidneys were extracted and partitioned in half along their coronal axis. One half of the kidney (approximately 70 mg) was used for mitochondrial preparation, which was performed using the Q Proteome™ Mitochondria Isolation Kit (catalog #37612, Qiagen). The Qiagen protocol was followed in detail until step 11a but the final wash (step 12a) was not performed in order to follow the suggested NNT-assay protocol (Shimomura, et al., Methods Enzymol Vol. 457, 451-80 (2009)).

Suspension of the final pellet was carried out with 20 μl of mitochondria storage buffer (catalog #37612, Qiagen), a volume sufficient to allow the NNT assay and protein determination. Protein concentrations were measured with the Protein Assay Dye Reagent Concentrate (catalog #5000006, Bio-Rad), revealing a mean concentration of 7.64+/−2.2 mg/ml. These values were within the protocols' 5-10 mg/ml predicted concentration. The assay medium was prepared and used within 24 hours and was composed of 50 mM Tris-HCl (pH8.0), 0.5% Brij 35, 1 mg/ml of lysolecithin and 300 μM of both NADPH and APAD. The assay was performed with 1 ml of assay buffer and 10 μl of the mitochondrial suspension and was read with a Bechman DU-640 UV-Vis spectrophotometer with a time-course setting: one measurement per second at a 375 nm wavelength, the chosen wavelength for reduced APAD. WT C57BL/6J and C57BL/6N were used as negative and positive controls, respectively. The former confirmed assay specificity (revealing no relevant activity) while the latter confirmed sustained linear activity for several minutes, as reported in the original protocol³⁸. This experiment was repeated three times. The protocol predicts occasional delays in activity initiation, reflecting the time taken for mitochondria to become permeable to substrates, and therefore an investigator blinded to genotype marked the starting point of linear slope. Reactions were measured for a minimum of 150 seconds of optimal linear slope. The enzymatic activity was calculated by dividing the variation in optical density (OD; y axis) per variation in time (x axis, in seconds). Results are presented in activity per mg of protein, as established by the protocol (Shimomura, et al., Methods Enzymol Vol. 457, 451-80 (2009)).

### Genomic DNA Isolation and Quantitative RT-PCR

The DNeasy Blood & Tissue kit (catalog #69504, Qiagen) was used to extract genomic DNA from all 2HA-PC1-CTT;Pkd1^{fl/fl};Pax8^{rtTA};TetO-Cre and Pkd1^{fl/fl};Pax8^{rtTA};TetO-Cre mice included in the 16-week cohort, starting with 20 mg of kidney tissue from each animal and following the manufacturer's instructions. Of note, the optional 2-minute treatment with 4 μl of RNAse A (100 mg/ml) was performed at room temperature to obtain RNA-free genomic DNA in transcriptionally active tissues. Quantitative RT-PCR (qRT-PCR) was performed using iTaq Universal SYBR Green Supermix (catalog #172-5121, Bio-Rad). All samples were loaded in triplicates and reactions and data acquisition were performed using the Agilent real-time PCR system with its associated software. GAPDH levels were measured to normalize gene expression. Primers are listed below:

Determination of Pax8rtTA and TetO-Cre copy numbers
(SEQ ID NO: 90)
Pax8rtTA F: 5′-AAGTCATAAACGGCGCTCTG-3′
(SEQ ID NO: 91)
Pax8rtTA R: 5′-CAGTACAGGGTAGGCTGCTC-3′
(SEQ ID NO: 92)
TetO-Cre F: 5′-TCCATAGAAGACACTGGGACC-3′
(SEQ ID NO: 93)
TetO-Cre R: 5′-AGTAAAGTGTACAGGATCGGC-3′
(SEQ ID NO: 94)
GAPDH F: 5′-TGGTGTGACAGTGACTTGGG-3′
(SEQ ID NO: 95)
GAPDH R: 5′-GTCCTCAGTGTAGCCCAAGA-3′

Mouse samples were normalized to DNA obtained from a control mouse that expressed a single copy of both Pax8^{rtTA} and TetO-Cre. All animals included in the present cohort presented a 1:1 or 2:1 ratio for both genes when compared to controls, confirming homozygosity or heterozygosity for both Pax8^{rtTA} and TetO-Cre alleles.

### Determination of Pkd1 Rearrangement Levels

Both forward and reverse primers are situated within the floxed region (exons 2-4) of the Pkd1 gene in the Pkd1^{fl/fl};Pax8^{rtTA};TetO-Cre. The primer sequences used were:

(SEQ ID NO: 96)
Pkd1-Forward: 5′-TCTGTCATCTTGCCCTGTTCC-3′
(SEQ ID NO: 97)
Pkd1-Reverse: 5′-GTTGCACTCAAATGGGTTCCC-3′.

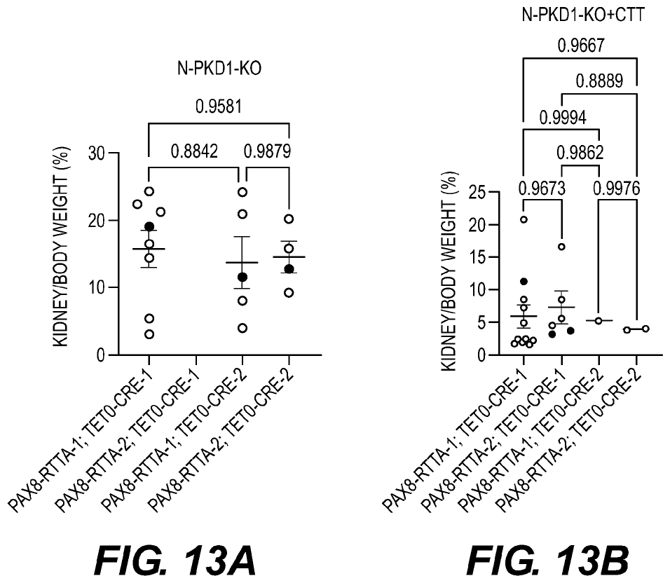

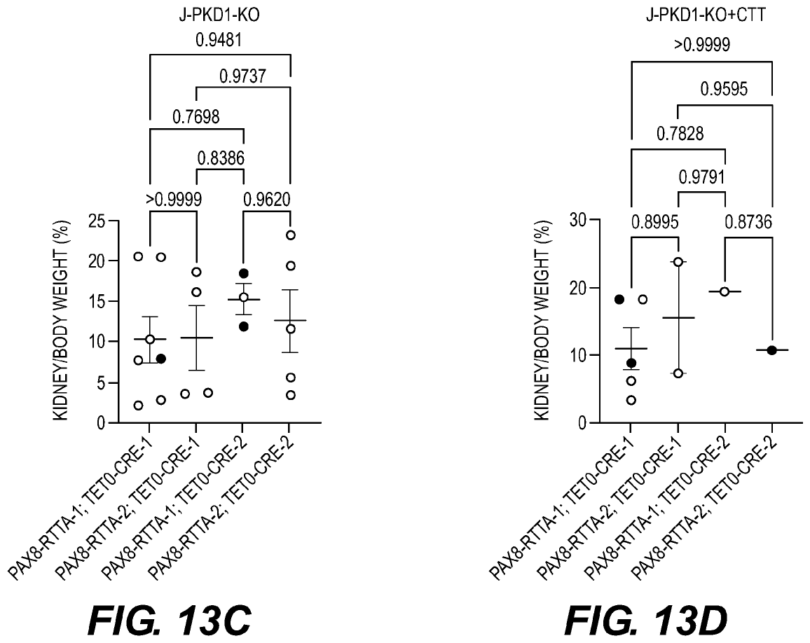

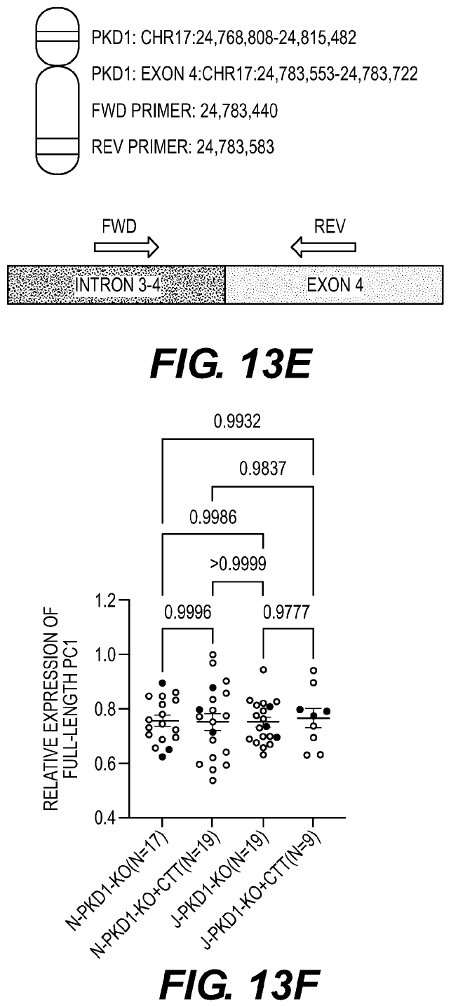

The reverse primer is located in Chr17:24,783,583 (exon 4) and the forward primer is contained in the prior intron at position Chr17:24,783,440. The amplified segment is therefore only present in the presence of intact WT Pkd1. GAPDH levels were measured to normalize gene expression. All cystic mice were then normalized to the same four healthy controls and, exhibited lower expression of WT Pkd1 compared to these WT controls. The ratio established between each cystic animal and WT controls served to define the rearrangement levels shown in FIG. 13F.

### Metabolomics

LC/MS-based analyses were performed on a Q Exactive Plus benchtop orbitrap mass spectrometer equipped with an Ion Max source and a HESI II probe, which was coupled to a Vanquish UHPLC (ThermoFisher Scientific). Polar metabolite extraction and detection methods were adapted from previous literature with minor modifications to be compatible with NAD(P)(H) measurement (Lu, et al., Antioxid Redox Signal Vol. 28, 167-179 (2018), Shen, et al., Cell Vol. 171, 771-782 e11 (2017), Wang, et al., Cell Metab Vol. 30, 539-555 e11 (2019)). (bioRxiv2021.09.22.461361). Specifically, 40 mg of snap-frozen tissue samples were ground using a mortar and pestle on dry ice. Metabolites were extracted with 800 μl 4/4/2 acetonitrile/methanol/water with 0.1 M formic acid, vortexed and incubated on dry ice for 3 minutes, and neutralized with 69.6 μl 15% ammonium bicarbonate. The samples were incubated on dry ice for 20 minutes, then centrifuged at 21,000 g for 20 minutes at 4° C., and 150 μl of supernatant were transferred to an LC-MS glass vial for analysis. Polar metabolites were analyzed on Xbrige BEH Amide XP HILIC Column, 100A, 2.5 μm, 2.1 mm×100 mm (catalog #186006091, Waters) for chromatographic separation. The column oven temperature was 27° C., the injection volume 10 μl and the autosampler temperature 4° C. Mobile phase A was 5% acetonitrile, 20 mM ammonium acetate/ammonium hydroxide, pH 9, and mobile phase B was 100% acetonitrile. LC gradient conditions at flow rate of 0.220 ml/min were as follows: 0 minutes: 85% B, 0.5 minutes: 85% B, 9 minutes: 35% B, 11 minutes: 2% B, 13.5 minutes: 85% B, 20 minutes: 85% B. The mass data were acquired in the polarity switching mode with full scan in a range of 70-1000 m/z, with the resolution at 70,000, with the AGC target at 1e⁶, the maximum injection time at 80 ms, the sheath gas flow at 50 units, the auxiliary gas flow at 10 units, the sweep gas flow at 2 units, the spray voltage at 2.5 kV, the capillary temperature at 310° C., and the auxiliary gas heater temperature at 370° C. Compound Discoverer (ThermoFisher Scientific) was used to pick peaks and integrate intensity from raw data. The metabolite lists were filtered with minimal peak area>1e⁷ and annotated by searching against an in-house chemical standard library with 5-ppm mass accuracy and 0.5 min retention time windows followed by manual curation. The data were normalized to tissue protein content. The PCA plots were generated by ClustVis (biit.cs.ut.ee/clustvis/) and the volcano plots using R script (r-project.org/). NAD(P)(H) levels in the kidney extracts were analyzed on SeQuant ZIC-pHILIC polymeric 5 μm, 150×2.1 mm column (EMD-Millipore, 150460). Mobile phase A: 20 mM ammonium carbonate in water, pH 9.6 (adjusted with ammonium hydroxide), and mobile phase B: acetonitrile. The column was held at 27° C., injection volume 5 μl, and an autosampler temperature of 4° C. LC conditions at flow rate of 0.15 ml/min as following: 0 minutes: 80% B, 0.5 minutes: 80% B, 20.5 minutes: 20% B, 21.3 minutes: 20% B, 21.5 minutes: 80% B till 29 minutes. The data were analyzed using the Xcalibur software.

### Quantification and Statistical Analysis

Data quantification and plotting were performed using GraphPad Prism software (graphpad.com/scientific-software/prism/), with the exception of metabolomic data, which were analyzed and plotted with ClustVis and R as described in the “Metabolomics” section above. Sample sizes for experiments involving Pkd1^{fl/fl};Pax8^{rtTA};TetO-Cre mice were chosen based on strategies used in previous analyses that have examined similar questions with the same experimental animal system (Ma, M., et al. Nature Genetics Vol. 45, pages 1004-12 (2013) (Dong, et al., Nat Genet (2021)). Power calculations were performed prospectively for the Pkd1^{fl/fl};Pkhd1-Cre (+/−CTT) cohort, based on the CTT-dependent phenotype suppression previously observed in the Pkd1^{fl/fl};Pax8^{rtTA};TetO-Cre model and on the observed variation in kidney-to-body weight ratios at p14 in cystic animals without CTT expression, which indicated that 12 animals per group would give 80% power to detect a 35% change in kidney-to-bodyweight ratio at a significance threshold of P<0.05. Data were expressed as means±SEM. Student's t-test or Mann-Whitney U test was used for pairwise comparisons, as indicated in the Brief Description of the Drawings.

One-way analysis of variance (ANOVA) followed by Tukey's multiple-comparison test was used for multiple comparisons. P<0.05 was considered statistically significant. The only mice excluded from the present study were those below the 3rd percentile of body weight derived from the Pkd1^{fl/fl};Pkhd1-Cre cohort sacrificed at p14 to ensure that naturally occurring developmentally delayed runt pups (Burkholder, et al., Curr Protoc Mouse Biol Vol. 2, 145-165 (2012)). would not bias the present analyses. There is no correlation between the occurrence of runts and the Pkd1^{fl/fl};Pkhd1-Cre genotype (Ma, M., et al. Nature Genetics Vol. 45, pages 1004-12 (2013); (Fedeles, S. V. et al. Nature Genetics Vol. 43, pages 639-47 (2011); Cai, Y. et al. J Clin Invest Vol. 124, pages 5129-44 (2014)) (Patel, et al., Hum Mol Genet Vol. 17, 1578-90 (2008)). Two out of the 41 total pups in this cohort were excluded, one WT and one Pkd1^{fl/fl};Pkhd1-Cre.

### Results

### Expression of PC1-CTT Suppresses Cystic Phenotype in an Orthologous Murine Model of ADPKD

The N-terminal PC1 cleavage occurs at the G protein-coupled receptor Proteolytic Site, giving rise to a 3048-aa N-terminal fragment (NTF) that remains non-covalently attached to the remaining 1254-aa C-terminal fragment (CTF), which includes PC1's 11-transmembrane domains and its cytoplasmic tail (Qian, et al., Proc Natl Acad Sci USA Vol. 99, 16981-6 (2002)). PC1 C-terminal cleavage generates several shorter PC1-CTF and C-terminal tail fragments (PC1-CTT). PC1-CTT fragments ranging from 17 to 34 kDa translocate to the nucleus and to mitochondria (Chauvet, et al., J Clin Invest Vol. 114, 1433-43(2004), Lin, et al., Sci Rep Vol. 8, 2743 (2018), Low, et al., Dev Cell Vol. 10, 57-69 (2006)). Of note, in vitro expression of a PC1-CTT construct corresponding to the final 200 aa of PC1 decreases cellular proliferation and the cross-sectional area of cysts formed by Pkd1-KO cells in 3D culture (Merrick, et al., Dev Cell Vol. 22, 197-210 (2012)). In the current study, the identical PC1-CTT sequence was expressed in Pkd1-KO DPKD mouse models and characterized the resultant phenotypes.

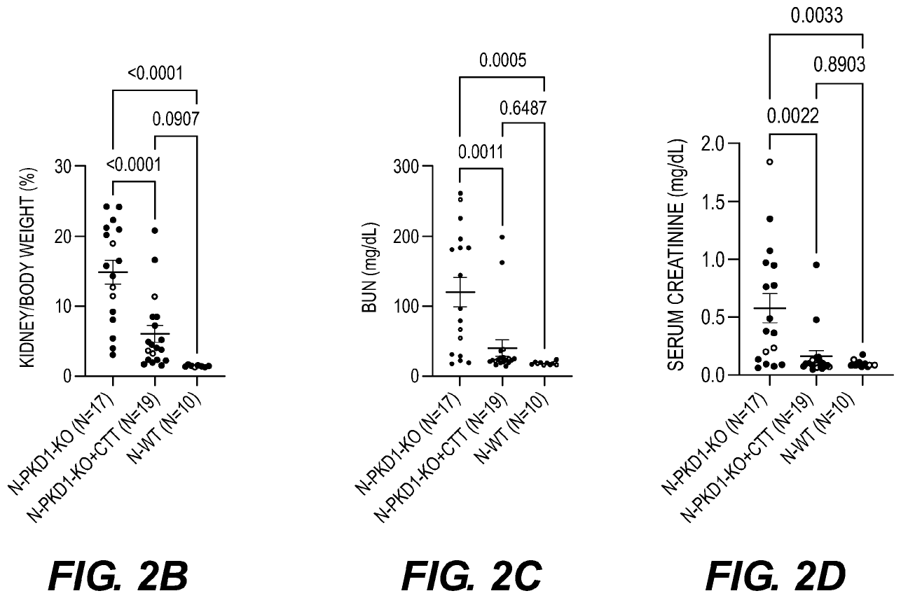

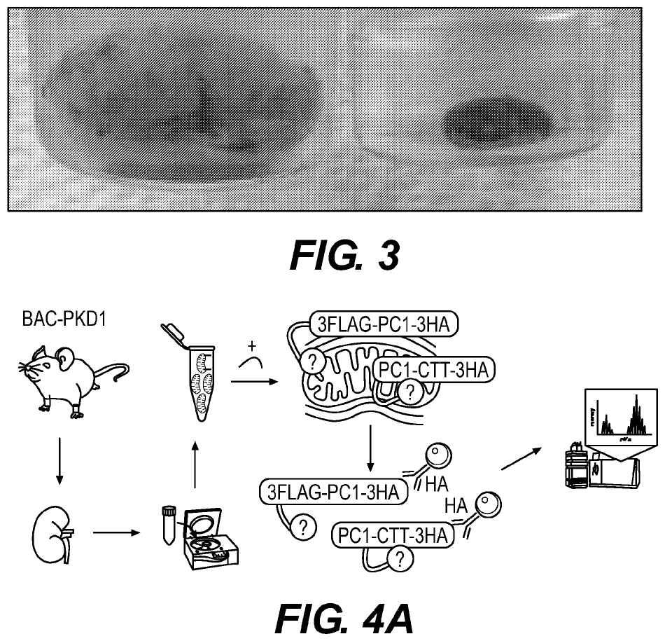

A BAC construct was generated in which the sequence that encodes the human PC1-CTT (aa 4102-4302) with an N-terminal 2XHA epitope tag (Merrick, et al., Dev Cell Vol. 22, 197-210 (2012), Merrick, et al., Hum Mol Genet Vol. 28, 16-30 (2019)) is preceded by a Flox-Stop sequence (CTT) and is inserted into the Rosa26 locus. A transgenic mouse line with this BAC transgene stably incorporated into its germline was crossed with a previously characterized conditional Pkd1-KO mouse model of ADPKD (Pkd1^{fl/fl};Pax8^{rtTA};TetO-Cre) (Ma, M., et al. Nature Genetics Vol. 45, pages 1004-12 (2013). Doxycycline induction from p28-p42 of these second-generation mice (2HA-PC1-CTT;Pkd1^{fl/fl};Pax8^{rtTA};TetO-Cre) on the C57BL/6N (“N”) background leads to CTT expression in renal epithelial cells that have undergone Cre-driven disruption of Pkd1 and therefore lack PC1 expression (FIG. 2A). Cohorts of littermates with comparable sex distributions that did or did not carry the CTT BAC transgene on the “N” background (N-Pkd1-KO+CTT vs N-Pkd1-KO mice) were generated and evaluated at 16 weeks of age (FIGS. 2B-2D). Verification of CTT expression or lack thereof, was accomplished via H&E-staining of kidney sections from the four indicated genotypes, separated by sex. Oral doxycycline was administered from weeks 4-6 and all mice were sacrificed at 16 weeks.

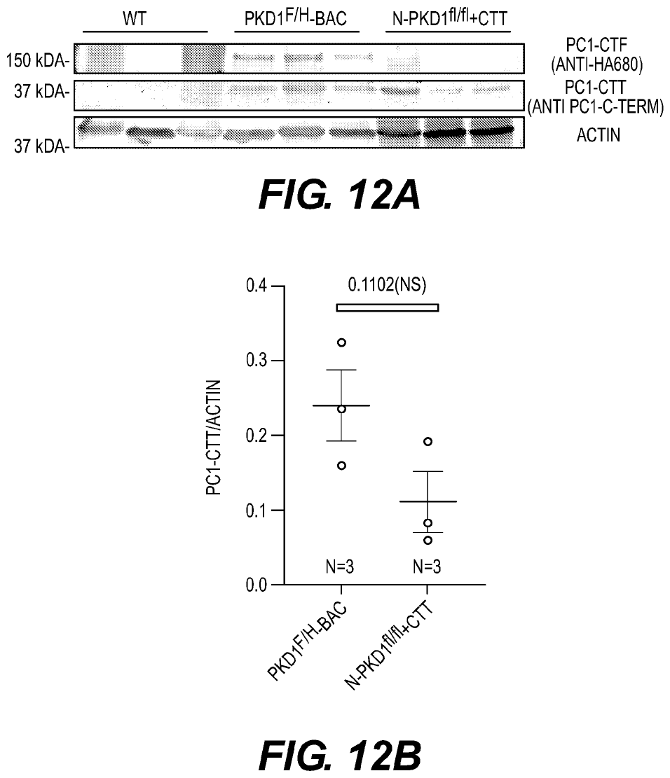

The mice that expressed CTT presented significant reduction of their cystic burden, revealed by a 60% decrease in kidney-to-body weight ratio (KW/BW) compared to N-Pkd1-KO animals that did not inherit the transgene (FIG. 2B). Consistent with the preservation of their renal size and morphology, CTT-expressing animals exhibited significant reductions in blood urea nitrogen (BUN) and serum creatinine (3 and 3.5-fold, respectively), in comparison to the N-Pkd1-KO littermates. Notably, the levels of both kidney function markers did not significantly differ between N-Pkd1-KO+CTT animals and healthy WT controls (FIGS. 2C and 2D). Quantitative analysis of CTT expression in N-Pkd1-KO+CTT mice revealed levels approximately 1.5-fold above those projected for WT animals. CTT expression levels in N-Pkd1-KO+CTT mice are comparable to those detected in Pkd1^{F/H}-BAC mice (FIG. 12A and FIG. 12B). Full-length WT PC1 is a low-abundance protein that is not reliably detectable in mouse kidneys (Lanktree, et al., Lancet 393, 919-935 (2019), Cornec-Le Gall, et al., Lancet 393, 919-935 (2019)), and its endogenous cleavage products are even less abundant and harder to detect. In this context, offspring of a founder of the Pkd1^{F/H}-BAC line (Tg248) that carries 3 copies of the BAC-Pkd1 transgene and expresses a PC1 protein with a 3× Flag tag at its N terminus and a 3×HA tag at its C terminus (Cornec-Le Gall, et al., Lancet 393, 919-935 (2019), Qian, et al., Proc Natl Acad Sci USA 99, 16981-6 (2002)) was used to quantitatively analyze CTT expression. Quantitative western blotting previously determined that Tg248 mice exhibit a 3-fold increase in tagged PC1 expression relative to offspring of a single-copy founder (Tg14) (Cornec-Le Gall, et al., Lancet 393, 919-935 (2019)). Since expression of tagged PC1 in the Tg248 line is driven by the endogenous Pkd1 promoter, it is likely that the 3 copies of the Pkd1^{F/H}-BAC transgene drive expression of the tagged protein that is roughly comparable to 1.5× the quantity of the native PC1 generated from the 2 native copies of Pkd1 encoded in the mouse genome (FIGS. 12A and 12B). Lysates from Pkd1^{F/H}-BAC mice showed the same 37-kDa C-terminal HA-tagged tail fragment band as the CTT-expressing Pkd1-KO mice (FIG. 12A), which were detected at similar levels (FIG. 12B), suggesting an upper threshold for CTT expression in the N-Pkd1-KO+CTT mice of approximately 1.5-fold above the levels expected for WT mice. This finding indicates that the suppression of cystic phenotype observed in N-Pkd1-KO+CTT mice was not a consequence of massive overexpression of CTT.

2HA-PC1-CTT (CTT) Colocalizes and Interacts with the Mitochondrial Enzyme NNT

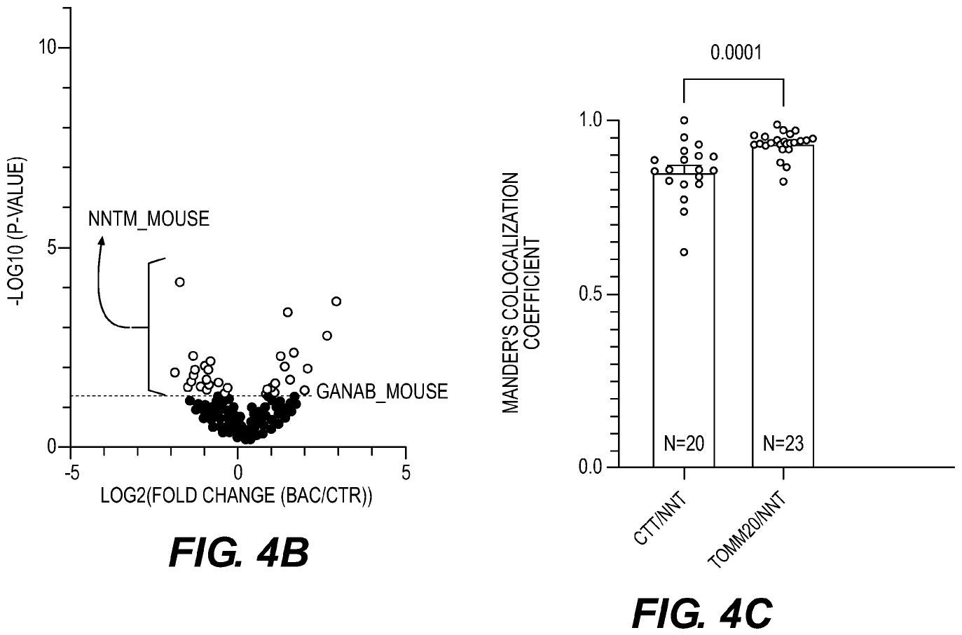

The Pkd1^{F/H}-BAC mouse model (Fedeles, S. V. et al. Nature Genetics Vol. 43, pages 639-47 (2011); Cai, Y. et al. J Clin Invest Vol. 124, pages 5129-44 (2014)); (BAC-Pkd1), generated on a mixed strain background (Fedeles, S. V. et al. Nature Genetics Vol. 43, pages 639-47 (2011)) was employed to identify pathways linking PC1-CTT expression and amelioration of disease markers. This mouse line expresses PC1 tagged with a 3×FLAG epitope at its N-terminus and with a 3×HA sequence inserted prior to the stop codon (3FLAG-PC1-3HA) at its C-terminus, under the control of its native promoter. Previous data showed that full-length PC1 localizes to mitochondria-associated endoplasmic reticulum membranes (MAMs) (Padovano, et al., Mol Biol Cell Vol. 28, 261-269 (2017)), while PC1-CTT localizes to mitochondrial matrix (Lin, et al., Sci Rep Vol. 8, 2743 (2018)). Interaction partners of these mitochondria-associated pools of PC1 and its fragments were identified. A crude mitochondrial fraction from Pkd1^{F/H}-BAC mouse kidneys, believed to contain MAM-associated full-length PC1 and PC1-CTT cleavage products, was isolated by differential centrifugation (Wieckowski, et al., Nat Protoc Vol. 4, 1582-90 (2009)). Following addition of the cleavable crosslinker DTSSP, mitochondria were solubilized, and the lysate was subjected to immunoprecipitation using anti-HA magnetic beads (FIG. 4A). Immunoblot analysis of this precipitate revealed not only the presence of the 150-kDa CTF derived from full-length 3FLAG-PC1-3HA, but also PC1-CTT-3HA fragments of 17-37 kDa. Mass spectrometric proteomic analyses of Pkd1^{F/H}-BAC and WT immunoprecipitates were compared. Potential PC1 and PC1-CTT interactors identified in this way were cross-referenced against the MitoCarta 2.0 mitochondrial proteome (Calvo, et al., Nucleic Acids Res Vol. 44, D1251-7 (2016)). The most significantly enriched interactor identified was Nicotinamide Nucleotide Transhydrogenase (NNT) (FIG. 4B, a protein that spans the mitochondrial inner membrane (IMM) and connects the proton gradient to the exchange of reducing equivalents between NADH/NAD⁺ and NADP⁺/NADPH. The location and validity of the putative CTT/NNT interaction was assessed. WT HEK293 cells were transfected with the 2HA-PC1-CTT construct and found that it colocalized extensively with endogenous NNT at the mitochondria (FIG. 4C). Interestingly, the widely used C57BL/6J (“J”) mouse strain carries a deletion of exons 7-11 in the Nnt gene, a mutation that completely abrogates NNT expression (Toye, et al., Diabetologia Vol. 48, 675-86 (2005)). Immunoblotting of total kidney lysate from WT “N” and “J” mice, confirmed the presence and absence of NNT, respectively. Immunoblotting of anti-HA immunoprecipitates from mouse kidney lysates, revealing immunoprecipitation of CTT in both “N” and “J” Pkd1-KO+CTT mice. NNT coimmunoprecipitation was detected exclusively in immunoprecipitates from N-Pkd1-KO+CTT mice.

The alleles required to produce the Pkd1-KO+/−CTT mice were moved to this NNT-deficient background (J-Pkd1-KO and J-Pkd1-KO+CTT). Immunohistochemistry confirmed absence of NNT in “J” cystic mice and showed expression of NNT in distal nephron segments and, to a lesser degree, in proximal tubules in both N-Pkd1-KO and N-Pkd1-KO+CTT mice. IHC image at 10× of N-Pkd1-KO+CTT demonstrates the preferential localization of NNT to distal convoluted tubules (DCT) and medullary tubules as compared to proximal tubule (PT). No NNT was detected in glomeruli or Bowman's capsule. This pattern reproduced the one reported in the Human Protein Atlas (Uhlen, et al., Science Vol. 347, 1260419 (2015)). To confirm the CTT/NNT interaction in vivo, anti-HA pulldowns from N-Pkd1-KO+CTT, J-Pkd1-KO+CTT and N-Pkd1-KO total kidney lysates were performed. NNT was only detected in immunoprecipitates from “N” cystic mice that express CTT and not in those derived from N-Pkd1-KO mice that express NNT but not CTT. Anti-HA immunoprecipitates from Pkd1-KO+CTT kidneys on the “J” background did not contain a 114-kDa NNT band. Taken together, these data demonstrate that PC1-CTT can localize to mitochondria and that it interacts with the IMM protein NNT in mouse kidney epithelial cells in vivo.

### The Interaction Between NNT and CTT is Important to the CTT Mediated Suppression of Cystogenesis and Tubular Proliferation

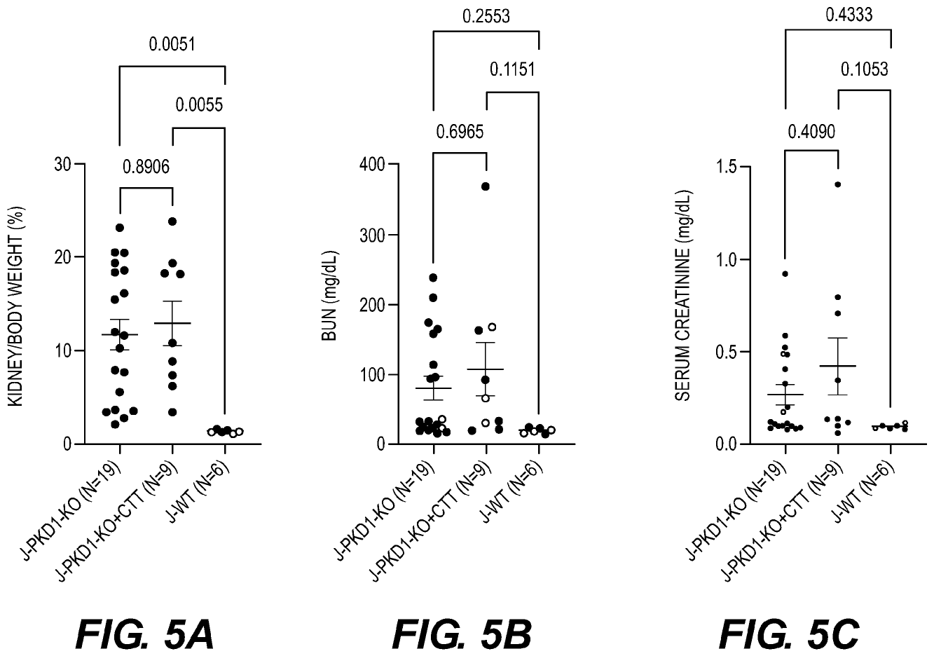

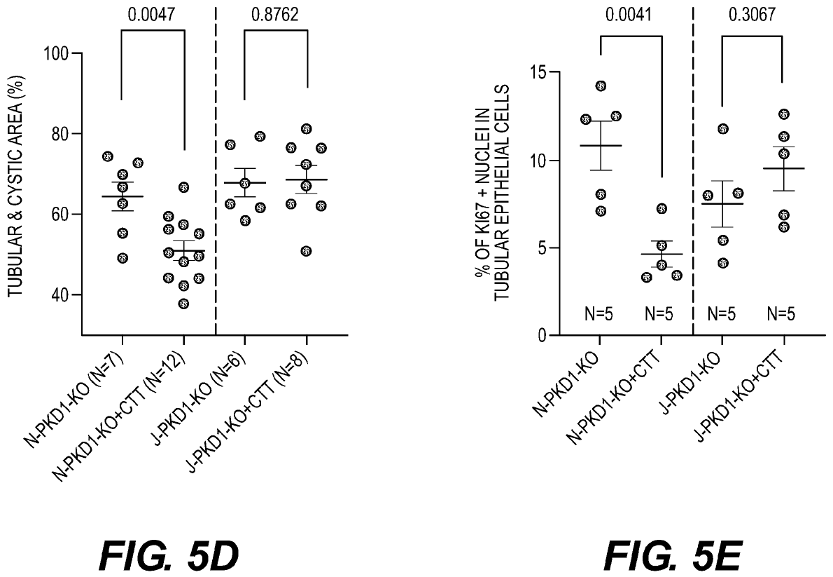

The J-Pkd1-KO+CTT mice were used to evaluate the relevance of the PC1-CTT/NNT interaction to disease progression. No significant changes in KW/BW ratio, BUN, or serum creatinine levels were observed between J-Pkd1-KO+CTT mice and their J-Pkd1-KO littermates (FIGS. 5A-5D). These models were further characterized in both backgrounds by measuring tubular and cystic area relative to the whole kidney area. This parameter was significantly smaller in Pkd1-KO+CTT vs Pkd1-KO mice exclusively on the “N” background (FIG. 5D). Additionally, immunofluorescence microscopy was performed on renal tissue from 16-week-old mice and quantified the extent of tubular epithelial cell proliferation by determining the fraction of Ki67-positive nuclei. Cyst-lining cell proliferation constitutes a hallmark of cyst expansion in ADPKD (Ma, M., et al. Nature Genetics Vol. 45, pages 1004-12 (2013), (Dong, et al., Nat Genet (2021)). While the fraction of Ki67-positive nuclei was reduced by a factor of 2.3 in Pkd1-KO+CTT compared to Pkd1-KO mice on the “N” background, proliferation levels remained equivalently elevated in these models on the “J” background (FIG. 5E). To ensure that no other factors contribute to the different responses of the “N” and “J” strains to CTT expression, assessed was whether there were any systematic differences in the level of Cre-expression or Cre-induced Pkd1-rearrangement. It was found that Pax8^{rtTA} and TetO-Cre copy numbers were randomly distributed across the four groups and did not correlate with disease severity, as defined by KW/BW ratio (FIGS. 13A-13D). FIGS. 13A-13D are bar graphs showing that Pkd1-KO+CTT and Pkd1-KO on the “N” (FIGS. 13A and 13B) and “J” (FIGS. 13C and 13D) backgrounds demonstrate random distribution of homozygosity or heterozygosity status for both Pax8′^{TA} and TetO-Cre alleles. These parameters do not correlate with phenotype severity in any of the four groups, as determined by the KW/BW ratio.

Similarly, Cre recombination efficiency, as revealed by levels of non-rearranged Pkd1 product, was the same across all four mouse groups (FIGS. 13E and 13F). FIG. 13E is a schematic representation of qPCR primers capable of exclusively detecting genomic DNA sequence encoding full-length endogenous PC1 from cells that did not undergo Cre-recombination in Pkd1-KO mice. The reverse primer is specific for Pkd1 exon (Padovano, et al., Cell Signal 72, 109634 (2020)) and the forward primer is specific to its preceding intron. Primer positions were based on the mouse genome assembly GRCm39. In the Pkd1^{fl/fl};Pax8^{rtTA};TetO-Cre model. Oral doxycycline induces activation of TetO-Cre under the control of the Pax8^{rtTA} promoter, leading to excision of the floxed exon 2-4 region and consequent inactivation of Pkd1 (Chauvet, et al., J Clin Invest 114, 1433-43 (2004), Lin, et al., Sci Rep 8, 2743 (2018)). (FIG. 13E). FIG. 13F is a bar graph of the comparative analysis of PC1 rearrangement levels across all mouse cohorts. Levels of non-rearranged WT Pkd1 was determined by extracting genomic DNA from kidney tissue from each mouse contained in the cohort followed by quantitative genomic PCR using primers described in FIG. 13E. The levels of non-rearranged WT Pkd1 were normalized to levels detected in WT controls. The fractional extent of rearrangement is unchanged across the four groups. These results demonstrate that Pax8^{rtTA} and TetO-Cre copy number do not correlate with disease severity, and PC1 rearrangement levels secondary to Cre-mediated recombination are similar across all cystic mouse cohorts

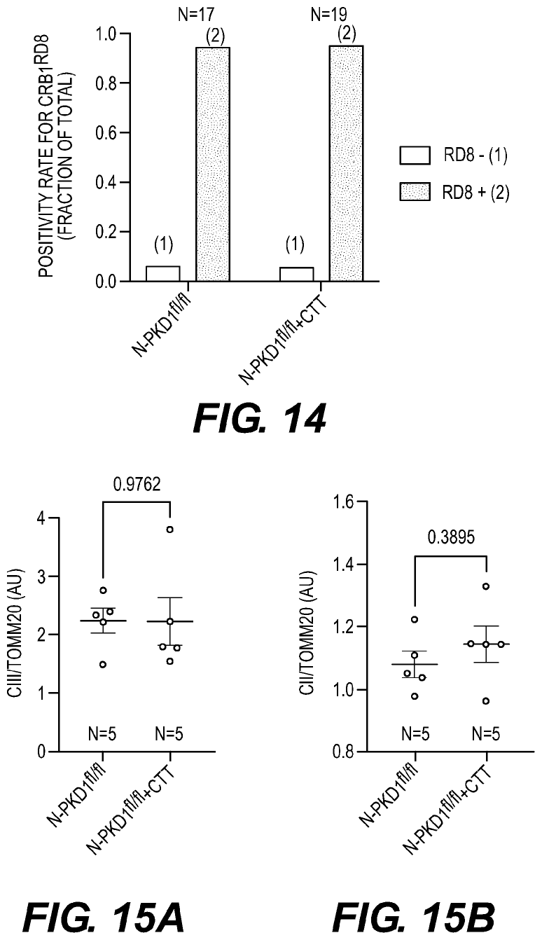

Furthermore, the Nnt mutation appears to be the major allelic difference between C57BL/6J and C57BL/6N strains (Simon, et al., Genome Biol Vol. 14, R82 (2013)) and is the only candidate genetic variation that has been directly associated with the metabolic (Toye, et al., Diabetologia Vol. 48, 675-86 (2005), Ronchi, et al., Free Radic Biol Med Vol. 63, 446-56 (2013), Fergusson, et al., Mol Metab Vol. 3, 848-54 (2014)), cardiologic (Murphy, Cell Metab Vol. 22, 363-5 (2015), Nickel, et al., Cell Metab Vol. 22, 472-84 (2015)) and renal (Usami, et al., Urolithiasis Vol. 46, 515-522 (2018)) differences observed between them. It is important to note, however, that the rd8 retinal degeneration mutant of Crb1 is detected exclusively in the “N” mice and results in a recessive ocular phenotype. This is potentially the only other phenotypically significant mutation that differs between “N” and “J” mice (Simon, et al., Genome Biol Vol. 14, R82 (2013), Mattapallil, et al., Invest Ophthalmol Vis Sci Vol. 53, 2921-7 (2012)). It was found that equal distribution of the rd8 mutant allele was present in both N-Pkd1-KO+CTT and N-Pkd1-KO mice (FIG. 14), thus excluding the possibility that skewed distributions of this mutant allele could account for the observed phenotypic difference between both groups. The frequency of the rd8 mutant allele, associated with the C57BL/6N background, was similar in N-Pkd1-KO+CTT and N-Pkd1-KO mice, as determined by standard genotyping (Consortium, Cell 78, 725 (1994)) and did not correlate with disease severity. Additionally, this observation suggests that any other potential independently assorted N versus J allelic variants are unlikely to be responsible for the observed phenotype differences.

### Expression of Polycystin-1 C-Terminal Tail (CTT) Partially Suppresses Cystic Disease in a Rapidly Progressive NNT-Expressing Orthologous Mouse Model of ADPKD

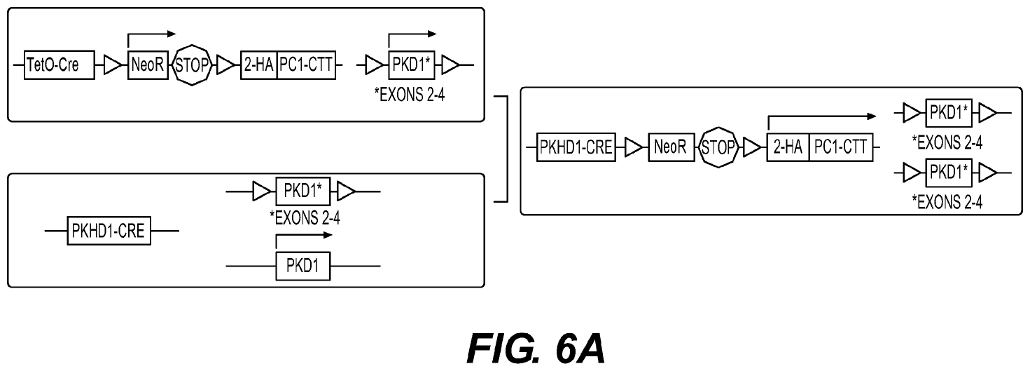

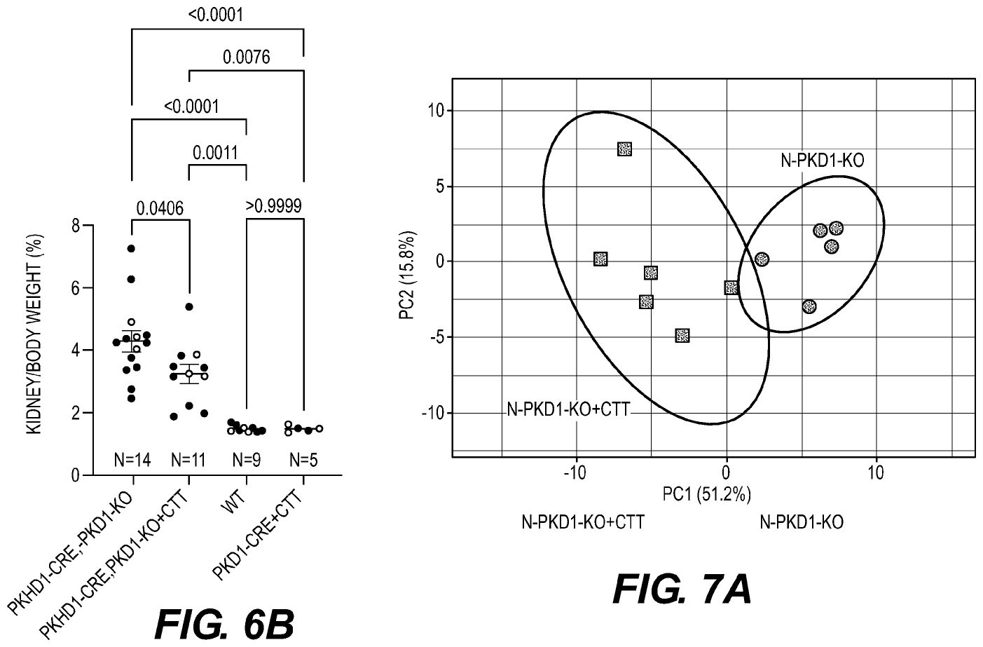

To assess the efficacy of NNT-dependent CTT suppression in an early and rapidly progressing model of cystic disease, mice in which Cre expression is driven by the kidney collecting duct-specific Pkhd1 promoter (Harris, P. C. & Torres, V. E., J Clin Invest Vol. 124, pages 2315-24 (2014)), which becomes active during embryogenesis were used. The Pkd1fl/fl;Pkhd1-Cre (Pkhd1-Cre;Pkd1-KO) (Ma, M., et al. Nature Genetics Vol. 45, pages 1004-12 (2013); Fedeles, S. V. et al. Nature Genetics Vol. 43, pages 639-47 (2011); Cai, Y. et al. J Clin Invest Vol. 124, pages 5129-44 (2014)), (Patel, et al., Hum Mol Genet Vol. 17, 1578-90 (2008)) ADPKD mouse model, generated on a C57BL/6J background, were crossed with N-Pkd1-KO+CTT mice. The resultant F₁ progeny were heterozygous for WT Nnt and included Pkhd1-Cre;Pkd1-KO+/−CTT mice (FIG. 6A). H&E-stained kidney sections from the four genotypes was conducted and all mice were sacrificed at p14. It was found that expression of the CTT partially suppressed cystic disease, revealed through reduced KW/BW ratio at p14, in this very aggressive model of renal cystic disease (FIG. 6B).

### CTT Expression Produces a Change in Metabolic Profile in the Presence of NNT

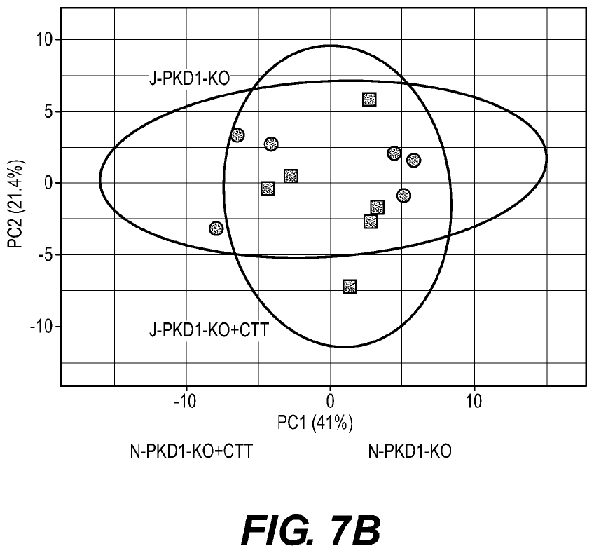

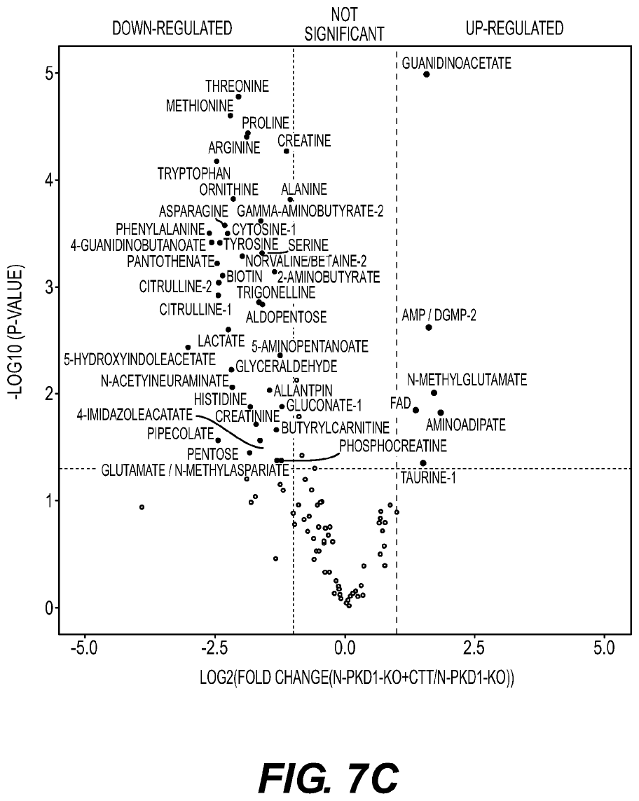

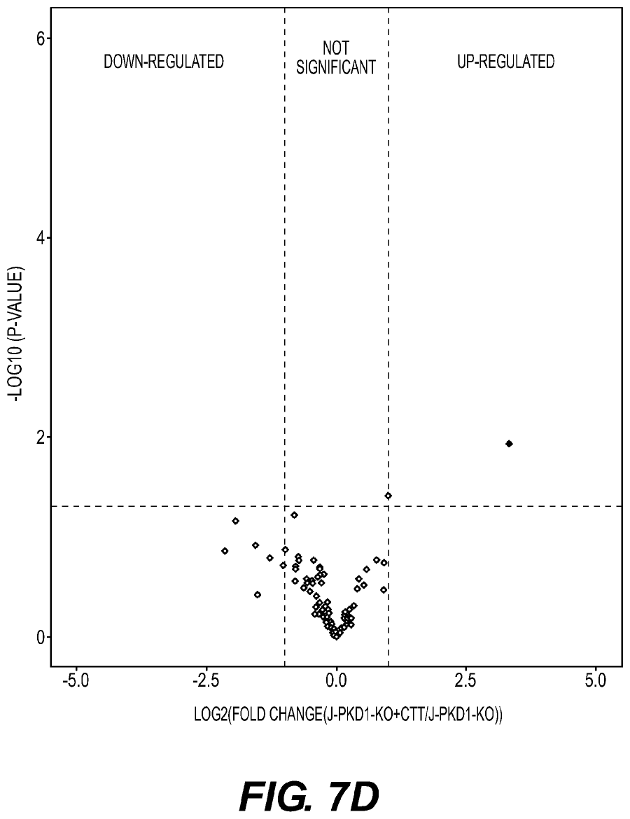

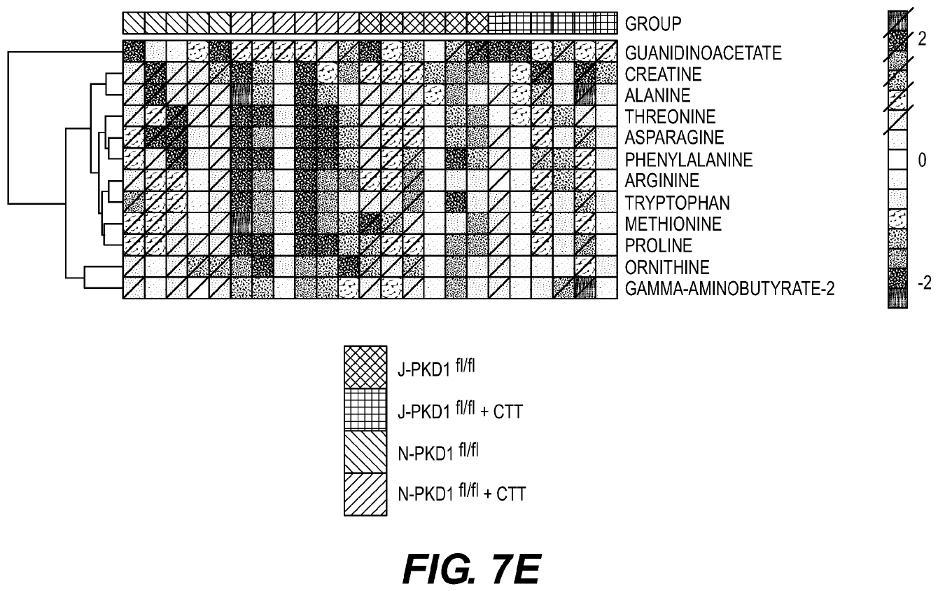

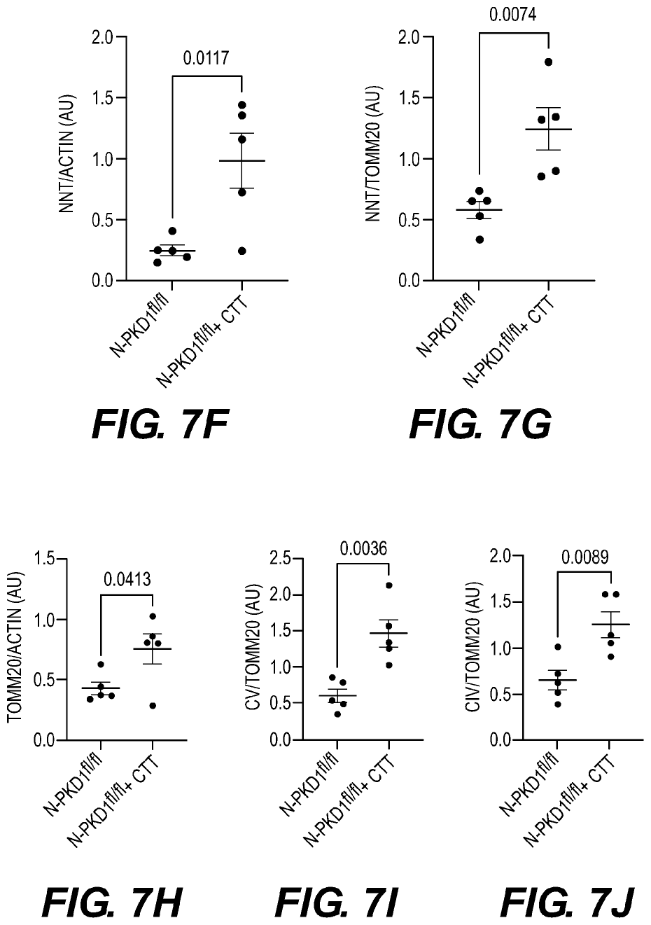

Considering the localization of 2HA-PC1-CTT to mitochondria and its NNT-dependent suppression of cystic disease, potential metabolic consequences of CTT expression in Pkd1-KO mice was evaluated by performing LC-MS-based metabolite profiling on whole kidney tissue extracts across all 4 experimental groups in the adult model at 16 weeks. While Principal Component Analysis (PCA) and hierarchical clustering revealed a distinct separation between Pkd1-KO and Pkd1-KO+CTT mice on the “N” background, it failed to distinguish among these two groups on the “J” background (FIGS. 7A and 7B). Unpaired t-test resulted in the detection of 44 metabolites that significantly changed between Pkd1-KO+CTT and Pkd1-KO mice on the “N” background (FIGS. 7C and 7D).

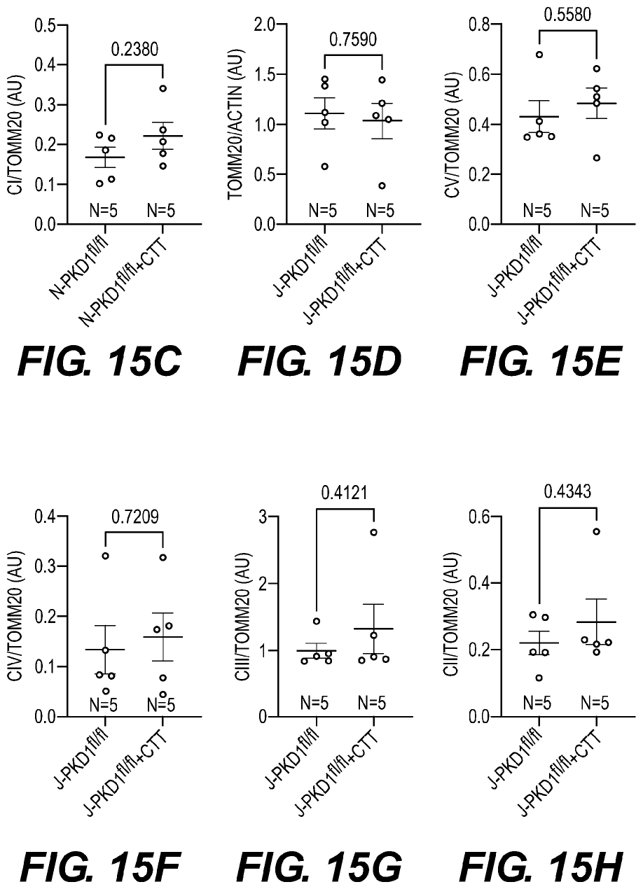

Many of the metabolites whose levels are reduced in N-Pkd1-KO+CTT mice are implicated in ADPKD pathogenesis and some of them are related to potential therapeutic targets (FIGS. 7C and 7D) such as methionine (Ramalingam, et al., Cell Metab Vol. 33, 1234-1247 e7 (2021)), lactate (Rowe, et al., Nat Med Vol. 19, 488-93 (2013), Chiaravalli, et al., J Am Soc Nephrol Vol. 27, 1958-69 (2016)), asparagine (Podrini, et al., Commun Biol Vol. 1, 194 (2018), Baliga, et al., Sci Rep Vol. 11, 6629 (2021)), and glutamate (Podrini, et al., Commun Biol Vol. 1, 194 (2018)). Observed were reductions in the levels of metabolites of the urea cycle, previously characterized as one of the most affected pathways in a pediatric ADPKD population (Baliga, et al., Sci Rep Vol. 11, 6629 (2021)). The metabolic signature associated with CTT expression in N-Pkd1-KO mice is, therefore, marked by the reversal of dysregulated metabolites that are associated with ADPKD. It was determined whether CTT and CTT/NNT interactions alter the inventories of proteins that potentially affect mitochondrial function (FIGS. 7F-7J). Immunoblotting revealed a 4-fold increase in NNT expression (NNT/actin) in 16-week-old CTT-expressing mice compared to N-Pkd1-KO littermates. This rise resulted from both increased mitochondrial mass (TOMM20/actin) and increased NNT expression at a “per mitochondrion” level (NNT/TOMM20) (FIGS. 7F-7J). Comparable findings were observed in tissue from cystic human kidneys, which exhibited a significant decrease in NNT protein expression as compared to healthy kidney tissue. Furthermore, western blotting employing a “mitococktail” antibody that interrogates the levels of stably assembled mitochondrial membrane complexes demonstrated increased levels of assembled ATP-synthase (complex V or CV) and cytochrome c oxidase (complex IV or CIV) at a “per mitochondrion” level, as revealed by an increase in both CV/TOMM20 and CIV/TOMM20 ratios in N-Pkd1-KO+CTT expressing mice (FIGS. 7F-7J). Additional comparisons of mitochondrial components in cystic mice that do or do not express CTT were conducted. No difference in mitochondrial mass or in mitochondrial complex assembly was detected as a consequence of CTT expression on the “J” background (FIGS. 15A-15H) suggesting that the effects observed in the “N” background involve the CTT/NNT interaction. FIGS. 15A-15H are dot plots showing comparisons of normalized band intensities representative of both mitochondrial complex assembly and mitochondrial mass that did not differ between cystic mice that do or do not express CTT in both “N” (FIGS. 15A-15C) and “J” (FIGS. 15D-15H) backgrounds.

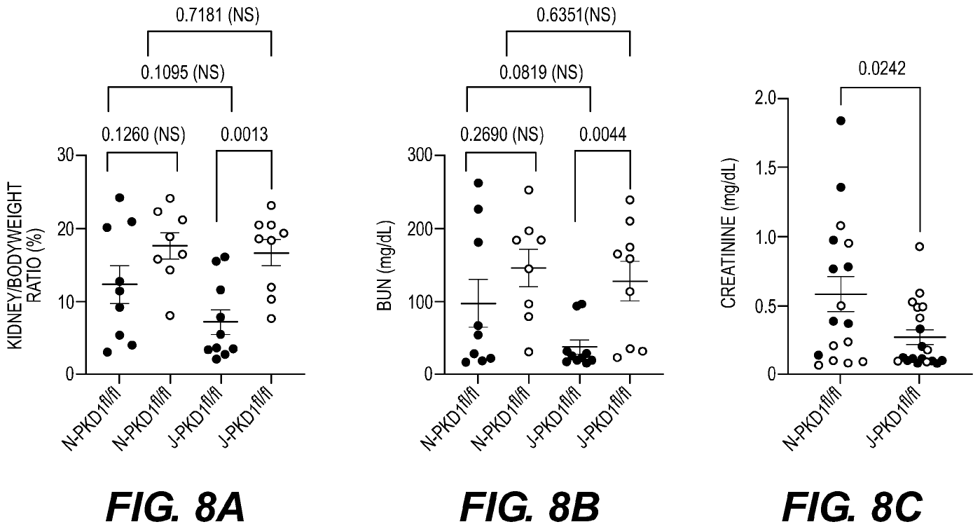

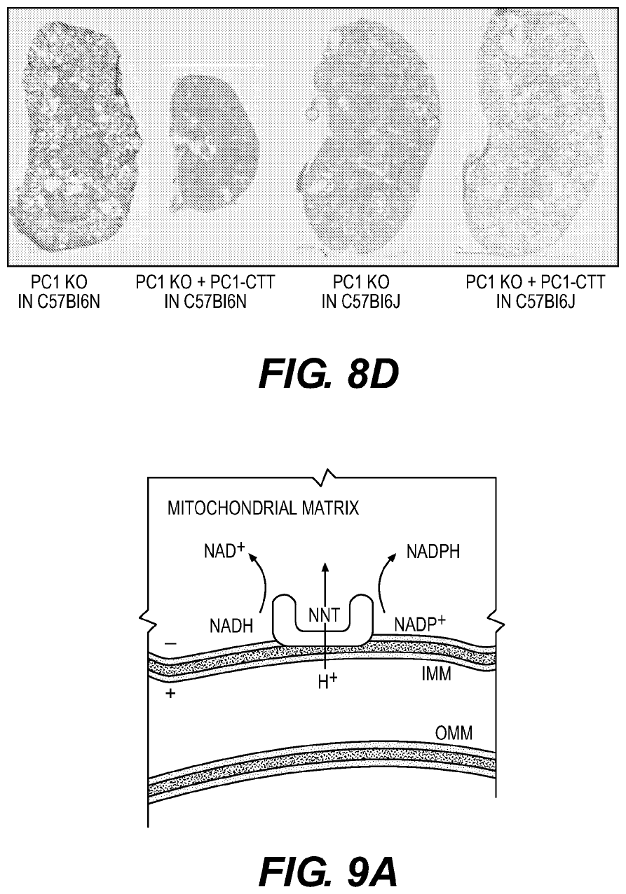

Additionally, the change of background from J to N, independent of PC1-CTT re-expression, can lead to important differences in terms of cyst progression and gender dimorphism: PKD1 KO in the J background presents a significantly increased severity in males compared to females. In the N background, this difference is less pronounced and not significant when comparing males and females. In the N background, animals were frequently observed to exhibit a dilated pelvis that was also not a common finding in the animals of the J background. Further, an overall more aggressive phenotype was observed in the N background, that is revealed by roughly a 2-fold increase in serum creatinine. See FIGS. 8A-8D.

### CTT Expression Modulates Mitochondrial Redox and Increases NNT Enzymatic Activity

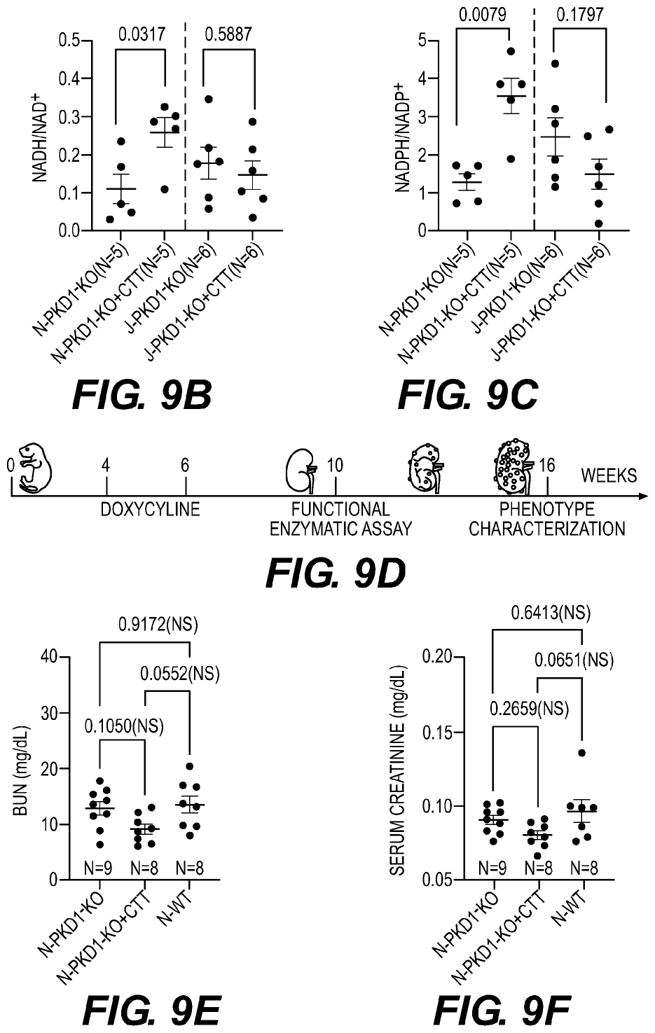

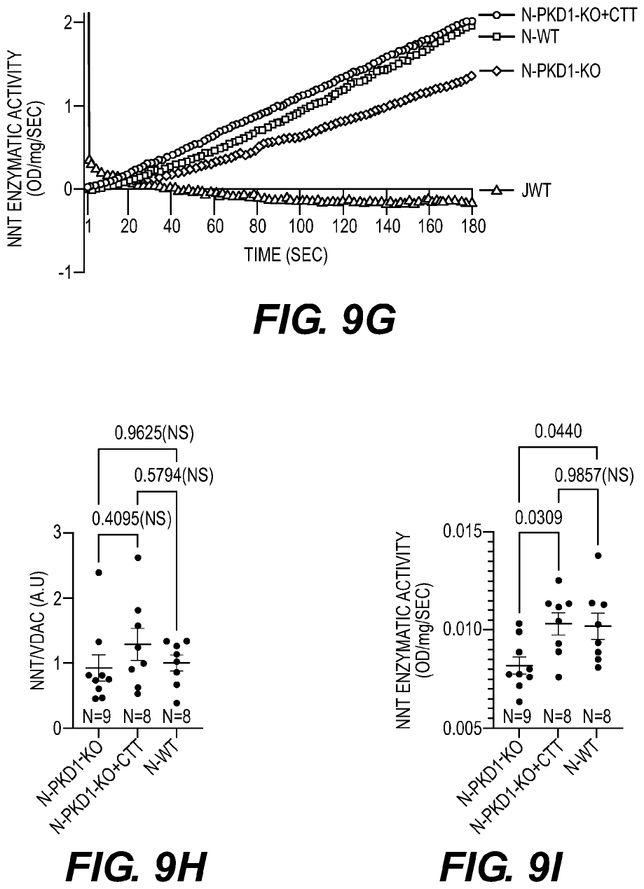

To assess whether and how the CTT might alter NNT activity, targeted LC-MS was performed to quantify NAD(P)(H) levels in kidney homogenates from 16-week-old Pkd1-KO+CTT and Pkd1-KO mice on both backgrounds (FIG. 9A). N-Pkd1-KO+CTT exhibited an increase in NADPH/NADP+ and NADH/NAD+ ratios when compared to N-Pkd1-KO mice (FIGS. 9B and 9C), while CTT expression in J-Pkd1-KO mice did not affect either ratio. While NAD(P)(H) measurements provide indirect insights into the level of NNT function, these values alone do not directly report NNT enzymatic activity since many processes contribute to determining NAD(P)(H) levels. The NNT activity was directly assessed. To ensure that the assessment of NNT enzymatic activity was not influenced by the cystic phenotype, the experiments were conducted in pre-cystic, 10-week-old mice (Ma, M., et al. Nature Genetics Vol. 45, pages 1004-12 (2013) (FIGS. 9D-9F).

Immunoblotting of a mitochondrial fraction from fresh kidney tissue revealed no significant differences in NNT expression among 10-week-old N-Pkd1-KO+CTT, N-Pkd1-KO and N-WT mice (FIG. 9H), in contrast to differences observed in 16-week-old animals (FIGS. 7F-7J). The assessment of NNT enzymatic activity was performed using a standard kinetic spectrophotometric assay that detects NNT-mediated reduction of the NAD analog APAD (Shimomura, et al., Methods Enzymol Vol. 457, 451-80 (2009)). A 20% decrease was detected in NNT enzymatic activity in N-Pkd1-KO mice compared to “N” WT controls (FIGS. 9G and 9I). Furthermore, CTT expression in N-Pkd1-KO mice rescued NNT enzymatic activity to the same level observed in the healthy “N” controls (FIGS. 9G and 9I). Of note, this assay was performed on mitochondria extracted from whole-kidney tissue, which includes multiple cell types in addition to Cre-expressing tubular cells. Hence, the magnitude of the observed difference is likely an underestimation of the true effect manifested in Cre-expressing cells.

The PC1-CTT sequence contains amino acids 4104-4303. The amino acids contained in the MTS. Original identified as a nuclear localization sequence (NLS) in Chauvet, V et al., The Journal of clinical investigation vol. 114,10 (2004): 1433-43. doi:10.1172/JCI21753 having the sequence: LRRLRLWMGLSKVKEFRHKVR (SEQ ID NO:98), a longer sequence with the added 5 amino acids: MVELFLRRLRLWMGLSKVKEFRHKVR (SEQ ID NO:99) was identified as a mitochondrial targeting sequence in Lin, C. et al. Scientific reports vol. 8,1 2743. 9 Feb. 2018, doi:10.1038/s41598-018-20856-6.

To test the importance of the mitochondrial localization signal to the biological activity of PC1-CTT, cell culture experiments were conducted using a construct lacking the mitochondrial targeting sequence (MTS; or mitochondrial localization sequence, MLS) defined by amino acids 4134-4154:

(SEQ ID NO: 100)
VILRWRYHALRGELYRPAWEPQDYEMVELFFEGMEPLPSRSSRGSKVSPDV
PPPSAGSDASHPSTSSSQLDGLSVSLGRLGTRCEPEPSRLQAVFEALLTQF
DRLNQATEDVYQLEQQLHSLQGRRSSRAPAGSSRGPSPGLRPALPSRLARA
SRGVDLATGPSRTPLRAKNKVHPSST
also referred to as PC1-CTTΔMTS).

FIGS. 11A and 11B are graphs illustrating generation of a TERT immortalized Pkd−/− cell line and its use to compare the activity of PC1-CTT, EV, PC1-CTTΔMTS, and EV(-NADPH). Results show that the NNT activity in this cell line, which lacks polycystin-1 expression, can be substantially increased by expressing the polycystin-1 C-terminal tail (PC1-CTT), and this effect does not occur if the polycystin-1 C-terminal tail lacks the mitochondrial localization sequence (PC1-CTTAMTS). A kinetic spectrophotometric assay previously developed for ex vivo studies, was optimized and validated, for in vitro systems. This assay was performed on fresh mitochondrial lysate and detected the time-dependent, NNT-driven transfer of a reducing equivalent from NADPH to the synthetic substrate APAD. This permitted assessment of NNT activity of transiently transfected immortalized Pkd1KO/KO mouse cells. These cells were created by transducing primary cultures of tubule epithelial cells isolated from Pkd1KO/KO mice with a construct encoding TERT.

Immunoblotting of mitochondrial extracts confirmed that PC1-CTT accumulates within the mitochondria (FIGS. 11A and 11B). Transient transfection of PC1-CTT construct in Pkd1KO/KO mouse cells revealed a significant increase in NNT enzymatic activity as compared to that of an empty expression vector (EV, pcDNA3.1+ expression vector) or transfection control. Transfection with the PC1-CTT construct lacking aa 4134-4154 (ΔMTS), as described above, did not produce the same effect and NNT activity levels were similar to that obtained from control cells transfected with the empty expression vector (EV).

In conclusion, it was shown that expressing the C-terminal 200-aa segment (CTT) of PC1 in mature mice that serve as an orthologous model of ADPKD is sufficient to suppress the development of the cystic phenotype and to preserve renal function and morphology, as evidenced by BUN and serum creatinine levels that were comparable to levels detected in healthy controls. Mass spectrometric analyses identified NNT as the most significant CTT binding partner and this interaction was further confirmed by coimmunoprecipitation. A previous analysis showed that PC1-CTT localizes specifically to the mitochondrial matrix or matrix-facing surface of the inner mitochondrial membrane (IMM) (Lin, et al., Sci Rep Vol. 8, 2743 (2018)), consistent with the predicted topological requirements of this new interaction. It was shown that the suppression of the cystic phenotype produced by CTT expression is dependent upon the availability of this interaction. Similarly, unbiased metabolomics revealed differentially clustering metabolites between Pkd1-KO+CTT and Pkd1-KO mice only on the “N” background. Notably, CTT expression in the presence of NNT leads to decreased tubular cell proliferation, increased mitochondrial mass, altered redox modulation, and increased assembly of CIV and ATP synthase at a “per mitochondrion” level. Previous studies have shown that increased quantities of stably assembled electron transport chain (ETC) complexes correlate with increased ETC activity and increased levels of oxidative phosphorylation (Sieber, et al., Cell Vol. 164, 420-32 (2016), Sing, et al., Cell Vol. 158, 1293-1308 (2014)). In concert with the 4-fold reduction in lactate levels in CTT-expressing N-Pkd1-KO mice, these data support the interpretation that the CTT rescue model exhibits a profound shift towards oxidative phosphorylation as the predominant source of ATP generation.

The finding that NNT expression alone does not protect against cyst formation, but that CTT expression suppresses cyst formation in an NNT-dependent manner suggests that NNT acts as a disease modifier rather than as a primary participant in cystogenic pathways. Changes were observed in redox metabolites and a significant increase in NNT enzymatic activity in the CTT-expressing model compared to N-Pkd1-KO mice. To make substrates accessible to NNT in the kinetic assay, mitochondrial membrane integrity is disrupted, which eliminates the NNT-driving proton motive force. Thus, it is possible that CTT modulates the directional kinetics of the enzymatic reaction. While the CTT-dependent increase in NNT activity in its forward-mode could lead to increased NADPH levels and enhanced anti-oxidative defense, it is hypothesized that CTT favors increased “reverse-mode” NNT activity, a finding that has been reported in pathological conditions (Murphy, Cell Metab Vol. 22, 363-5 (2015), Nickel, et al., Cell Metab Vol. 22, 472-84 (2015)). and in NADPH-rich environments (Kampjut & Sazanov, Nature Vol. 573, 291-295 (2019)). “Reverse-mode” NNT activity leads to increased oxidative phosphorylation by increasing both NADH levels and the magnitude of the proton gradient. It also leads to a decrease in NADPH levels and thus impairs antioxidant defense (Murphy, Cell Metab Vol. 22, 363-5 (2015)). This effect could suppress cystogenesis by increasing the susceptibility of oxidatively stressed cyst epithelial cells to apoptosis-inducing oxidative damage (Maser, et al., J Am Soc Nephrol Vol. 13, 991-999 (2002)).

The above-described non-limiting examples are described in Laura Onuchic, V. P. et al. bioRxiv 2021.12.21.473680; doi: https://doi.org/10.1101/2021.12.21.473680, the entire contents of which, including all supplemental materials, are specifically incorporated by reference in its entireties.

Unless defined otherwise, all technical and scientific terms used herein have the same meanings as commonly understood by one of skill in the art to which the disclosed invention belongs. Publications cited herein and the materials for which they are cited are specifically incorporated by reference in their entireties.

Those skilled in the art will recognize, or be able to ascertain using no more than routine experimentation, many equivalents to the specific embodiments of the invention described herein. Such equivalents are intended to be encompassed by the following claims.

We claim:

1. A polypeptide comprising SEQ ID NO:1 or a functional fragment or variant thereof and a heterologous sequence, optionally packaged in or otherwise associated with a delivery vehicle.

2. The polypeptide of claim 1 comprising the delivery vehicle.

3. A polypeptide comprising SEQ ID NO:1 or a functional fragment or variant thereof packaged in or otherwise associated with a delivery vehicle, optionally wherein the polypeptide comprises a heterologous sequence.

4. The polypeptide of claim 3 comprising the heterologous sequence.

5. A variant polypeptide comprising at least 70% and less than 100% sequence identity to SEQ ID NO:1 or functional fragment thereof, optionally wherein the polypeptide comprises a heterologous sequence, is packaged in or otherwise associated with a delivery vehicle, or a combination thereof.

6. The polypeptide of claim 5, comprising the heterologous sequence.

7. The polypeptide of claims 5 or 6, comprising the delivery vehicle.

8. The polypeptide of any one of claims 1-7, wherein the heterologous sequence comprises one or more of a protein transduction domain, fusogenic polypeptide, targeting signal, expression and/or purification tag.

9. The polypeptide of any one of claims 1-8, wherein the variant comprises at least 75% sequence identity of SEQ ID NO:1, or a functional fragment thereof.

10. The polypeptide of any one of claims 1-9, wherein the variant or fragment is between 25 and 200 amino acids inclusive, or any subrange or specific integer therebetween.

11. The polypeptide of any one of claims 1-10, wherein the polypeptide can interact with nicotinamide nucleotide transhydrogenase (NNT), optionally wherein interaction comprises the ability to co-immunoprecipitate.

12. The polypeptide of any one of claims 1-8, comprising a mutated PEST motif with reduce activity.

13. The polypeptide of any one of claims 1-12 comprising a mitochondrial localization signal.

14. The polypeptide of claim 13, wherein the mitochondrial localization signal comprises the amino acid sequence of SEQ ID NOS:98 or 99, or a variant thereof with a least 70% sequence identity thereto.

15. The polypeptide of any one of claims 1-14 comprising a heterologous mitochondrial localization signal.

16. The polypeptide of claim 15, wherein the amino acid sequence of SEQ ID NO:98 and/or SEQ ID NO:99 is absent.

17. The polypeptide of claim 16, comprising the amino acid sequence of SEQ ID NO:100 or a fragment or variant thereof with at least 70% sequence identity thereto.

18. The polypeptide of claim 16 comprising a variant of the amino acid sequence of SEQ ID NO:1 wherein the amino acid sequence of SEQ ID NOS:98 or 99 is deleted, and the heterologous mitochondrial localization signal is inserted in its place or appended to the N- or C-terminus of the polypeptide.

19. A nucleic acid comprising a nucleic acid encoding the polypeptide of any one of claims 1-18, optionally packaged in a delivery vehicle.

20. The nucleic acid of claim 19 comprising or encoding a TOP or TOP-like motif.

21. A nucleic acid comprising a nucleic acid encoding a therapeutic polypeptide operably linked to a TOP or TOP-like motif or its encoding sequence, optionally packaged in a delivery vehicle.

22. The nucleic acid of claim 21, wherein the therapeutic polypeptide comprises SEQ ID NO:1 or a functional fragment or variant thereof.

23. The nucleic acid of any one of claims 19-22, wherein the TOP or TOP-like motif comprises at least 4 pyrimidines beginning within four nucleotides of the transcriptional start site, optionally beginning at the transcription start site.

24. The nucleic acid of claims 22 or 23, wherein the TOP or TOP-like motif comprises the nucleic acid sequence of the underlined portion of any of SEQ ID NOS:21-52 of Table 1, and/or any of SEQ ID NOS:21-52 or 87.

25. The nucleic acid of any one of claims 19-24, wherein the nucleic acid is RNA or DNA.

26. The nucleic acid of any one of claims 19-25, wherein the nucleic acid comprises an expression control sequence(s).

27. The nucleic acid of any one of claims 19-26, wherein the nucleic acid is a vector.

28. The nucleic acid of claim 27, wherein the nucleic acid is a viral vector.

29. The nucleic acid of any one of claims 19-28, wherein the nucleic acid is mRNA.

30. The nucleic acid of any one of claims 19-29, wherein the nucleic acid comprises a promotor.

31. The nucleic acid of claim 30, wherein the promotor is a kidney-specific promoter.

32. The nucleic acid of any one of claims 19-31 comprising one or more of a protein transduction domain, fusogenic polypeptide, or targeting signal conjugated thereto.

33. The nucleic acid of any one of claims 19-32 comprising the delivery vehicle.

34. The polypeptide of any one of claims 1-18 or nucleic acid of any one of claims 19-33, wherein the delivery vehicle is formed of polymeric particles, inorganic particles, silica particles, liposomes, micelles, or multilamellar vesicles, optionally wherein the delivery vehicles comprise one or more of a protein transduction domain, fusogenic polypeptide, or targeting signal conjugated thereto.

35. A pharmaceutical composition comprising the any one of claims 1-18 or nucleic acid of any one of claims 19-34 alone or packaged in a delivery vehicle optionally formed from formed of polymeric particles, inorganic particles, silica particles, liposomes, micelles, or multilamellar vesicles, optionally wherein the delivery vehicles comprise one or more of a protein transduction domain, fusogenic polypeptide, or targeting signal conjugated thereto.

36. A method of treating a subject in need thereof comprising administering the subject an effective amount of the pharmaceutical composition of claim 35.

37. The method of claim 36, wherein the subject has a genetic disorder.

38. The method of claim 37, wherein the genetic disorder is Autosomal Dominant Polycystic Kidney Disease (ADPKD).

39. The method of claim 38, wherein the composition is administered by a retrograde ureteral approach.

40. A method of treating Autosomal Dominant Polycystic Kidney Disease (ADPKD) comprising administering a subject with ADPKD the pharmaceutical composition of claim 35.

41. A method of treating a subject with a disease characterized by increase mTOR activity comprising administering the subject a pharmaceutical composition comprising the nucleic acid of any one of claims 21-34.

42. The method of claim 41, wherein the disease is selected from ADPKD, arthritis, insulin resistance, osteoporosis, cancer, and mTOR-opathies optionally selected from tuberous sclerosis complex (TSC), focal cortical dysplasia type II (FCDII), hemimegaloencephaly (HME), polyhydramnios, megalocephaly, and symptomatic epilepsy (PMSE) syndrome.

43. The method of claim 41, wherein the disease is a genetic disorder, and the therapeutic polypeptide is a wildtype copy or other fragment or variant thereof that restores the function or bioactivity lost by the mutated gene/protein of the genetic disorder.

44. The method of claim 41, wherein the disease is a cancer.

45. The method of claim 44, wherein the therapeutic polypeptide is cytotoxic to cancer cells, an antimicrobial peptide, a peptide that targets a transduction pathway, a peptide that targets the cell cycle, a peptide that induces cell death, a peptide that targets a transcription factor, and/or a peptide that counters an aspect of mTORC1 activation.

46. The method of claims 44 or 45, wherein the therapeutic polypeptide is selected from the peptides of Table 2.


SEQUENCE LISTING (100 sequences)

SEQ ID NO: 1 - protein, 200 aa, synthetic construct
VILRWRYHALRGELYRPAWEPQDYEMVELFLRRLRLWMGLSKVKEFRHKVRFEGMEPLPSRSSRGSKVSPDVPPPSAGSDASHPSTSSSQLDGLSVSLGRLGTRCEPEPSRLQAVFEALLTQFDRLNQATEDVYQLEQQLHSLQGRRSSRAPAGSSRGPSPGLRPALPSRLARASRGVDLATGPSRTPLRAKNKVHPSST

SEQ ID NO: 2 - protein, 4303 aa, Homo sapiens
MPPAAPARLALALGLGLWLGALAGGPGRGCGPCEPPCLCGPAPGAACRVNCSGRGLRTLGPALRIPADATALDVSHNLLRALDVGLLANLSALAELDISNNKISTLEEGIFANLFNLSEINLSGNPFECDCGLAWLPRWAEEQQVRVVQPEAATCAGPGSLAGQPLLGIPLLDSGCGEEYVACLPDNSSGTVAAVSFSAAHEGLLQPEACSAFCFSTGQGLAALSEQGWCLCGAAQPSSASFACLSLCSGPPPPPAPTCRGPTLLQHVFPASPGATLVGPHGPLASGQLAAFHIAAPLPVTATRWDFGDGSAEVDAAGPAASHRYVLPGRYHVTAVLALGAGSALLGTDVQVEAAPAALELVCPSSVQSDESLDLSIQNRGGSGLEAAYSIVALGEEPARAVHPLCPSDTEIFPGNGHCYRLVVEKAAWLQAQEQCQAWAGAALAMVDSPAVQRFLVSRVTRSLDVWIGFSTVQGVEVGPAPQGEAFSLESCQNWLPGEPHPATAEHCVRLGPTGWCNTDLCSAPHSYVCELQPGGPVQDAENLLVGAPSGDLQGPLTPLAQQDGLSAPHEPVEVMVFPGLRLSREAFLTTAEFGTQELRRPAQLRLQVYRLLSTAGTPENGSEPESRSPDNRTQLAPACMPGGRWCPGANICLPLDASCHPQACANGCTSGPGLPGAPYALWREFLFSVPAGPPAQYSVTLHGQDVLMLPGDLVGLQHDAGPGALLHCSPAPGHPGPRAPYLSANASSWLPHLPAQLEGTWACPACALRLLAATEQLTVLLGLRPNPGLRLPGRYEVRAEVGNGVSRHNLSCSFDVVSPVAGLRVIYPAPRDGRLYVPTNGSALVLQVDSGANATATARWPGGSVSARFENVCPALVATFVPGCPWETNDTLFSVVALPWLSEGEHVVDVVVENSASRANLSLRVTAEEPICGLRATPSPEARVLQGVLVRYSPVVEAGSDMVFRWTINDKQSLTFQNVVFNVIYQSAAVFKLSLTASNHVSNVTVNYNVTVERMNRMQGLQVSTVPAVLSPNATLALTAGVLVDSAVEVAFLWTFGDGEQALHQFQPPYNESFPVPDPSVAQVLVEHNVMHTYAAPGEYLLTVLASNAFENLTQQVPVSVRASLPSVAVGVSDGVLVAGRPVTFYPHPLPSPGGVLYTWDFGDGSPVLTQSQPAANHTYASRGTYHVRLEVNNTVSGAAAQADVRVFEELRGLSVDMSLAVEQGAPVVVSAAVQTGDNITWTFDMGDGTVLSGPEATVEHVYLRAQNCTVTVGAASPAGHLARSLHVLVFVLEVLRVEPAACIPTQPDARLTAYVTGNPAHYLFDWTFGDGSSNTTVRGCPTVTHNFTRSGTFPLALVLSSRVNRAHYFTSICVEPEVGNVTLQPERQFVQLGDEAWLVACAWPPFPYRYTWDFGTEEAAPTRARGPEVTFIYRDPGSYLVTVTASNNISAANDSALVEVQEPVLVTSIKVNGSLGLELQQPYLFSAVGRGRPASYLWDLGDGGWLEGPEVTHAYNSTGDFTVRVAGWNEVSRSEAWLNVTVKRRVRGLVVNASRTVVPLNGSVSFSTSLEAGSDVRYSWVLCDRCTPIPGGPTISYTFRSVGTFNIIVTAENEVGSAQDSIFVYVLQLIEGLQVVGGGRYFPTNHTVQLQAVVRDGTNVSYSWTAWRDRGPALAGSGKGFSLTVLEAGTYHVQLRATNMLGSAWADCTMDFVEPVGWLMVAASPNPAAVNTSVTLSAELAGGSGVVYTWSLEEGLSWETSEPFTTHSFPTPGLHLVTMTAGNPLGSANATVEVDVQVPVSGLSIRASEPGGSFVAAGSSVPFWGQLATGTNVSWCWAVPGGSSKRGPHVTMVFPDAGTFSIRLNASNAVSWVSATYNLTAEEPIVGLVLWASSKVVAPGQLVHFQILLAAGSAVTFRLQVGGANPEVLPGPRFSHSFPRVGDHVVSVRGKNHVSWAQAQVRIVVLEAVSGLQVPNCCEPGIATGTERNFTARVQRGSRVAYAWYFSLQKVQGDSLVILSGRDVTYTPVAAGLLEIQVRAFNALGSENRTLVLEVQDAVQYVALQSGPCFTNRSAQFEAATSPSPRRVAYHWDFGDGSPGQDTDEPRAEHSYLRPGDYRVQVNASNLVSFFVAQATVTVQVLACREPEVDVVLPLQVLMRRSQRNYLEAHVDLRDCVTYQTEYRWEVYRTASCQRPGRPARVALPGVDVSRPRLVLPRLALPVGHYCFVFVVSFGDTPLTQSIQANVTVAPERLVPIIEGGSYRVWSDTRDLVLDGSESYDPNLEDGDQTPLSFHWACVASTQREAGGCALNFGPRGSSTVTIPRERLAAGVEYTFSLTVWKAGRKEEATNQTVLIRSGRVPIVSLECVSCKAQAVYEVSRSSYVYLEGRCLNCSSGSKRGRWAARTFSNKTLVLDETTTSTGSAGMRLVLRRGVLRDGEGYTFTLTVLGRSGEEEGCASIRLSPNRPPLGGSCRLFPLGAVHALTTKVHFECTGWHDAEDAGAPLVYALLLRRCRQGHCEEFCVYKGSLSSYGAVLPPGFRPHFEVGLAVVVQDQLGAAVVALNRSLAITLPEPNGSATGLTVWLHGLTASVLPGLLRQADPQHVIEYSLALVTVLNEYERALDVAAEPKHERQHRAQIRKNITETLVSLRVHTVDDIQQIAAALAQCMGPSRELVCRSCLKQTLHKLEAMMLILQAETTAGTVTPTAIGDSILNITGDLIHLASSDVRAPQPSELGAESPSRMVASQAYNLTSALMRILMRSRVLNEEPLTLAGEEIVAQGKRSDPRSLLCYGGAPGPGCHFSIPEAFSGALANLSDVVQLIFLVDSNPFPFGYISNYTVSTKVASMAFQTQAGAQIPIERLASERAITVKVPNNSDWAARGHRSSANSANSVVVQPQASVGAVVTLDSSNPAAGLHLQLNYTLLDGHYLSEEPEPYLAVYLHSEPRPNEHNCSASRRIRPESLQGADHRPYTFFISPGSRDPAGSYHLNLSSHFRWSALQVSVGLYTSLCQYFSEEDMVWRTEGLLPLEETSPRQAVCLTRHLTAFGASLFVPPSHVRFVFPEPTADVNYIVMLTCAVCLVTYMVMAAILHKLDQLDASRGRAIPFCGQRGRFKYEILVKTGWGRGSGTTAHVGIMLYGVDSRSGHRHLDGDRAFHRNSLDIFRIATPHSLGSVWKIRVWHDNKGLSPAWFLQHVIVRDLQTARSAFFLVNDWLSVETEANGGLVEKEVLAASDAALLRFRRLLVAELQRGFFDKHIWLSIWDRPPRSRFTRIQRATCCVLLICLFLGANAVWYGAVGDSAYSTGHVSRLSPLSVDTVAVGLVSSVVVYPVYLAILFLFRMSRSKVAGSPSPTPAGQQVLDIDSCLDSSVLDSSFLTFSGLHAEQAFVGQMKSDLFLDDSKSLVCWPSGEGTLSWPDLLSDPSIVGSNLRQLARGQAGHGLGPEEDGFSLASPYSPAKSFSASDEDLIQQVLAEGVSSPAPTQDTHMETDLLSSLSSTPGEKTETLALQRLGELGPPSPGLNWEQPQAARLSRTGLVEGLRKRLLPAWCASLAHGLSLLLVAVAVAVSGWVGASFPPGVSVAWLLSSSASFLASFLGWEPLKVLLEALYFSLVAKRLHPDEDDTLVESPAVTPVSARVPRVRPPHGFALFLAKEEARKVKRLHGMLRSLLVYMLFLLVTLLASYGDASCHGHAYRLQSAIKQELHSRAFLAITRSEELWPWMAHVLLPYVHGNQSSPELGPPRLRQVRLQEALYPDPPGPRVHTCSAAGGFSTSDYDVGWESPHNGSGTWAYSAPDLLGAWSWGSCAVYDSGGYVQELGLSLEESRDRLRFLQLHNWLDNRSRAVFLELTRYSPAVGLHAAVTLRLEFPAAGRALAALSVRPFALRRLSAGLSLPLLTSVCLLLFAVHFAVAEARTWHREGRWRVLRLGAWARWLLVALTAATALVRLAQLGAADRQWTRFVRGRPRRFTSFDQVAQLSSAARGLAASLLFLLLVKAAQQLRFVRQWSVFGKTLCRALPELLGVTLGLVVLGVAYAQLAILLVSSCVDSLWSVAQALLVLCPGTGLSTLCPAESWHLSPLLCVGLWALRLWGALRLGAVILRWRYHALRGELYRPAWEPQDYEMVELFLRRLRLWMGLSKVKEFRHKVRFEGMEPLPSRSSRGSKVSPDVPPPSAGSDASHPSTSSSQLDGLSVSLGRLGTRCEPEPSRLQAVFEALLTQFDRLNQATEDVYQLEQQLHSLQGRRSSRAPAGSSRGPSPGLRPALPSRLARASRGVDLATGPSRTPLRAKNKVHPSST

SEQ ID NO: 3 - protein, 11 aa, synthetic construct
GWHDAEDAGAP

SEQ ID NO: 4 - protein, 11 aa, synthetic construct
YGRKKRRQRRR

SEQ ID NO: 5 - protein, 9 aa, synthetic construct
RKKRRQRRR

SEQ ID NO: 6 - protein, 7 aa, synthetic construct
RRRRRRR

SEQ ID NO: 7 - protein, 16 aa, synthetic construct
RQIKIWFQNRRMKWKK

SEQ ID NO: 8 - protein, 19 aa, synthetic construct
HHHHRKKRRQRRRRHHHHH

SEQ ID NO: 9 - protein, 30 aa, synthetic construct
MVKSKIGSWILVLFVAMWSDVGLCKKRPKP

SEQ ID NO: 10 - protein, 27 aa, synthetic construct
GALFLGFLGAAGSTMGAWSQPKKKRKV

SEQ ID NO: 11 - protein, 8 aa, synthetic construct
DYKDDDDK

SEQ ID NO: 12 - protein, 6 aa, synthetic construct
YPYDVP

SEQ ID NO: 13 - protein, 10 aa, synthetic construct
ILKKATAYIL

SEQ ID NO: 14 - protein, 10 aa, synthetic construct
EQKLISEEDL

SEQ ID NO: 15 - protein, 7 aa, synthetic construct
GQSSRSS

SEQ ID NO: 16 - protein, 6 aa, synthetic construct
GSGSGS

SEQ ID NO: 17 - protein, 6 aa, synthetic construct
ASGGGS

SEQ ID NO: 18 - protein, 10 aa, synthetic construct
GGGGSGGGGS

SEQ ID NO: 19 - protein, 20 aa, synthetic construct
GGGGSGGGGSGGGGSGGGGS

SEQ ID NO: 20 - protein, 15 aa, synthetic construct
GGGGSGGGGSGGGGS

SEQ ID NO: 21 - RNA, 20 nt, synthetic construct
  source 1..20: mol_type = other RNA
ctctttccgttcctagcgca

SEQ ID NO: 22 - RNA, 20 nt, synthetic construct
  source 1..20: mol_type = other RNA
ctcttccgtcgcagagtttc

SEQ ID NO: 23 - RNA, 20 nt, synthetic construct
  source 1..20: mol_type = other RNA
cctctttccttgcctaacgc

SEQ ID NO: 24 - RNA, 20 nt, synthetic construct
  source 1..20: mol_type = other RNA
cctcttttccgtggcgcctc

SEQ ID NO: 25 - RNA, 20 nt, synthetic construct
  source 1..20: mol_type = other RNA
ctctttccagccagcgccga

SEQ ID NO: 26 - RNA, 20 nt, synthetic construct
  source 1..20: mol_type = other RNA
cctttctccggcggccggga

SEQ ID NO: 27 - RNA, 20 nt, synthetic construct
  source 1..20: mol_type = other RNA
ctttttttcaggcggccggg

SEQ ID NO: 28 - RNA, 20 nt, synthetic construct
  source 1..20: mol_type = other RNA
ctctttccggtgtggagtct

SEQ ID NO: 29 - RNA, 20 nt, synthetic construct
  source 1..20: mol_type = other RNA
cttttcccagcagccgccaa

SEQ ID NO: 30 - RNA, 20 nt, synthetic construct
  source 1..20: mol_type = other RNA
ccttttccggtcgcggcgct

SEQ ID NO: 31 - RNA, 20 nt, synthetic construct
  source 1..20: mol_type = other RNA
cctcttttaccaaggacccg

SEQ ID NO: 32 - RNA, 20 nt, synthetic construct
  source 1..20: mol_type = other RNA
cttttcctccttggctgtct

SEQ ID NO: 33 - RNA, 20 nt, synthetic construct
  source 1..20: mol_type = other RNA
cttctctcgccaggcgtcct

SEQ ID NO: 34 - RNA, 20 nt, synthetic construct
  source 1..20: mol_type = other RNA
ctttcgccgccggacgccgc

SEQ ID NO: 35 - RNA, 20 nt, synthetic construct
  source 1..20: mol_type = other RNA
ctctcttcttttccggctgg

SEQ ID NO: 36 - RNA, 20 nt, synthetic construct
  source 1..20: mol_type = other RNA
ctttctttctccagcagccg

SEQ ID NO: 37 - RNA, 20 nt, synthetic construct
  source 1..20: mol_type = other RNA
ctctctcctcccgccgccca

SEQ ID NO: 38 - RNA, 20 nt, synthetic construct
  source 1..20: mol_type = other RNA
ctttcccaggcggctgccga

SEQ ID NO: 39 - RNA, 20 nt, synthetic construct
  source 1..20: mol_type = other RNA
cttttccaagcggctgccga

SEQ ID NO: 40 - RNA, 20 nt, synthetic construct
  source 1..20: mol_type = other RNA
cctttctcgctccccggccg

SEQ ID NO: 41 - RNA, 20 nt, synthetic construct
  source 1..20: mol_type = other RNA
cttcttcctcggcgctgcct

SEQ ID NO: 42 - RNA, 20 nt, synthetic construct
  source 1..20: mol_type = other RNA
ctctttctgccatcttggcg

SEQ ID NO: 43 - RNA, 20 nt, synthetic construct
  source 1..20: mol_type = other RNA
ctttttcgcaacgggtttgc

SEQ ID NO: 44 - RNA, 20 nt, synthetic construct
  source 1..20: mol_type = other RNA
ctttttcctctcttcagcgt

SEQ ID NO: 45 - RNA, 20 nt, synthetic construct
  source 1..20: mol_type = other RNA
ctcttccgccgcagccgccg

SEQ ID NO: 46 - RNA, 20 nt, synthetic construct
  source 1..20: mol_type = other RNA
ccccttctccccggcggtta

SEQ ID NO: 47 - RNA, 20 nt, synthetic construct
  source 1..20: mol_type = other RNA
cttttttcgcgctacccggg

SEQ ID NO: 48 - RNA, 20 nt, synthetic construct
  source 1..20: mol_type = other RNA
ctctttcccttcggtgtggt

SEQ ID NO: 49 - RNA, 20 nt, synthetic construct
  source 1..20: mol_type = other RNA
ctttccctggtgtgattccg

SEQ ID NO: 50 - RNA, 20 nt, synthetic construct
  source 1..20: mol_type = other RNA
cttttttccgcccgctcccc

SEQ ID NO: 51 - RNA, 20 nt, synthetic construct
  source 1..20: mol_type = other RNA
cttttccgcccgctcccccc

SEQ ID NO: 52 - RNA, 20 nt, synthetic construct
  source 1..20: mol_type = other RNA
cttttccgcccgctcccccc

SEQ ID NO: 53-55 - skipped (3 entries)

SEQ ID NO: 56 - RNA, 11 nt, synthetic construct
  source 1..11: mol_type = other RNA
cctctttcctt

SEQ ID NO: 57 - RNA, 10 nt, synthetic construct
  source 1..10: mol_type = other RNA
cctcttttcc

SEQ ID NO: 58-64 - skipped (7 entries)

SEQ ID NO: 65 - RNA, 12 nt, synthetic construct
  source 1..12: mol_type = other RNA
cttttcctcctt

SEQ ID NO: 66-67 - skipped (2 entries)

SEQ ID NO: 68 - RNA, 14 nt, synthetic construct
  source 1..14: mol_type = other RNA
ctctcttcttttcc

SEQ ID NO: 69 - RNA, 12 nt, synthetic construct
  source 1..12: mol_type = other RNA
ctttctttctcc

SEQ ID NO: 70 - RNA, 12 nt, synthetic construct
  source 1..12: mol_type = other RNA
ctctctcctccc

SEQ ID NO: 71-73 - skipped (3 entries)

SEQ ID NO: 74 - RNA, 10 nt, synthetic construct
  source 1..10: mol_type = other RNA
cttcttcctc

SEQ ID NO: 75-76 - skipped (2 entries)

SEQ ID NO: 77 - RNA, 15 nt, synthetic construct
  source 1..15: mol_type = other RNA
ctttttcctctcttc

SEQ ID NO: 78 - skipped

SEQ ID NO: 79 - RNA, 12 nt, synthetic construct
  source 1..12: mol_type = other RNA
ccccttctcccc

SEQ ID NO: 80 - skipped

SEQ ID NO: 81 - RNA, 12 nt, synthetic construct
  source 1..12: mol_type = other RNA
ctctttcccttc

SEQ ID NO: 82-86 - skipped (5 entries)

SEQ ID NO: 87 - RNA, 20 nt, synthetic construct
  source 1..20: mol_type = other RNA
cyyyyyycscyssscgscsn

SEQ ID NO: 88 - protein, 40 aa, synthetic construct
LPSRSSRGSKVSPDVPPPSAGSDASHPSTSSSQLDGLSVS

SEQ ID NO: 89 - protein, 10 aa, synthetic construct
DVPPPSAGSD

SEQ ID NO: 90 - RNA, 20 nt, synthetic construct
  source 1..20: mol_type = other RNA
aagtcataaacggcgctctg

SEQ ID NO: 91 - RNA, 20 nt, synthetic construct
  source 1..20: mol_type = other RNA
cagtacagggtaggctgctc

SEQ ID NO: 92 - RNA, 21 nt, synthetic construct
  source 1..21: mol_type = other RNA
tccatagaagacactgggacc

SEQ ID NO: 93 - RNA, 21 nt, synthetic construct
  source 1..21: mol_type = other RNA
agtaaagtgtacaggatcggc

SEQ ID NO: 94 - RNA, 20 nt, synthetic construct
  source 1..20: mol_type = other RNA
tggtgtgacagtgacttggg

SEQ ID NO: 95 - RNA, 20 nt, synthetic construct
  source 1..20: mol_type = other RNA
gtcctcagtgtagcccaaga

SEQ ID NO: 96 - RNA, 21 nt, synthetic construct
  source 1..21: mol_type = other RNA
tctgtcatcttgccctgttcc

SEQ ID NO: 97 - RNA, 21 nt, synthetic construct
  source 1..21: mol_type = other RNA
gttgcactcaaatgggttccc

SEQ ID NO: 98 - protein, 21 aa, synthetic construct
LRRLRLWMGLSKVKEFRHKVR

SEQ ID NO: 99 - protein, 26 aa, synthetic construct
MVELFLRRLRLWMGLSKVKEFRHKVR

SEQ ID NO: 100 - protein, 179 aa, synthetic construct
VILRWRYHALRGELYRPAWEPQDYEMVELFFEGMEPLPSRSSRGSKVSPDVPPPSAGSDASHPSTSSSQLDGLSVSLGRLGTRCEPEPSRLQAVFEALLTQFDRLNQATEDVYQLEQQLHSLQGRRSSRAPAGSSRGPSPGLRPALPSRLARASRGVDLATGPSRTPLRAKNKVHPSST